(19) United States
(12) Patent Application Publication
(10) Pub. No.: US 2018/0044415 A1
(43) Pub. Date: Feb. 15, 2018

(54) ANTI-DLL3 CHIMERIC ANTIGEN RECEPTORS AND METHODS OF USE

(71) Applicant: ABBVIE STEMCENTRX LLC, NORTH CHICAGO, IL (US)

(72) Inventors: PAUL ANTHONY ESCARPE, SAN BRUNO, CA (US); SCOTT J. DYLLA, EMERALD HILLS, CA (US); DAVID LIU, SAN FRANCISCO, CA (US); ROBERT A. STULL, ALAMEDA, CA (US)

(73) Assignee: ABBVIE STEMCENTRX LLC, NORTH CHICAGO, IL (US)

(21) Appl. No.: 15/553,102

(22) PCT Filed: Feb. 23, 2016
(86) PCT No.: PCT/US16/19192

§ 371 Date: Aug. 23, 2017

Related U.S. Application Data
(60) Provisional application No. 62/119,793, filed on Feb. 23, 2015.
(60) Provisional application No. 62/241,662, filed on Oct. 14, 2015.
(60) Provisional application No. 62/296,560, filed on Feb. 17, 2016.

(51) Int. Cl. C07K 16/28; C12N 5/0783; A61K 35/17; C12N 15/86; C07K 14/725; C07K 14/705

(52) U.S. Cl. CPC C07K 16/28; C07K 14/7051; C07K 14/70578; C07K 16/2815; A61K 35/17; C12N 15/86; C12N 5/0638; C07K 2317/622; C07K 2317/56; C07K 2319/02; C07K 2319/03; C12N 2740/15043; C07K 2317/24; C07K 2319/74; C12N 2510/00

(57) ABSTRACT

Provided herein are novel anti-DLL3 chimeric antigen receptors and methods of using the same to treat proliferative disorders.

### CROSS REFERENCED APPLICATIONS

This claims the benefit of U.S. Provisional Application No. 62/119,793 filed on 23 Feb. 2015, U.S. Provisional Application No. 62/241,662 filed on 14 Oct. 2015, and U.S. Provisional Application No. 62/296,560 filed on 17 Feb. 2016, each of which is incorporated herein by reference in its entirety.

### SEQUENCE LISTING

This application contains a sequence listing which has been submitted in ASCII format via EFS-Web and is hereby incorporated by reference in its entirety. Said ASCII copy, created on Feb. 19, 2016, is named S69697_1250WO_sc1605pct_ST25.txt and is 612 KB (626,688 bytes) in size.

### FIELD OF THE INVENTION

The present invention generally relates to adoptive immunotherapy comprising the use of novel chimeric antigen receptors incorporating a DLL3 binding domain. In preferred embodiments the disclosed chimeric antigen receptors are useful for the treatment or prophylaxis of proliferative disorders and any recurrence or metastasis thereof.

### BACKGROUND OF THE INVENTION

Differentiation and proliferation of stem cells and progenitor cells are normal ongoing processes that act in concert to support tissue growth during organogenesis, cell repair and cell replacement. The system is tightly regulated to ensure that only appropriate signals are generated based on the needs of the organism. Cell proliferation and differentiation normally occur only as necessary for the replacement of damaged or dying cells or for growth. However, disruption of these processes can be triggered by many factors including the under- or overabundance of various signaling chemicals, the presence of altered microenvironments, genetic mutations or a combination thereof. Disruption of normal cellular proliferation and/or differentiation can lead to various disorders including proliferative diseases such as cancer.

Conventional therapeutic treatments for cancer include chemotherapy, radiotherapy and immunotherapy. Often these treatments are ineffective and surgical resection may not provide a viable clinical alternative. Limitations in the current standard of care are particularly evident in those cases where patients undergo first line treatments and subsequently relapse. In such cases refractory tumors, often aggressive and incurable, frequently arise. The overall survival rates for many solid tumors have remained largely unchanged over the years due, at least in part, to the failure of existing therapies to prevent relapse, tumor recurrence and metastasis. There remains therefore a great need to develop more targeted and potent therapies for proliferative disorders. The current invention addresses this need.

### SUMMARY OF THE INVENTION

In a broad aspect the present invention provides novel chimeric antigen receptors (CARs) comprising a DLL3 binding domain that specifically binds to human DLL3 protein (DLL3 CARs). In certain embodiments the DLL3 protein is expressed on tumor initiating cells. Through genetic modification (e.g., transduction) the DLL3 CAR are expressed on cytotoxic lymphocytes (preferably autologous) to provide DLL3 sensitive lymphocytes that are used to target and kill DLL3 positive tumor cells. As will be discussed extensively herein the CARs of the instant invention generally comprise an extracellular domain comprising a DLL3 binding domain (which may be derived from an anti-DLL3 antibody), a transmembrane domain and an intracellular signaling domain that activates certain lymphocytes and generates an immune response directed to DLL3 positive tumor cells. Selected embodiments of the invention comprise immunoactive host cells expressing the disclosed CARs and various polynucleotide sequences and vectors encoding the DLL3 CARs of the invention. Yet other aspects include methods of enhancing T lymphocyte or natural killer (NK) cell activity in an individual and treating an individual suffering from cancer by introducing into the individual host cells expressing DLL3 CAR molecules. Such aspects specifically include the treatment of lung cancer (e.g., small cell lung cancer), melanoma, breast cancer, prostate cancer, colon cancer, renal cell carcinoma, ovarian cancer, neuroblastoma, rhabdomyosarcoma, leukemia and lymphoma.

As discussed in more detail below, the term “antibody” as used herein shall be held to mean intact antibodies (e.g., IgG or IgM) as well as any immunoreactive fragments (e.g., Fab fragments) or immunoreactive constructs or derivatives thereof (e.g., scFv). In certain embodiments the DLL3 binding domains (and DLL3 CARs) of the instant invention will comprise scFv constructs and, in preferred embodiments, will comprise scFv constructs that compete for binding with antibodies comprising heavy and light chain variable regions as disclosed herein. In other preferred embodiments the DLL3 binding domains (and DLL3 CARs) of the invention will comprise scFv constructs comprising heavy and light chain variable regions disclosed herein or fragments thereof. As such, for the purposes of the instant disclosure the term “antibody” shall be used generally and will expressly be held to include immunoreactive fragments, constructs or derivatives thereof unless otherwise contextually dictated.

In selected aspects of the invention the CAR binding domain binds specifically to hDLL3 and will be derived from, comprise or compete for binding with an antibody or antibody fragment comprising: a light chain variable region (VL) of SEQ ID NO: 21 and a heavy chain variable region (VH) of SEQ ID NO: 23; or a VL of SEQ ID NO: 25 and a VH of SEQ ID NO: 27; or a VL of SEQ ID NO: 29 and a VH of SEQ ID NO: 31; or a VL of SEQ ID NO: 33 and a VH of SEQ ID NO: 35; or a VL of SEQ ID NO: 37 and a VH of SEQ ID NO: 39; or a VL of SEQ ID NO: 41 and a VH of SEQ ID NO: 43; or a VL of SEQ ID NO: 45 and a VH of SEQ ID NO: 47; or a VL of SEQ ID NO: 49 and a VH of SEQ ID NO: 51; or a VL of SEQ ID NO: 53 and a VH of SEQ ID NO: 55; or a VL of SEQ ID NO: 57 and a VH of SEQ ID NO: 59; or a VL of SEQ ID NO: 61 and a VH of SEQ ID NO: 63; or a VL of SEQ ID NO: 65 and a VH of SEQ ID NO: 67; or a VL of SEQ ID NO: 69 and a VH of SEQ ID NO: 71; or a VL of SEQ ID NO: 73 and a VH of SEQ ID NO: 75; or a VL of SEQ ID NO: 77 and a VH of SEQ ID NO: 79; or a VL of SEQ ID NO: 81 and a VH of SEQ ID NO: 83; or a VL of SEQ ID NO: 85 and a VH of SEQ ID NO: 87; or a VL of SEQ ID NO: 89 and a VH of SEQ ID NO: 91; or a VL of SEQ ID NO: 93 and a VH of SEQ ID NO: 95; or a VL of SEQ ID NO: 97 and a VH of SEQ ID NO: 99; or a VL of SEQ ID NO: 101 and a VH of SEQ ID NO: 103; or a VL of SEQ ID NO: 105 and a VH of SEQ ID NO: 107; or a VL of SEQ ID NO: 109 and a VH of SEQ ID NO: 111; or a VL of SEQ ID NO: 113 and a VH of SEQ ID NO: 115; or a VL of SEQ ID NO: 117 and a VH of SEQ ID NO: 119; or a VL of SEQ ID NO: 121 and a VH of SEQ ID NO: 123; or a VL of SEQ ID NO: 125 and a VH of SEQ ID NO: 127; or a VL of SEQ ID NO: 129 and a VH of SEQ ID NO: 131; or a VL of SEQ ID NO: 133 and a VH of SEQ ID NO: 135; or a VL of SEQ ID NO: 137 and a VH of SEQ ID NO: 139; or a VL of SEQ ID NO: 141 and a VH of SEQ ID NO: 143; or a VL of SEQ ID NO: 145 and a VH of SEQ ID NO: 147; or a VL of SEQ ID NO: 149 and a VH of SEQ ID NO: 151; or a VL of SEQ ID NO: 153 and a VH of SEQ ID NO: 155; or a VL of SEQ ID NO: 157 and a VH of SEQ ID NO: 159; or a VL of SEQ ID NO: 161 and a VH of SEQ ID NO: 163; or a VL of SEQ ID NO: 165 and a VH of SEQ ID NO: 167; or a VL of SEQ ID NO: 169 and a VH of SEQ ID NO: 171; or a VL of SEQ ID NO: 173 and a VH of SEQ ID NO: 175; or a VL of SEQ ID NO: 177 and a VH of SEQ ID NO: 179; or a VL of SEQ ID NO: 181 and a VH of SEQ ID NO: 183; or a VL of SEQ ID NO: 185 and a VH of SEQ ID NO: 187; or a VL of SEQ ID NO: 189 and a VH of SEQ ID NO: 191; or a VL of SEQ ID NO: 193 and a VH of SEQ ID NO: 195; or a VL of SEQ ID NO: 197 and a VH of SEQ ID NO: 199; or a VL of SEQ ID NO: 201 and a VH of SEQ ID NO: 203; or a VL of SEQ ID NO: 205 and a VH of SEQ ID NO: 207; or a VL of SEQ ID NO: 209 and a VH of SEQ ID NO: 211; or a VL of SEQ ID NO: 213 and a VH of SEQ ID NO: 215; or a VL of SEQ ID NO: 217 and a VH of SEQ ID NO: 219; or a VL of SEQ ID NO: 221 and a VH of SEQ ID NO: 223; or a VL of SEQ ID NO: 225 and a VH of SEQ ID NO: 227; or a VL of SEQ ID NO: 229 and a VH of SEQ ID NO: 231; or a VL of SEQ ID NO: 233 and a VH of SEQ ID NO: 235; or a VL of SEQ ID NO: 237 and a VH of SEQ ID NO: 239; or a VL of SEQ ID NO: 241 and a VH of SEQ ID NO: 243; or a VL of SEQ ID NO: 245 and a VH of SEQ ID NO: 247; or a VL of SEQ ID NO: 249 and a VH of SEQ ID NO: 251; or a VL of SEQ ID NO: 253 and a VH of SEQ ID NO: 255; or a VL of SEQ ID NO: 257 and a VH of SEQ ID NO: 259; or a VL of SEQ ID NO: 261 and a VH of SEQ ID NO: 263; or a VL of SEQ ID NO: 265 and a VH of SEQ ID NO: 267; or a VL of SEQ ID NO: 269 and a VH of SEQ ID NO: 271; or a VL of SEQ ID NO: 273 and a VH of SEQ ID NO: 275; or a VL of SEQ ID NO: 277 and a VH of SEQ ID NO: 279; or a VL of SEQ ID NO: 281 and a VH of SEQ ID NO: 283; or a VL of SEQ ID NO: 285 and a VH of SEQ ID NO: 287; or a VL of SEQ ID NO: 289 and a VH of SEQ ID NO: 291; or a VL of SEQ ID NO: 293 and a VH of SEQ ID NO: 295; or a VL of SEQ ID NO: 297 and a VH of SEQ ID NO: 299; or a VL of SEQ ID NO: 301 and a VH of SEQ ID NO: 303; or a VL of SEQ ID NO: 305 and a VH of SEQ ID NO: 307; or a VL of SEQ ID NO: 309 and a VH of SEQ ID NO: 311; or a VL of SEQ ID NO: 313 and a VH of SEQ ID NO: 315; or a VL of SEQ ID NO: 317 and a VH of SEQ ID NO: 319; or a VL of SEQ ID NO: 321 and a VH of SEQ ID NO: 323; or a VL of SEQ ID NO: 325 and a VH of SEQ ID NO: 327; or a VL of SEQ ID NO: 329 and a VH of SEQ ID NO: 331; or a VL of SEQ ID NO: 333 and a VH of SEQ ID NO: 335; or a VL of SEQ ID NO: 337 and a VH of SEQ ID NO: 339; or a VL of SEQ ID NO: 341 and a VH of SEQ ID NO: 343; or a VL of SEQ ID NO: 345 and a VH of SEQ ID NO: 347; or a VL of SEQ ID NO: 349 and a VH of SEQ ID NO: 351; or a VL of SEQ ID NO: 353 and a VH of SEQ ID NO: 355; or a VL of SEQ ID NO: 357 and a VH of SEQ ID NO: 359; or a VL of SEQ ID NO: 361 and a VH of SEQ ID NO: 363; or a VL of SEQ ID NO: 365 and a VH of SEQ ID NO: 367; or a VL of SEQ ID NO: 369 and a VH of SEQ ID NO: 371; or a VL of SEQ ID NO: 373 and a VH of SEQ ID NO: 375; or a VL of SEQ ID NO: 377 and a VH of SEQ ID NO: 379; or a VL of SEQ ID NO: 381 and a VH of SEQ ID NO: 383; or a VL of SEQ ID NO: 385 and a VH of SEQ ID NO: 387; or a VL of SEQ ID NO: 389 and a VH of SEQ ID NO: 391; or a VL of SEQ ID NO: 393 and a VH of SEQ ID NO: 395; or a VL of SEQ ID NO: 397 and a VH of SEQ ID NO: 399; or a VL of SEQ ID NO: 401 and a VH of SEQ ID NO: 403; or a VL of SEQ ID NO: 405 and a VH of SEQ ID NO: 407. In particularly preferred embodiments the DLL3 binding domain will comprise a scFv construct comprising the aforementioned VL and VH sequences or fragments thereof. In some aspects of the invention the CAR binding domain comprises a chimeric, CDR grafted, humanized or human antibody or an immunoreactive fragment thereof. In other aspects of the invention the CAR binding domain comprising the aforementioned sequences is an internalizing antibody.

Yet other preferred DLL3 CARs of the instant invention will comprise CDR grafted or humanized antibodies, or fragments or constructs thereof, comprising one or more heavy (CDRH1, CDRH2, CDRH3) or light (CDRL1, CDRL2, CDRL3) chain CDRs as set forth in FIG. 1A or 1B wherein the CDRs are derived as per Kabat et al.

In yet other compatible embodiments the CARs of the instant invention will comprise the binding region (e.g., in the form of a scFv) derived from one of the CDR grafted or humanized DLL3 antibodies hSC16.13, hSC16.15, hSC16.25, hSC16.34 and hSC16.56 or fragments thereof.

Other embodiments are directed to CARs comprising an antibody or antibody fragment or construct thereof wherein said antibody comprises:

an antibody light chain comprising a light chain variable region CDR1 comprising SEQ ID NO: 408, a light chain variable region CDR2 comprising SEQ ID NO: 409 and a light chain variable region CDR3 comprising SEQ ID NO: 410; and

an antibody heavy chain comprising a heavy chain variable region CDR1 comprising SEQ ID NO: 411, a heavy chain variable region CDR2 comprising SEQ ID NO: 412 and a heavy chain variable region CDR3 comprising SEQ ID NO: 413.

In another embodiment the invention is directed to CARs comprising an antibody or antibody fragment or construct thereof wherein said antibody comprises:

an antibody light chain comprising a light chain variable region CDR1 comprising SEQ ID NO: 414, a light chain variable region CDR2 comprising SEQ ID NO: 415 and a light chain variable region CDR3 comprising SEQ ID NO: 416; and

an antibody heavy chain comprising a heavy chain variable region CDR1 comprising SEQ ID NO: 417, a heavy chain variable region CDR2 comprising SEQ ID NO: 418 and a heavy chain variable region CDR3 comprising SEQ ID NO: 419.

In another embodiment the invention is directed to CARs comprising an antibody or antibody fragment or construct thereof wherein said antibody comprises:

an antibody light chain comprising a light chain variable region CDR1 comprising SEQ ID NO: 420, a light chain variable region CDR2 comprising SEQ ID NO: 421 and a light chain variable region CDR3 comprising SEQ ID NO: 422; and

an antibody heavy chain comprising a heavy chain variable region CDR1 comprising SEQ ID NO: 423, a heavy chain variable region CDR2 comprising SEQ ID NO: 424 and a heavy chain variable region CDR3 comprising SEQ ID NO: 425.

In another embodiment the invention is directed to CARs comprising an antibody or antibody fragment or construct thereof wherein said antibody comprises:

an antibody light chain comprising a light chain variable region CDR1 comprising SEQ ID NO: 426, a light chain variable region CDR2 comprising SEQ ID NO: 427 and a light chain variable region CDR3 comprising SEQ ID NO: 428; and

an antibody heavy chain comprising a heavy chain variable region CDR1 comprising SEQ ID NO: 429, a heavy chain variable region CDR2 comprising SEQ ID NO: 430 and a heavy chain variable region CDR3 comprising SEQ ID NO: 431.

In another embodiment the invention is directed to CARs comprising an antibody or antibody fragment or construct thereof wherein said antibody comprises:

an antibody light chain comprising a light chain variable region CDR1 comprising SEQ ID NO: 432, a light chain variable region CDR2 comprising SEQ ID NO: 433 and a light chain variable region CDR3 comprising SEQ ID NO: 434; and

an antibody heavy chain comprising a heavy chain variable region CDR1 comprising SEQ ID NO: 435, a heavy chain variable region CDR2 comprising SEQ ID NO: 436 and a heavy chain variable region CDR3 comprising SEQ ID NO: 437.

In certain preferred embodiments each of the aforementioned antibodies comprises humanized antibodies. Moreover, as described herein nucleic acid sequences encoding such exemplary murine and humanized heavy and light chain variable regions are set forth in the attached sequence listing.

In other embodiments the CARs of the present invention comprise an antibody or antibody fragment or construct thereof residing in a bin defined by a reference antibody selected from the group consisting of SC16.3, SC16.4, SC16.5, SC16.7, SC16.8, SC16.10, SC16.11, SC16.13, SC16.15, SC16.18, SC16.19, SC16.20, SC16.21, SC16.22, SC16.23, SC16.25, SC16.26, SC16.29, SC16.30, SC16.31, SC16.34, SC16.35, SC16.36, SC16.38, SC16.41, SC16.42, SC16.45, SC16.47, SC16.49, SC16.50, SC16.52, SC16.55, SC16.56, SC16.57, SC16.58, SC16.61, SC16.62, SC16.63, SC16.65, SC16.67, SC16.68, SC16.72, SC16.73, SC16.78, SC16.79, SC16.80, SC16.81, SC16.84, SC16.88, SC16.101, SC16.103, SC16.104, SC16.105, SC16.106, SC16.107, SC16.108, SC16.109, SC16.110, SC16.111, SC16.113, SC16.114, SC16.115, SC16.116, SC16.117, SC16.118, SC16.120, SC16.121, SC16.122, SC16.123, SC16.124, SC16.125, SC16.126, SC16.129, SC16.130, SC16.131, SC16.132, SC16.133, SC16.134, SC16.135, SC16.136, SC16.137, SC16.138, SC16.139, SC16.140, SC16.141, SC16.142, SC16.143, SC16.144, SC16.147, SC16.148, SC16.149 and SC16.150. In still other embodiments the CARs of the invention will comprise antibodies (or antibody fragments) from bin A, antibodies from bin B, antibodies from bin C, antibodies from bin D, antibodies from bin E, antibodies from bin F, antibodies from bin G, antibodies from bin H or antibodies from bin I. Yet other preferred embodiments will comprise a reference antibody and any antibody that competes with the reference antibody.

The term “compete” or “competing antibody” when used in the context of the disclosed binding domains means binding competition between antibodies as determined by an assay in which a reference antibody or immunoreactive fragment thereof substantially prevents or inhibits (e.g., greater than 30%, 35%, 40%, 45%, 50%, 55%, 60%, 65%, 70%, 75%, 80%, 85% or 90%) specific binding of a test antibody to a common antigen. Compatible methods for determining such competition comprise art known techniques such as, for example, bio-layer interferometry, surface plasmon resonance, flow cytometry, competitive ELISA, etc.

In certain embodiments the invention is directed to a nucleic acid encoding the heavy or light chain amino acid sequence (or constructs or derivatives thereof) of any one of the anti-DLL3 binding domains disclosed herein. Compatible anti-DLL3 heavy and light chain variable region nucleic acid sequences are set forth in the appended sequence listing. In preferred embodiments the nucleic acid encoding the binding domain or CAR are incorporated in a plasmid or vector. In yet other embodiments the vector will comprise a viral vector.

In another embodiment the present invention provides methods of treating cancer such as, for example, pancreatic cancer, colorectal cancer, prostate cancer, small cell and non-small cell lung cancer, breast cancer, ovarian cancer and gastric cancer, comprising administering a pharmaceutical composition comprising a host cell expressing an anti-DLL3 CAR as disclosed herein.

In some embodiments, the invention provides methods of treating cancer comprising administering a pharmaceutical composition comprising a host cell expressing an anti-DLL3 CAR as disclosed herein and further comprising administering to the subject at least one additional therapeutic moiety. In preferred embodiments the host cell will comprise a sensitized lymphocyte.

The present invention further provides a method of reducing tumor initiating cells in a tumor cell population, wherein the method comprises contacting a tumor cell population comprising tumor initiating cells and tumor cells other than tumor initiating cells, with a host cell expressing an anti-DLL3 CAR; whereby the frequency of tumor initiating cells is reduced.

In yet other preferred embodiments the present invention also provides kits or devices and associated methods that are useful in the treatment of DLL3 associated disorders such as cancer. To this end the present invention preferably provides an article of manufacture useful for generating DLL3 sensitized lymphocytes for treating DLL3 associated disorders comprising, for example, a container or receptacle containing vectors (e.g., viral vectors) encoding the disclosed CARs and instructional materials for generating DLL3 sensitized lymphocytes. In selected embodiments the kits will comprise additional reagents and receptacles to effectively transduce the lymphocytes. In other selected embodiments such kits comprise allogeneic DLL3 sensitized lymphocytes that may be directly administered to the patient to generate the desired immune response. In still other embodiments such articles of manufacture will comprise a container or receptacle comprising a liquid formulation of DLL3 sensitized lymphocytes. In such embodiments the DLL3 sensitized lymphocytes may comprise allogenic or autologous host cells and in other embodiments the liquid formulation may comprise a pharmaceutically acceptable carrier.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE FIGURES

FIGS. 1A and 1B provide, in a tabular form, contiguous amino acid sequences (SEQ ID NOS: 21-407, odd numbers) of light and heavy chain variable regions of a number of murine and humanized exemplary DLL3 antibodies compatible with the disclosed DLL3 CARs as isolated, cloned and engineered as described in the Examples herein;

FIG. 2 depicts, in schematic form, the results of domain level mapping analysis of exemplary DLL3 antibodies isolated, cloned and engineered as described in the Examples herein;

FIG. 3 provides a schematic representation of an exemplary DLL3 CAR construct illustrating the various components thereof;

FIGS. 4A-4C provide nucleic acid sequences and amino acid sequences for three exemplary DLL3 CARs (SCT1-h16.15, SCT1-h16.13 and SCT1-h16.25 respectively) compatible with the instant invention;

FIG. 5 provides a schematic representation illustrating a process for producing DLL3 sensitized lymphocytes and their subsequent use to generate an immune response directed to DLL3 positive tumor cells;

FIGS. 6A and 6B demonstrate the expression of exemplary DLL3 CARs (SCT1-h16.13, SCT1-h16.15 and SCT1-h16.25) on transduced Jurkat cells (FIG. 6A) and the expression of hDLL3 on engineered HEK-293T control cells (FIG. 6B) each as measured using flow cytometry;

FIGS. 7A and 7B depict the induction of an immune response in SCT1-h16.15 transduced Jurkat cells at various ratios of lymphocyte to target cells as measured by IL-2 production (FIG. 7A) and the induction of an immune response (again as measured by IL2 levels) generated using three different exemplary DLL3 CAR cells at the same lymphocyte to target cell ratio (FIG. 7B);

FIG. 8 demonstrates that human primary lymphocytes may be engineered to effectively express exemplary anti-DLL3 CARs in accordance with the teachings herein;

FIGS. 9A and 9B provide DLL3 surface expression profiles for a 293T cell line engineered to express DLL3 (FIG. 9A) and a small cell lung cancer patient derived xenograft (“PDX”) cell line (FIG. 9B) as evidenced by flow cytometry;

FIG. 10 shows the ability of DLL3 sensitized primary lymphocytes comprising three different DLL3 CARs (SCT1-h16.13, SCT1-h16.15 and SCT1-h16.25) to eliminate engineered 293T cells expressing DLL3;

FIG. 11 demonstrates that primary human lymphocytes from two individuals may be engineered to effectively express an anti-DLL3 CAR in accordance with the teachings herein;

FIGS. 12A and 12B demonstrate the ability of DLL3 sensitized lymphocytes comprising host cells from two individuals to provoke an immune response (as measured by the induction of TNFα) when exposed to engineered 293T cells (FIG. 12A) or PDX tumor cells (FIG. 12B);

FIGS. 13A and 13B demonstrate the ability of DLL3 sensitized lymphocytes comprising host cells from two individuals to provoke an immune response (as measured by the induction of INFγ) when exposed to engineered 293T cells (FIG. 13A) or PDX tumor cells (FIG. 13B); and

FIGS. 14A and 14B show the ability of DLL3 sensitized lymphocytes comprising host cells from two individuals to eliminate engineered 293T cells (FIG. 14A) or PDX tumor cells (FIG. 14B) upon exposure.

### DETAILED DESCRIPTION OF THE INVENTION

The invention may be embodied in many different forms. Disclosed herein are non-limiting, illustrative embodiments of the invention that exemplify the principles thereof. Any section headings used herein are for organizational purposes only and are not to be construed as limiting the subject matter described. For the purposes of the instant disclosure all identifying sequence accession numbers may be found in the NCBI Reference Sequence (RefSeq) database and/or the NCBI GenBank® archival sequence database unless otherwise noted.

Recent advances in adoptive transfer immunotherapy have provided a promising approach for the treatment of various neoplasia and a chance to improve patient experiences, particularly with regard to solid tumors. In this regard the present invention is directed to the use of novel chimeric antigen receptors (“CARs”) comprising an extracellular binding or targeting domain that associates or reacts with delta-like ligand 3 (“DLL3”). As will be discussed extensively herein, DLL3 is a particularly effective tumor marker that is expressed on a number of different cancers and, significantly, has been found to be associated with cancer stem cells. Thus, when the anti-DLL3 binding domains of the instant invention are incorporated in a chimeric antigen receptor expressed on lymphocytes, the resulting “DLL3 sensitized lymphocytes” (e.g., natural killer cells or T cells that immunospecifically recognize a DLL3 determinant) are able to effectively mount an immune response directed to aberrant DLL3 positive cells including cancer stem cells. This ability to effectively eliminate tumorigenic “seed” cells is often critical in reducing the possibility of tumor recurrence or metastasis. To this end it will be appreciated that the anti-DLL3 CAR lymphocytes of the instant invention may be used in combination with other therapeutic agents (including anti-DLL3 antibody drug conjugates) or as part of a maintenance regimen following standard of care treatments.

More generally a chimeric antigen receptor is an artificially constructed hybrid protein or polypeptide containing or comprising an antigen binding domain of an antibody linked to a signaling domain (e.g., T-cell signaling or T-cell activation domains). The CARs of the instant invention have the ability to redirect the specificity and reactivity of sensitized lymphocytes (e.g., T-cells) toward DLL3 positive target cells in a non-MHC-restricted manner by exploiting the antigen-binding properties of monoclonal antibodies. The non-MHC-restricted antigen recognition gives T-cells expressing DLL3 CARs the ability to recognize tumorigenic DLL3 independent of antigen processing, thus bypassing a major mechanism of tumor escape. Moreover, when expressed in T-cells, CARs advantageously do not dimerize with endogenous T cell receptor (TCR) alpha and beta chains.

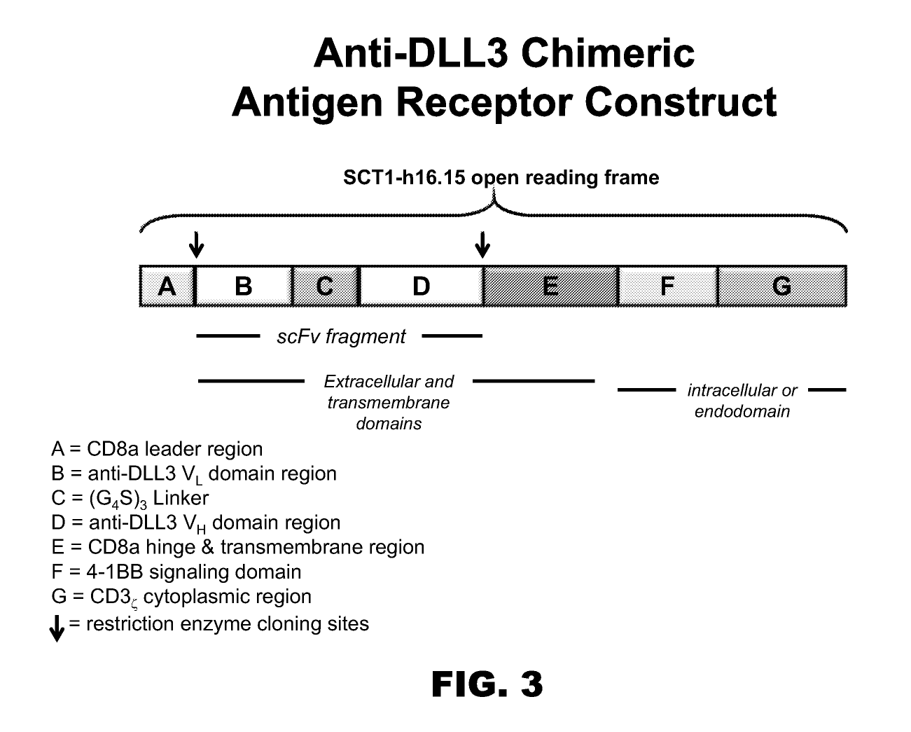

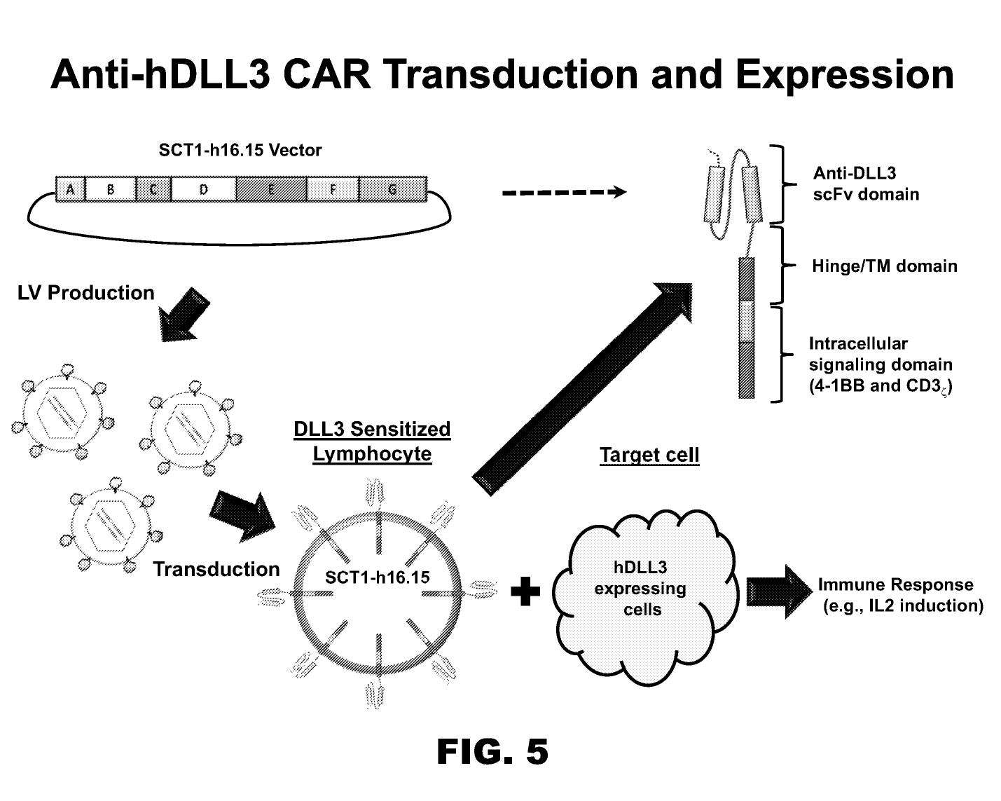

Accordingly, the present invention is generally directed to chimeric antigen receptors comprising a DLL3 binding domain that immunospecifically associates with DLL3 on target cells and stimulates an immune response. In preferred embodiments the DLL3 binding domain of the CAR may comprise a scFv derived from the heavy and light chain antibody variable regions disclosed herein. More specifically an “anti-DLL3 CAR” or simply “DLL3 CAR” of the instant invention shall comprise a chimeric protein incorporating an extracellular DLL3 binding domain, a transmembrane domain and an intracellular signaling domain (see FIG. 3). Typically a nucleotide sequence encoding the desired DLL3 CAR will be synthesized or engineered and inserted into an expression vector or system (e.g. lentiviral, retroviral, etc.). In preferred embodiments lymphocytes, including T-lymphocytes, natural killer cells (“NK cells”) and dendritic cells, obtained from a patient or donor are then exposed to (e.g., transduced) the selected DLL3 CAR vector to provide engineered lymphocytes that express the CAR protein with the extracellular DLL3 binding domain (i.e., “DLL3 sensitized lymphocytes”). Following optional expansion, these DLL3 sensitized lymphocytes may be infused into a patient to mount an immunospecific response to DLL3 positive tumor cells (see generally FIG. 5). In this regard the DLL3 sensitized lymphocytes will be activated upon contacting a target cell expressing a DLL3 determinant. To “activate the sensitized lymphocytes” (e.g., T cells and NK cells) means to induce a change in their biologic state by which the cells express activation markers, produce cytokines, proliferate and/or become cytotoxic to target cells. All these changes can be produced by primary stimulatory signals. Costimulatory signals amplify the magnitude of the primary signals and suppress cell death following initial stimulation resulting in a more durable activation state and thus a higher cytotoxic capacity.

Thus, it will further be appreciated that, besides the DLL3 binding domain, CARs of the invention will comprise an intracellular or cytoplasmic domain that initiates a primary cytoplasmic signaling sequence (e.g., a sequence for initiating antigen-dependent primary activation via a T-cell receptor complex). Compatible intracellular domains may, for example, be derived from CD3ζ, FcRγ, FcRβ, CD3γ, CD3δ, CD3ε, CD5, CD22, CD79a, CD79b, and CD66d. In other preferred embodiments, the CARs of the invention will comprise an intracellular domain that initiates a secondary or co-stimulating signal. Compatible costimulatory domains may comprise, for example, intracellular domains derived from CD2, CD4, CD5, CD8α, CD8β, CD28, CD134, CD137, ICOS, CD154, 4-1BB and glucocorticoid-induced tumor necrosis factor receptor (see U.S.P.N. US/2014/0242701). Additionally, in preferred embodiments the disclosed CARs will comprise a transmembrane (and optionally a spacer) domain interposed between the extracellular DLL3 binding domain and the intracellular signaling domain. As discussed in more detail below the transmembrane domain may comprise, for example, part of an antibody constant (Fc) region, human CD8a or artificially produced spacers known in the art. Essentially, any amino acid sequence that anchors the CAR in the cell membrane and allows for effective association of the DLL3 binding domain and transmission of appropriate signaling from the intracellular domain is compatible with the invention.

With respect to the novel DLL3 CARs of the invention, it will be appreciated that the selection of DLL3 as the tumor target is integral in generating an effective anti-tumor immune response. More specifically it has been found that DLL3 phenotypic determinants are clinically associated with various proliferative disorders, including neoplasia exhibiting neuroendocrine features, and that DLL3 protein and variants or isoforms thereof provide useful tumor markers which may be exploited in the treatment of related diseases. In this regard the present invention provides a number of chimeric antigen receptors that comprise an anti-DLL3 binding domain in addition to any signaling component. As discussed in more detail below, the disclosed DLL3 CARs are particularly effective at eliminating tumorigenic cells and therefore useful for the treatment and prophylaxis of certain proliferative disorders or the progression or recurrence thereof.

Moreover, as shown in the instant application it has been found that DLL3 markers or determinants such as cell surface DLL3 protein are therapeutically associated with cancer stem cells (also known as tumor perpetuating cells) and may be effectively leveraged to eliminate or silence the same. The ability to selectively reduce or eliminate cancer stem cells through the use of DLL3 CARs as disclosed herein is surprising in that such cells are known to generally be resistant to many conventional treatments. That is, the effectiveness of traditional, as well as more recent targeted treatment methods, is often limited by the existence and/or emergence of resistant cancer stem cells that are capable of perpetuating tumor growth even in face of diverse treatment methods. Further, determinants associated with cancer stem cells often make poor therapeutic targets due to low or inconsistent expression, failure to remain associated with the tumorigenic cell or failure to present at the cell surface. In sharp contrast to the teachings of the prior art, the instantly disclosed DLL3 CARs and associated methods effectively overcome this inherent resistance to specifically eliminate, deplete, silence or promote the differentiation of such cancer stem cells thereby negating their ability to sustain or, significantly, re-induce underlying tumor growth. Moreover, as expression of DLL3 protein has largely been associated with intracellular locations such as the Golgi, it was uncertain that such phenotypic determinants could be successfully exploited as a therapeutic target for the specific DLL3 CARs as taught herein.

Thus, it is particularly remarkable that DLL3 CARs such as those disclosed herein may advantageously be used in the treatment and/or prevention of selected proliferative (e.g., neoplastic) disorders or progression or recurrence thereof. It will be appreciated that, while preferred embodiments of the invention will be discussed extensively below, particularly in terms of exemplary signaling or costimulatory domains or regions or in the context of cancer stem cells or tumors comprising neuroendocrine features and their interactions with the disclosed DLL3 CARs, those skilled in the art will appreciate that the scope of the instant invention is not limited by such exemplary embodiments. Rather, the most expansive embodiments of the present invention and the appended claims are broadly and expressly directed to any chimeric antigen receptor comprising a binding domain that immunospecifically associates or binds to DLL3 and their use in the treatment and/or prevention of a variety of DLL3 associated or mediated disorders, including neoplastic or cell proliferative disorders, regardless of any particular mechanism of action, CAR construct or specifically targeted tumor, cellular or molecular component.

To that end, and as demonstrated in the instant application, it has unexpectedly been found that the disclosed DLL3 CARs can effectively be used to target and eliminate or otherwise incapacitate proliferative or tumorigenic cells and treat DLL3 associated disorders (e.g., neoplasia). As used herein a “DLL3 associated disorder” shall be held to mean any disorder or disease (including proliferative disorders) that is marked, diagnosed, detected or identified by a phenotypic aberration of DLL3 genetic components or expression (“DLL3 determinant”) during the course or etiology of the disease or disorder. In this regard a DLL3 phenotypic aberration or determinant may, for example, comprise elevated or depressed levels of DLL3 protein expression, abnormal DLL3 protein expression on certain definable cell populations or abnormal DLL3 protein expression at an inappropriate phase or stage of a cell lifecycle. Of course, it will be appreciated that similar expression patterns of genotypic determinants (e.g., mRNA transcription levels) of DLL3 may also be used to classify, detect or treat DLL3 disorders.

### I. DLL3 Physiology

It has been found that DLL3 phenotypic determinants are clinically associated with various proliferative disorders, including neoplasia exhibiting neuroendocrine features, and that DLL3 protein and variants or isoforms thereof provide useful tumor markers which may be exploited in the treatment of related diseases. In this regard the present invention provides a number of DLL3 CAR constructs comprising an engineered anti-DLL3 binding or targeting agent operably associated with one or more signaling domain(s) capable of inducing an immune response in a lymphocyte. As discussed in more detail below and set forth in the appended Examples, the disclosed anti-DLL3 CARs are particularly effective at eliminating tumorigenic cells and therefore useful for the treatment and prophylaxis of certain proliferative disorders or the progression or recurrence thereof.

Moreover, it has been found that DLL3 markers or determinants such as cell surface DLL3 protein are therapeutically associated with cancer stem cells (also known as tumor perpetuating cells) and may be effectively exploited to eliminate or silence the same. The ability to selectively reduce or eliminate cancer stem cells through the use of DLL3 CARs as disclosed herein is surprising in that such cells are known to generally be resistant to many conventional treatments. That is, the effectiveness of traditional, as well as more recent targeted treatment methods, is often limited by the existence and/or emergence of resistant cancer stem cells that are capable of perpetuating tumor growth even in face of these diverse treatment methods. Further, determinants associated with cancer stem cells often make poor therapeutic targets due to low or inconsistent expression, failure to remain associated with the tumorigenic cell or failure to present at the cell surface. In sharp contrast to the teachings of the prior art, the instantly disclosed CARs and methods effectively overcome this inherent resistance and to specifically eliminate, deplete, silence or promote the differentiation of such cancer stem cells thereby negating their ability to sustain or re-induce the underlying tumor growth.

In Drosophila, Notch signaling is mediated primarily by one Notch receptor gene and two ligand genes, known as Serrate and Delta (Wharton et al, 1985; Rebay et al., 1991). In humans, there are four known Notch receptors and five DSL (Delta-Serrate LAG2) ligands—two homologs of Serrate, known as Jagged1 and Jagged 2, and three homologs of Delta, termed delta-like ligands or DLL1, DLL3 and DLL4. In general, Notch receptors on the surface of the signal-receiving cell are activated by interactions with ligands expressed on the surface of an opposing, signal-sending cell (termed a trans-interaction). These trans-interactions lead to a sequence of protease mediated cleavages of the Notch receptor. In consequence, the Notch receptor intracellular domain is free to translocate from the membrane to the nucleus, where it partners with the CSL family of transcription factors (RBPJ in humans) and converts them from transcriptional repressors into activators of Notch responsive genes.

Of the human Notch ligands, DLL3 is different in that it seems incapable of activating the Notch receptor via trans-interactions (Ladi et al., 2005). Notch ligands may also interact with Notch receptors in cis (on the same cell) leading to inhibition of the Notch signal, although the exact mechanisms of cis-inhibition remain unclear and may vary depending upon the ligand (for instance, see Klein et al., 1997; Ladi et al., 2005; Glittenberg et al., 2006). Two hypothesized modes of inhibition include modulating Notch signaling at the cell surface by preventing trans-interactions, or by reducing the amount of Notch receptor on the surface of the cell by perturbing the processing of the receptor or by physically causing retention of the receptor in the endoplasmic reticulum or Golgi (Sakamoto et al., 2002; Dunwoodie, 2009). It is clear, however, that stochastic differences in expression of Notch receptors and ligands on neighboring cells can be amplified through both transcriptional and non-transcriptional processes, and subtle balances of cis- and trans-interactions can result in a fine tuning of the Notch mediated delineation of divergent cell fates in neighboring tissues (Sprinzak et al., 2010).

DLL3 is a member of the Delta-like family of Notch DSL ligands. Representative DLL3 protein orthologs include, but are not limited to, human (Accession Nos. NP_058637 and NP_982353), chimpanzee (Accession No. XP_003316395), mouse (Accession No. NP_031892), and rat (Accession No. NP_446118). In humans, the DLL3 gene consists of 8 exons spanning 9.5 kBp located on chromosome 19q13. Alternate splicing within the last exon gives rise to two processed transcripts, one of 2389 bases (Accession No. NM_016941) and one of 2052 bases (Accession No. NM_203486). The former transcript encodes a 618 amino acid protein (Accession No. NP_058637; SEQ ID NO: 1), whereas the latter encodes a 587 amino acid protein (Accession No. NP_982353; SEQ ID NO: 2). These two protein isoforms of DLL3 share overall 100% identity across their extracellular domains and their transmembrane domains, differing only in that the longer isoform contains an extended cytoplasmic tail containing 32 additional residues at the carboxy terminus of the protein. The biological relevance of the isoforms is unclear, although both isoforms can be detected in tumor cells.

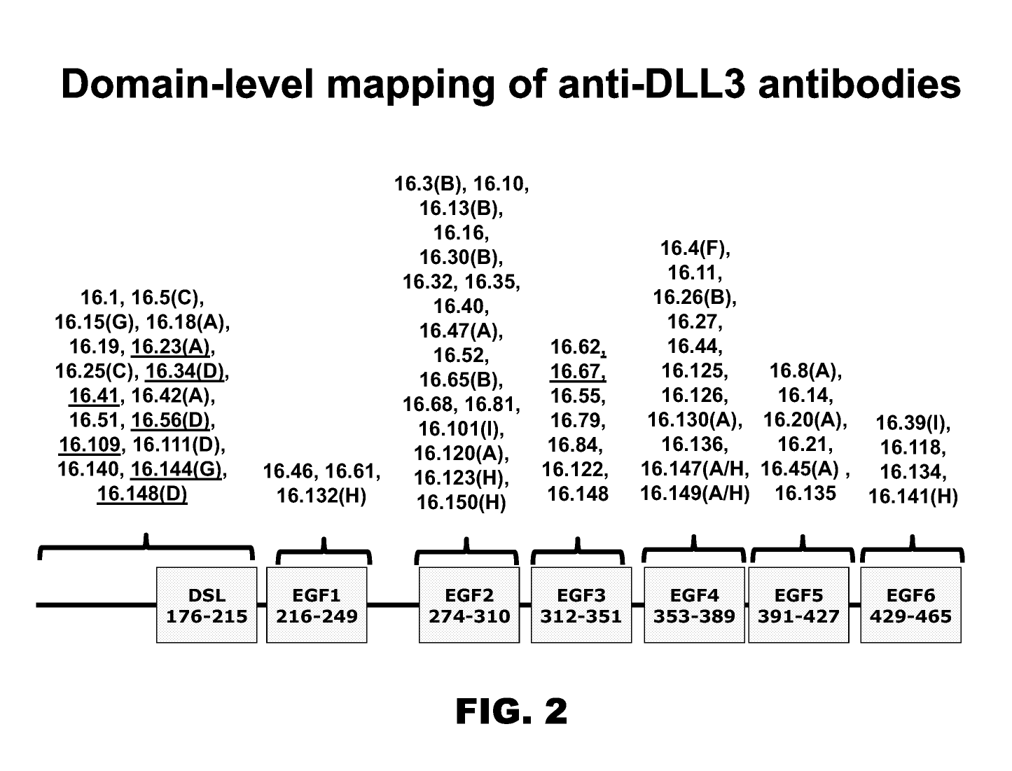

As shown schematically in FIG. 2 the extracellular region of the DLL3 protein, comprises six EGF-like domains, the single DSL domain and the N-terminal domain. Generally, the EGF domains are recognized as occurring at about amino acid residues 216-249 (domain 1), 274-310 (domain 2), 312-351 (domain 3), 353-389 (domain 4), 391-427 (domain 5) and 429-465 (domain 6), with the DSL domain at about amino acid residues 176-215 and the N-terminal domain at about amino acid residues 27-175 of hDLL3 (SEQ ID NOS: 1 and 2). As discussed in more detail herein and shown in the Examples below, each of the EGF-like domains, the DSL domain and the N-terminal domain comprise part of the DLL3 protein as defined by a distinct amino acid sequence. Note that, for the purposes of the instant disclosure the respective EGF-like domains may be termed EGF1 to EGF6 with EGF1 being closest to the N-terminal portion of the protein. In regard to the structural composition of the protein one significant aspect of the instant invention is that the disclosed DLL3 modulators may be generated, fabricated, engineered or selected so as to react with a selected domain, motif or epitope. In certain cases such site-specific modulators may provide enhanced reactivity and/or efficacy depending on their primary mode of action. In particularly preferred embodiments the DLL3 CAR will bind to the DSL domain and, in even more preferred embodiments, will bind to an epitope comprising G203, R205, P206 (SEQ ID NO: 4) within the DSL domain.

### II. Cancer Stem Cells

As alluded to above it has surprisingly been discovered that aberrant DLL3 expression (genotypic and/or phenotypic) is associated with various tumorigenic cell subpopulations. In this respect the present invention provides DLL3 CAR mediated therapeutic regimens that may be particularly useful for targeting such cells (e.g., cancer stem cells), thereby facilitating the treatment, management or prevention of neoplastic disorders. Thus, in preferred embodiments the disclosed DLL3 CAR may be advantageously be used to reduce tumor initiating cell frequency in accordance with the present teachings and thereby facilitate the treatment or management of proliferative disorders.

According to the current models, a tumor comprises non-tumorigenic cells and tumorigenic cells. Non-tumorigenic cells do not have the capacity to self-renew and are incapable of reproducibly forming tumors, even when transplanted into immunocompromised mice in excess cell numbers. Tumorigenic cells, also referred to herein as “tumor initiating cells” (TICs), which make up 0.1-40% (more typically 0.1-10%) of a tumor's cell population, have the ability to form tumors. Tumorigenic cells encompass both tumor perpetuating cells (TPCs), referred to interchangeably as cancer stem cells (CSCs) and tumor progenitor cells (TProgs).

CSCs, like normal stem cells that support cellular hierarchies in normal tissue, are able to self-replicate indefinitely while maintaining the capacity for multilineage differentiation. CSCs are able to generate both tumorigenic progeny and non-tumorigenic progeny and are able to completely recapitulate the heterogeneous cellular composition of the parental tumor as demonstrated by serial isolation and transplantation of low numbers of isolated CSCs into immunocompromised mice.

Tprogs, like CSCs have the ability to fuel tumor growth in a primary transplant. However, unlike CSCs, they are not able to recapitulate the cellular heterogeneity of the parental tumor and are less efficient at reinitiating tumorigenesis in subsequent transplants because Tprogs are typically only capable of a finite number of cell divisions as demonstrated by serial transplantation of low numbers of highly purified Tprog into immunocompromised mice. Tprogs may further be divided into early Tprogs and late Tprogs, which may be distinguished by phenotype (e.g., cell surface markers) and their different capacities to recapitulate tumor cell architecture. While neither can recapitulate a tumor to the same extent as CSCs, early Tprogs have a greater capacity to recapitulate the parental tumor's characteristics than late Tprogs. Notwithstanding the foregoing distinctions, it has been shown that some Tprog populations can, on rare occasion, gain self-renewal capabilities normally attributed to CSCs and can themselves become CSCs.

CSCs exhibit higher tumorigenicity and are relatively more quiescent than: (i) Tprogs (both early and late Tprogs); and (ii) non-tumorigenic cells such as tumor-infiltrating cells, for example, fibroblasts/stroma, endothelial and hematopoietic cells that may be derived from CSCs and typically comprise the bulk of a tumor. Given that conventional therapies and regimens have, in large part, been designed to debulk tumors and attack rapidly proliferating cells, CSCs are more resistant to conventional therapies and regimens than the faster proliferating Tprogs and other bulk tumor cell populations such as non-tumorigenic cells. Other characteristics that may make CSCs relatively chemoresistant to conventional therapies are increased expression of multi-drug resistance transporters, enhanced DNA repair mechanisms and anti-apoptotic gene expression. These properties in CSCs constitute a key reason for the failure of standard oncology treatment regimens to ensure long-term benefit for most patients with advanced stage neoplasia because standard chemotherapy does not target the CSCs that actually fuel continued tumor growth and recurrence.

It has surprisingly been discovered that DLL3 expression is associated with various tumorigenic cell populations. The invention provides DLL3 CARs that may be particularly useful for targeting tumorigenic cells and may be used to silence, sensitize, neutralize, reduce the frequency, block, abrogate, interfere with, decrease, hinder, restrain, control, deplete, moderate, mediate, diminish, reprogram, eliminate, or otherwise inhibit (collectively, “inhibit”) tumorigenic cells, thereby facilitating the treatment, management and/or prevention of proliferative disorders (e.g. cancer). Advantageously, the novel DLL3 CARs of the invention may be selected so they preferably reduce the frequency or tumorigenicity of tumorigenic cells upon administration to a subject regardless of the form of the DLL3 determinant (e.g., isotype a or b). The reduction in tumorigenic cell frequency may occur as a result of (i) inhibition or eradication of tumorigenic cells; (ii) controlling the growth, expansion or recurrence of tumorigenic cells; (iii) interrupting the initiation, propagation, maintenance, or proliferation of tumorigenic cells; or (iv) by otherwise hindering the survival, regeneration and/or metastasis of the tumorigenic cells. In some embodiments, the inhibition of tumorigenic cells may occur as a result of a change in one or more physiological pathways. The change in the pathway, whether by inhibition of the tumorigenic cells, modification of their potential (for example, by induced differentiation or niche disruption) or otherwise interfering with the ability of tumorigenic cells to influence the tumor environment or other cells, allows for the more effective treatment of DLL3 associated disorders by inhibiting tumorigenesis, tumor maintenance and/or metastasis and recurrence.

Methods that can be used to assess the reduction in the frequency of tumorigenic cells, include but are not limited to, cytometric or immunohistochemical analysis, preferably by in vitro or in vivo limiting dilution analysis (Dylla et al. 2008, PMID: PMC2413402 and Hoey et al. 2009, PMID: 19664991).

Flow cytometry and immunohistochemistry may also be used to determine tumorigenic cell frequency. Both techniques employ one or more antibodies or reagents that bind art recognized cell surface proteins or markers known to enrich for tumorigenic cells (see WO 2012/031280). As known in the art, flow cytometry (e.g. florescence activated cell sorting (FACS)) can also be used to characterize, isolate, purify, enrich or sort for various cell populations including tumorigenic cells. Flow cytometry measures tumorigenic cell levels by passing a stream of fluid, in which a mixed population of cells is suspended, through an electronic detection apparatus which is able to measure the physical and/or chemical characteristics of up to thousands of particles per second. Immunohistochemistry provides additional information in that it enables visualization of tumorigenic cells in situ (e.g., in a tissue section) by staining the tissue sample with labeled antibodies or reagents which bind to tumorigenic cell markers.

Listed below are markers that have been associated with CSC populations and have been used to isolate or characterize CSCs: ABCA1, ABCA3, ABCG2, DLL3, ADCY9, ADORA2A, AFP, AXIN1, B7H3, BCL9, Bmi-1, BMP-4, C20orf52, C4.4A, carboxypeptidase M, CAV1, CAV2, CD105, CD133, CD14, CD16, CD166, CD16a, CD16b, CD2, CD20, CD24, CD29, CD3, CD31, CD324, CD325, CD3ζ, CD38, CD44, CD45, CD46, CD49b, CD49f, CD56, CD64, CD74, CD9, CD90, CEACAM6, CELSR1, CPD, CRIM1, CX3CL1, CXCR4, DAF, decorin, easyh1, easyh2, EDG3, eed, EGFR, ENPP1, EPCAM, EPHA1, EPHA2, FLJ10052, FLVCR, FZD1, FZD10, FZD2, FZD3, FZD4, FZD6, FZD7, FZD8, FZD9, GD2, GJA1, GLI1, GLI2, GPNMB, GPR54, GPRC5B, IL1R1, IL1RAP, JAM3, Lgr5, Lgr6, LRP3, LY6E, MCP, mf2, mllt3, MPZL1, MUC1, MUC16, MYC, N33, Nanog, NB84, nestin, NID2, NMA, NPC1, oncostatin M, OCT4, OPN3, PCDH7, PCDHA10, PCDHB2, PPAP2C, PTPN3, PTS, RARRES1, SEMA4B, SLC19A2, SLC1A1, SLC39A1, SLC4A11, SLC6A14, SLC7A8, smarcA3, smarcD3, smarcE1, smarcA5, Sox1, STAT3, STEAP, TCF4, TEM8, TGFBR3, TMEPAI, TMPRSS4, transferrin receptor, TrkA, WNT10B, WNT16, WNT2, WNT2B, WNT3, WNT5A, YY1 and 1-catenin. See, for example, Schulenburg et al., 2010, PMID: 20185329, U.S.P.N. 7,632,678 and U.S.P.N.s. 2007/0292414, 2008/0175870, 2010/0275280, 2010/0162416 and 2011/0020221.

Similarly, non-limiting examples of cell surface phenotypes associated with CSCs of certain tumor types include CD44^{hi}CD24^{low}, ALDH⁺, CD133⁺, CD123⁺, CD34⁺CD38⁻, CD44⁺CD24⁻, CD46^{hi}CD324⁺CD66c⁻, CD133⁺CD34⁺CD10⁻CD19⁻, CD138⁻CD34⁻CD19⁺, CD133⁺RC2⁺, CD44⁺α₂β₁^{hi}CD133⁺, CD44⁺CD24⁺ESA⁺, CD271⁺, ABCB5⁺ as well as other CSC surface phenotypes that are known in the art. See, for example, Schulenburg et al., 2010, supra, Visvader et al., 2008, PMID: 18784658 and U.S.P.N. 2008/0138313. Of particular interest with respect to the instant invention are CSC preparations comprising CD46^{hi}CD324⁺ phenotypes.

“Positive,” “low” and “negative” expression levels as they apply to markers or marker phenotypes are defined as follows. Cells with negative expression (i.e. “−”) are herein defined as those cells expressing less than, or equal to, the 95^{th} percentile of expression observed with an isotype control antibody in the channel of fluorescence in the presence of the complete antibody staining cocktail labeling for other proteins of interest in additional channels of fluorescence emission. Those skilled in the art will appreciate that this procedure for defining negative events is referred to as “fluorescence minus one”, or “FMO”, staining. Cells with expression greater than the 95^{th} percentile of expression observed with an isotype control antibody using the FMO staining procedure described above are herein defined as “positive” (i.e. “+”). As defined herein there are various populations of cells broadly defined as “positive.” A cell is defined as positive if the mean observed expression of the antigen is above the 95^{th} percentile determined using FMO staining with an isotype control antibody as described above. The positive cells may be termed cells with low expression (i.e. “lo”) if the mean observed expression is above the 95^{th} percentile determined by FMO staining and is within one standard deviation of the 95^{th} percentile. Alternatively, the positive cells may be termed cells with high expression (i.e. “hi”) if the mean observed expression is above the 95^{th} percentile determined by FMO staining and greater than one standard deviation above the 95^{th} percentile. In other embodiments the 99^{th} percentile may preferably be used as a demarcation point between negative and positive FMO staining and in particularly preferred embodiments the percentile may be greater than 99%.

The CD46^{hi}CD324⁺ marker phenotype and those exemplified immediately above may be used in conjunction with standard flow cytometric analysis and cell sorting techniques to characterize, isolate, purify or enrich TIC and/or TPC cells or cell populations for further analysis.

The ability of the CARs of the current invention to reduce the frequency of tumorigenic cells can therefore be determined using the techniques and markers described above. In some instances DLL3 CAR may reduce the frequency of tumorigenic cells by 10%, 15%, 20%, 25%, 30% or even by 35%. In other embodiments, the reduction in frequency of tumorigenic cells may be in the order of 40%0, 45%, 50%, 55%, 60% or 65%. In certain embodiments, the disclosed adoptive immunotherapy may reduce the frequency of tumorigenic cells by 70%, 75%, 80%, 85%, 90% or even 95%. It will be appreciated that any reduction of the frequency of tumorigenic cells is likely to result in a corresponding reduction in the tumorigenicity, persistence, recurrence and aggressiveness of the neoplasia.

### III. Chimeric Antigen Receptor Therapy

Cancer immunotherapies aim to harness the power of the human immune system to eradicate tumors via the activity of cytotoxic lymphocytes (comprising both cytotoxic T-lymphocytes and NK cells). That cytotoxic lymphocyte-mediated immune responses could lead to the eradication of residual tumor cells was inferred from studies that compared relapse rates in leukemia patients that had undergone various types of transplantation: a significant reduction in relapse rates was observed for patients receiving non-T-cell depleted marrow in allogeneic transplants from HLA identical siblings versus those receiving syngenic transplants, and this effect could be attributed to other T-cell mediated actions beyond graft-versus-host disease responses. However, clinically effective adoptive transfer of anti-tumor T-cells has been hampered by the fact that most tumor antigens are self-antigens, and therefore are poorly immunogenic. More specifically negative selection of T-cells bearing high-affinity T-cell receptors (TCRs) recognizing self-antigens takes place in the thymus during development, resulting in central tolerance and selection for T-cells having low-avidity recognition of the tumor/self-antigen. These lower avidity T-cells then have consequent weak activation of anti-tumor T-cell function and limited persistence. Genetically engineered cytotoxic lymphocytes are being deployed in two major approaches to circumvent this tolerance/low avidity block to strong anti-tumor T-cell activation. In the first approach, affinity-enhanced TCR recognizing tumor antigens are artificially introduced into T-cells using molecular genetic engineering techniques. This approach is limited by several factors, including difficulty in expressing the affinity-enhanced TCR at levels approaching wild-type TCR expression, the potential for mispairing of TCR chains which arises when introducing additional sets of TCR genes into a native T-cell, and the ability of tumor cells to evade MHC-restricted TCR recognition by down-regulating MHC molecules.

A second approach to the genetic engineering of cytotoxic lymphocytes is introduction of an artificial, non-MHC-restricted chimeric antigen receptor (CAR) into various lymphocyte populations. This is most typically achieved by harvesting bulk lymphocyte populations which are cultured, stimulated and expanded ex vivo prior to transduction with retroviral or lentiviral vectors encoding the CAR molecule. Like a native TCR, the CAR must possess the ability to specifically and selectively recognize a target antigen, and then upon binding to this antigen, transduce the appropriate signals to the lymphocyte to stimulate effector functions and/or the cytokine production necessary for a sustained anti-tumor immunological response. The concept of CAR-modified T-cells arose from studies in which it was observed that the cytoplasmic ITAM domain of the CD3ζ chain could activate T-cells when expressed independently from the TCR:CD3 protein complex, particularly when the CD3ζ ITAM domain was fused to a heterologous extracellular and transmembrane domain. A first-generation CD4-CD3ζ CAR was transduced into T-cells and tested in HIV patients. Follow up studies showed these engineered CAR-T cells persisted for up to a decade after infusion, indicative of some proliferation and persistence of the engineered cells. Subsequently, anti-tumor CARs were constructed by combining in a single recombinant molecule a scFv domain and a transmembrane domain with the cytoplasmic domain of the CD3ζ chain, and it could be shown the antigen recognition of these engineered CAR-T cells was redirected to reflect the specificity of the scFv (U.S.P.N. 7,446,179). These first generation scFv-directed CAR-T cells were capable of acting as non-MHC restricted cytotoxic lymphocytes, recognizing native tumor antigen rather than processed peptides, and promoting lysis of tumor cells expressing the native antigen.

While many of the first generation scFv-directed CAR-T cells showed expected effects in vitro, in vivo studies in cancer patients were disappointing for their lack of anti-tumor effects and lack of CAR-T persistence. As T-cell biology has become better understood, it has become clear that T-cell populations are comprised of short lived effector cells, longer-lived central and peripheral memory T-cells, as well as regulatory T-cells (Tregs) that interact with the other T-cell subpopulations. Central to function of these populations are the role of costimulatory signals in inducing persistent activation of resting naïve or memory T-cells via cytokine production, as well as the role co-stimulation provides in preventing anergy, a state of T-cell non-responsiveness that may potentially arise from exclusive TCR:CD3ζ signaling in the absence of costimulatory signals. In particular, various costimulatory signals from proteins such as CD28, OX40, CD27, CD137/4-1BB, CD2, CD3, CD11a/CD18, CD54 and CD58 may be beneficial for optimal levels of cytokine production, proliferation and clonal expansion, and induction of cytolytic activity. Of these, CD28 is perhaps the best understood costimulatory signal, and CD28 co-stimulation has been shown to augment cytokine release by antigen activated CAR-T cells. Similarly, costimulatory signaling through CD137/4-1BB has been shown to enhance native T-cell proliferation, and may contribute to longer persistence of CAR-T in vivo. Therefore, so called second generation CAR constructs have been designed in which various additional signaling domains from these molecules have been added in tandem to the CD3ζ domain (U.S.P.N.s. 5,686,281 and 8,399,645). So called third generation CAR molecules including three or more signaling domains (e.g., CD3ζ and two costimulatory signaling domains) are also reportedly under development.

Several second generation CAR-T cells directed to the CD19 antigen have been shown to have strong antitumor effects, as well as substantial persistence, in patients with hematological malignancies. To date, the effectiveness of CAR-T therapies with respect to the treatment of solid tumors remains to be conclusively demonstrated. With respect to the instant invention it has surprisingly been discovered that anti-DLL3 binding domains may be advantageously integrated with each of the aforementioned chimeric antigen receptors and adoptive immunotherapies to provide effective antineoplastic treatments that overcome some of the previous limitations.

### IV. Chimeric Antigen Receptors

As alluded to above the CARs of the instant invention generally comprise an extracellular domain comprising a DLL3 binding domain, a transmembrane domain and an intracellular signaling domain that activates certain lymphocytes and generates an immune response directed to DLL3 positive tumor cells. More generally, the disclosed chimeric antigen receptors comprise an ectodomain and an endodomain each as defined by the host cell wall. In this regard the terms “ectodomain” or “extracellular domain” will refer to the portion of the CAR polypeptide outside of the cell or exterior to the membranous lipid bilayer, which may comprise the antigen recognition (e.g., DLL3) binding domains, an optional hinge region, and any spacer domains exterior the to the amino acid residues physically spanning the membrane. Conversely the terms “endodomain” or “intracellular domain” will refer to the portion of the CAR polypeptide inside the cell or interior to the membranous lipid bilayer, which may comprise any spacer domains interior to the amino acid residues physically spanning the membrane, as well as the intracellular signaling domain.

A. DLL3 Binding Domains

1. Binding Domain Structure

As discussed extensively throughout the instant disclosure, chimeric antigen receptors comprising an anti-DLL3 binding domain may advantageously be used to provide targeted therapies for various proliferative disorders. It will be appreciated that compatible anti-DLL3 binding domains may comprise anti-DLL3 antibodies or immunoreactive fragments or constructs or derivatives thereof. In certain embodiments intact antibodies or antibodies comprising at least some portion of the fc or constant domain comprise the DLL3 binding domain (see, for example, U.S.P.N. 2015/0139943). In other preferred embodiments, and as demonstrated in the Examples appended hereto, the anti-DLL3 binding domain may comprise a scFv derived from a monoclonal antibody (including humanized or CDR grafted monoclonal antibodies) that binds to DLL3. Compatible antibodies that may be used to provide DLL3 binding domains consistent with the instant invention are discussed in more detail immediately below. For the purposes of the instant application the terms “binding domain” and “antibody” may be used interchangeably unless otherwise contextually dictated.

Antibodies and variants and derivatives thereof, including accepted nomenclature and numbering systems, have been extensively described, for example, in Abbas et al. (2010), Cellular and Molecular Immunology (6^{th} Ed.), W.B. Saunders Company; or Murphey et al. (2011), Janeway's Immunobiology (8 Ed.), Garland Science.

An “intact antibody” typically comprises a Y-shaped tetrameric protein comprising two heavy (H) and two light (L) polypeptide chains held together by covalent disulfide bonds and non-covalent interactions. Each light chain is composed of one variable domain (VL) and one constant domain (CL). Each heavy chain comprises one variable domain (VH) and a constant region, which in the case of IgG, IgA, and IgD antibodies, comprises three domains termed CH1, CH2, and CH3 (IgM and IgE have a fourth domain, CH4). In IgG, IgA, and IgD classes the CH1 and CH2 domains are separated by a flexible hinge region, which is a proline and cysteine rich segment of variable length (from about 10 to about 60 amino acids in various IgG subclasses). The variable domains in both the light and heavy chains are joined to the constant domains by a “J” region of about 12 or more amino acids and the heavy chain also has a “D” region of about 10 additional amino acids. Each class of antibody further comprises inter-chain and intra-chain disulfide bonds formed by paired cysteine residues.

As alluded to above the term “antibody” should be construed generally and includes polyclonal antibodies, monoclonal antibodies, chimeric antibodies, humanized and primatized antibodies, CDR grafted antibodies, human antibodies, recombinantly produced antibodies, intrabodies, multispecific antibodies, bispecific antibodies, monovalent antibodies, multivalent antibodies, anti-idiotypic antibodies, synthetic antibodies, including muteins and variants thereof, immunospecific antibody fragments such as Fd, Fab, F(ab′)₂, F(ab′) fragments, single-chain fragments (e.g. scFv and ScFvFc); and derivatives thereof including Fc fusions and other modifications, and any other immunoreactive immunoglobulin molecule so long as it exhibits preferential association or binding with a DLL3 determinant. Moreover, unless dictated otherwise by contextual constraints the term further comprises all classes of antibodies (i.e. IgA, IgD, IgE, IgG, and IgM) and all subclasses (i.e., IgG1, IgG2, IgG3, IgG4, IgA1, and IgA2) and all immunoreactive fragments thereof. Heavy-chain constant domains that correspond to the different classes of antibodies are typically denoted by the corresponding lower case Greek letter α, δ, ε, γ, and μ, respectively. Light chains of the antibodies from any vertebrate species can be assigned to one of two clearly distinct types, called kappa (κ) and lambda (λ), based on the amino acid sequences of their constant domains. In short, any such antibody that binds to or associates with human DLL3 is compatible with the teachings herein and may be used as the binding domain component for the disclosed chimeric antigen receptors.

The variable domains of antibodies show considerable variation in amino acid composition from one antibody to another and are primarily responsible for antigen recognition and binding. Variable regions of each light/heavy chain pair form the antibody binding site such that an intact IgG antibody has two binding sites (i.e. it is bivalent). VH and VL domains comprise three regions of extreme variability, which are termed hypervariable regions, or more commonly, complementarity-determining regions (CDRs), framed and separated by four less variable regions known as framework regions (FRs). The non-covalent association between the VH and the VL region forms the Fv fragment (for “fragment variable”) which contains one of the two antigen-binding sites of an intact antibody. Of particular interest scFv constructs (for single chain fragment variable), which can be obtained by genetic engineering as discussed more extensively below, join VH and the VL regions (preferably from the same antibody), though a peptide linker. Depending on the desired conformation it will be appreciated that the peptide linker may be of various lengths.

As used herein, the assignment of amino acids to each domain, framework region and CDR may be in accordance with one of the numbering schemes provided by Kabat et al. (1991) Sequences of Proteins of Immunological Interest (5^{th} Ed.), US Dept. of Health and Human Services, PHS, NIH, NIH Publication no. 91-3242; Chothia et al., 1987, PMID: 3681981; Chothia et al., 1989, PMID: 2687698; MacCallum et al., 1996, PMID: 8876650; or Dubel, Ed. (2007) Handbook of Therapeutic Antibodies, 3^{rd} Ed., Wily-VCH Verlag GmbH and Co or AbM (Oxford Molecular/MSI Pharmacopia) unless otherwise noted. The amino acid residues which comprise CDRs as defined by Kabat, Chothia, MacCallum (also known as Contact) and AbM schemes, as obtained from the Abysis website database (infra.), are set out below.

TABLE 1

| | Kabat | Chothia | MacCallum | AbM |
|---|---|---|---|---|
| VH CDR1 | 31-35 | 26-32 | 30-35 | 26-35 |
| VH CDR2 | 50-65 | 52-56 | 47-58 | 50-58 |
| VH CDR3 | 95-102 | 95-102 | 93-101 | 95-102 |
| VL CDR1 | 24-34 | 24-34 | 30-36 | 24-34 |
| VL CDR2 | 50-56 | 50-56 | 46-55 | 50-56 |
| VL CDR3 | 89-97 | 89-97 | 89-96 | 89-97 |

Variable regions and CDRs in an antibody sequence can be identified according to general rules that have been developed in the art (as set out above, such as, for example, the Kabat numbering system) or by aligning the sequences against a database of known variable regions. Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N.J., 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005). Preferably antibody sequences are analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. Unless otherwise indicated, all CDRs set forth herein are derived according to the Abysis database website as per Kabat et al.

For heavy chain constant region amino acid positions discussed in the invention, numbering is according to the Eu index first described in Edelman et al., 1969, Proc. Natl. Acad. Sci. USA 63(1): 78-85 describing the amino acid sequence of myeloma protein Eu, which reportedly was the first human IgG1 sequenced. The EU index of Edelman is also set forth in Kabat et al., 1991 (supra.). Thus, the terms “EU index as set forth in Kabat” or “EU index of Kabat” or “EU index” in the context of the heavy chain refers to the residue numbering system based on the human IgG1 Eu antibody of Edelman et al. as set forth in Kabat et al., 1991 (supra.) The numbering system used for the light chain constant region amino acid sequence is similarly set forth in Kabat et al., (supra.) An exemplary kappa light chain constant region amino acid sequence compatible with the present invention is set forth immediately below:

(SEQ ID NO: 5)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

Similarly, an exemplary IgG1 heavy chain constant region amino acid sequence compatible with the present invention is set forth immediately below:

(SEQ ID NO: 6)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG.

The disclosed constant region sequences, or variations or derivatives thereof, may be operably associated with the disclosed heavy and light chain variable regions using standard molecular biology techniques to provide antibodies (full length or immunoreactive fragments comprising partial fc regions) that may be used as such or incorporated in DLL3 CARs of the invention (preferably as part of the transmembrane domain).

More generally, the anti-DLL3 binding domain component of compatible CARs may be generated from any antibody that specifically recognizes or associates with a DLL3 determinant. As used herein “determinant” or “target” means any detectable trait, property, marker or factor that is identifiably associated with, or specifically found in or on a particular cell, cell population or tissue. Determinants or targets may be morphological, functional or biochemical in nature and are preferably phenotypic. In certain preferred embodiments a determinant is a protein that is differentially expressed (over- or under-expressed) by specific cell types or by cells under certain conditions (e.g., during specific points of the cell cycle or cells in a particular niche). For the purposes of the instant invention a determinant preferably is differentially expressed on aberrant cancer cells and may comprise a DLL3 protein, or any of its splice variants, isoforms, homologs or family members, or specific domains, regions or epitopes thereof. An “antigen”, “immunogenic determinant”, “antigenic determinant” or “immunogen” means any protein or any fragment, region or domain thereof that can stimulate an immune response when introduced into an immunocompetent animal and is recognized by the antibodies produced from the immune response. The presence or absence of the DLL3 determinants contemplated herein may be used to identify a cell, cell subpopulation or tissue (e.g., tumors, tumorigenic cells or CSCs).

2. Antibody Generation and Production

Antibodies compatible with the invention can be produced using a variety of methods known in the art and any such antibodies may be further modified to provide the binding domain of the anti-DLL3 chimeric antigen receptors of the instant invention.

a. Generation of Polyclonal Antibodies in Host Animals

The production of polyclonal antibodies in various host animals is well known in the art (see for example, Harlow and Lane (Eds.) (1988) Antibodies: A Laboratory Manual, CSH Press; and Harlow et al. (1989) Antibodies, NY, Cold Spring Harbor Press). In order to generate polyclonal antibodies, an immunocompetent animal (e.g., mouse, rat, rabbit, goat, non-human primate, etc.) is immunized with an antigenic protein or cells or preparations comprising an antigenic protein. After a period of time, polyclonal antibody-containing serum is obtained by bleeding or sacrificing the animal. The serum may be used in the form obtained from the animal or the antibodies may be partially or fully purified to provide immunoglobulin fractions or isolated antibody preparations.

Any form of antigen, or cells or preparations containing the antigen, can be used to generate an antibody that is specific for a determinant. The term “antigen” is used in a broad sense and may comprise any immunogenic fragment or determinant of the selected target including a single epitope, multiple epitopes, single or multiple domains or the entire extracellular domain (ECD). The antigen may be an isolated full-length protein, a cell surface protein (e.g., immunizing with cells expressing at least a portion of the antigen on their surface), or a soluble protein (e.g., immunizing with only the ECD portion of the protein). The antigen may be produced in a genetically modified cell. Any of the aforementioned antigens may be used alone or in combination with one or more immunogenicity enhancing adjuvants known in the art. The DNA encoding the antigen may be genomic or non-genomic (e.g., cDNA) and may encode at least a portion of the ECD, sufficient to elicit an immunogenic response. Any vectors may be employed to transform the cells in which the antigen is expressed, including but not limited to adenoviral vectors, lentiviral vectors, plasmids, and non-viral vectors, such as cationic lipids.

b. Monoclonal Antibodies

In selected embodiments, the invention contemplates use of monoclonal antibodies. The term “monoclonal antibody” or “mAb” refers to an antibody obtained from a population of substantially homogeneous antibodies, i.e., the individual antibodies comprising the population are identical except for possible mutations (e.g., naturally occurring mutations), that may be present in minor amounts.

Monoclonal antibodies can be prepared using a wide variety of techniques including hybridoma techniques, recombinant techniques, phage display technologies, transgenic animals (e.g., a XenoMouse®) or some combination thereof. For example, in preferred embodiments monoclonal antibodies can be produced using hybridoma and biochemical and genetic engineering techniques such as described in more detail in An, Zhigiang (ed.) Therapeutic Monoclonal Antibodies: From Bench to Clinic, John Wiley and Sons, 1^{st} ed. 2009; Shire et. al. (eds.) Current Trends in Monoclonal Antibody Development and Manufacturing, Springer Science+Business Media LLC, 1^{st} ed. 2010; Harlow et al., Antibodies: A Laboratory Manual, Cold Spring Harbor Laboratory Press, 2nd ed. 1988; Hammerling, et al., in: Monoclonal Antibodies and T-Cell Hybridomas 563-681 (Elsevier, N.Y., 1981). Following generation of a number of monoclonal antibodies that bind specifically to a determinant, particularly suitable antibodies may be selected through various screening processes, based on, for example, affinity for the determinant or rate of internalization. In particularly preferred embodiments monoclonal antibodies produced as described herein may be used as “source” antibodies and further modified to provide effective DLL3 binding domains that may be associated with the disclosed CARs. For example the source antibody may be manipulated to provide scFvs or other fragments, improve affinity for the target, improve its production in cell culture, reduce immunogenicity in vivo, create multispecific constructs, etc. A more detailed description of monoclonal antibody production and screening is set out below and in the appended Examples.

c. Human Antibodies

Antibodies compatible with the instant invention may comprise fully human antibodies. The term “human antibody” refers to an antibody (preferably a monoclonal antibody) which possesses an amino acid sequence that corresponds to that of an antibody produced by a human and/or has been made using any of the techniques for making human antibodies described below.

In one embodiment, recombinant human antibodies may be isolated by screening a recombinant combinatorial antibody library prepared using phage display. In one embodiment, the library is a scFv phage or yeast display library, generated using human VL and VH cDNAs prepared from mRNA isolated from B-cells.

Human antibodies can also be made by introducing human immunoglobulin loci into transgenic animals, e.g., mice in which the endogenous immunoglobulin genes have been partially or completely inactivated and human immunoglobulin genes have been introduced. Upon challenge antibody generation is observed which closely resembles that seen in humans in all respects, including gene rearrangement, assembly and fully human antibody repertoire. This approach is described, for example, in U.S.P.Ns. 5,545,807; 5,545,806; 5,569,825; 5,625,126; 5,633,425; 5,661,016, and U.S.P.Ns. 6,075,181 and 6,150,584 regarding XenoMouse® technology; and Lonberg and Huszar, 1995, PMID: 7494109). Alternatively, a human antibody may be prepared via immortalization of human B lymphocytes producing an antibody directed against a target antigen (such B lymphocytes may be recovered from an individual suffering from a neoplastic disorder or may have been immunized in vitro). See, e.g., Cole et al., Monoclonal Antibodies and Cancer Therapy, Alan R. Liss, p. 77 (1985); Boerner et al., 1991, PMID: 2051030; and U.S.P.N. 5,750,373. As with other monoclonal antibodies such human antibodies may be used as source antibodies.

d. Antibody Production and Engineering

Antibodies and fragments thereof may be produced or modified using genetic material obtained from antibody producing cells and recombinant technology (see, for example, Berger and Kimmel, Guide to Molecular Cloning Techniques, Methods in Enzymology vol. 152 Academic Press, Inc., San Diego, Calif.; Sambrook and Russell (Eds.) (2000) Molecular Cloning: A Laboratory Manual (3^{rd} Ed.), NY, Cold Spring Harbor Laboratory Press; Ausubel et al. (2002) Short Protocols in Molecular Biology: A Compendium of Methods from Current Protocols in Molecular Biology, Wiley, John & Sons, Inc.; and U.S.P.N. 7,709,611).

As will be discussed in more detail below another aspect of the invention pertains to nucleic acid molecules that encode the DLL3 binding domains and CARs of the invention. The nucleic acids may be present in whole cells, in a cell lysate, or in a partially purified or substantially pure form. A nucleic acid is “isolated” or rendered substantially pure when separated from other cellular components or other contaminants, e.g., other cellular nucleic acids or proteins, by standard techniques, including alkaline/SDS treatment, CsCl banding, column chromatography, agarose gel electrophoresis and others well known in the art. A nucleic acid of the invention can be, for example, DNA (e.g. genomic DNA, cDNA), RNA and artificial variants thereof (e.g., peptide nucleic acids), whether single-stranded or double-stranded or RNA, RNA and may or may not contain introns. In a preferred embodiment, the nucleic acid is a cDNA molecule.

Nucleic acids of the invention can be obtained and manipulated using standard molecular biology techniques. For antibodies expressed by hybridomas (e.g., hybridomas prepared as set forth in the Examples below), cDNAs encoding the light and heavy chains of the antibody can be obtained by standard PCR amplification or cDNA cloning techniques. For antibodies obtained from an immunoglobulin gene library (e.g., using phage display techniques), nucleic acid encoding the antibody can be recovered from the library.

DNA fragments encoding VH and VL segments can be further manipulated by standard recombinant DNA techniques, for example to convert the variable region genes to full-length antibody chain genes, to Fab fragment genes or preferably to a nucleotide sequence encoding a DLL3 specific scFv. In these manipulations, a VL- or VH-encoding DNA fragment is operatively linked to another DNA fragment encoding another protein, such as an antibody constant region or a flexible linker. The term “operatively linked” or “operably linked”, as used in this context, means that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, et al. (1991) (supra)) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. An exemplary IgG1 constant region is set forth in SEQ ID NO: 6. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, et al. (1991) (supra)) and DNA fragments encompassing these regions can be obtained by standard PCR amplification. The light chain constant region can be a kappa or lambda constant region, but most preferably is a kappa constant region. In this respect an exemplary compatible kappa light chain constant region is set forth in SEQ ID NO: 5.

Contemplated herein are certain polypeptides (e.g. antibody variable regions) that exhibit “sequence identity”, sequence similarity” or “sequence homology” to the polypeptides of the invention. A “homologous” polypeptide may exhibit 65%, 70%, 75%, 80%, 85%, or 90% sequence identity. In other embodiments a “homologous” polypeptides may exhibit 93%, 95% or 98% sequence identity. As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

Residue positions which are not identical may differ by conservative amino acid substitutions or by non-conservative amino acid substitutions. A “conservative amino acid substitution” is one in which an amino acid residue is substituted by another amino acid residue having a side chain with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. In cases where there is a substitution with a non-conservative amino acid, in preferred embodiments the polypeptide exhibiting sequence identity will retain the desired function or activity of the polypeptide of the invention (e.g., antibody.)

Also contemplated herein are nucleic acids that that exhibit “sequence identity”, sequence similarity” or “sequence homology” to the nucleic acids of the invention. A “homologous sequence” means a sequence of nucleic acid molecules exhibiting at least about 65%, 70%, 75%, 80%, 85%, or 90% sequence identity. In other embodiments, a “homologous sequence” of nucleic acids may exhibit 93%, 95% or 98% sequence identity to the reference nucleic acid cells or CSCs).

3. Derived Antibodies as DLL3 Binding Domains

Once the source antibodies have been generated, selected and isolated as described above, they may be further altered to provide anti-DLL3 CAR binding domain components compatible with the teachings herein. Preferably the source antibodies are modified or altered using known molecular engineering techniques to provide derived binding domain components having the desired therapeutic properties.

a. Chimeric and Humanized Antibodies

As discussed above selected embodiments of the invention comprise murine monoclonal antibodies that immunospecifically bind to DLL3 and, for the purposes of the instant disclosure, may be considered “source” antibodies for DLL3 binding domains. In selected embodiments, DLL3 binding domains compatible with the invention can be derived from such source antibodies through optional modification of the constant region and/or the antigen binding amino acid sequences of the source antibody. In certain embodiments an antibody is derived from a source antibody if selected amino acids in the source antibody are altered through deletion, mutation, substitution, integration or combination. In another embodiment, a “derived” antibody is one in which fragments of the source antibody (e.g., one or more CDRs or the entire heavy and light chain variable regions) are combined with or incorporated into an acceptor binding domain construct to provide the derivative DLL3 binding domain (e.g. chimeric or humanized binding domains). These derived binding domains can be generated using standard molecular biological techniques as described below, such as, for example, to provide an scFv; to improve affinity for the determinant; to improve antibody stability; to improve expression; to reduce immunogenicity in vivo; to reduce toxicity or to facilitate transmission of a signal. Such antibodies may also be derived from source antibodies through modification of the mature molecule (e.g., glycosylation patterns or pegylation) by chemical means or post-translational modification.

In one embodiment, the chimeric binding regions of the invention are derived from protein segments from at least two different species or class of antibodies that have been covalently joined. The term “chimeric” antibody is directed to constructs in which a portion of the heavy and/or light chain is identical or homologous to corresponding sequences in antibodies from a particular species or belonging to a particular antibody class or subclass, while the remainder of the chain(s) is identical or homologous to corresponding sequences in antibodies from another species or belonging to another antibody class or subclass, as well as fragments of such antibodies (U.S. P.N. 4,816,567; Morrison et al., 1984, PMID: 6436822). In some preferred embodiments chimeric antibodies of the instant invention may comprise all or most of the selected murine heavy and light chain variable regions operably linked to all or part of human light and heavy chain constant regions. In other particularly preferred embodiments, DLL3 binding domains may be “derived” from the mouse antibodies disclosed herein.

In other embodiments, the chimeric binding domains of the invention are “CDR grafted” where the CDRs (as defined using Kabat, Chothia, McCallum, etc.) are derived from a particular species or belonging to a particular antibody class or subclass, while the remainder of the binding region is derived from an antibody from another species or belonging to another antibody class or subclass. For use in humans, one or more selected rodent CDRs (e.g., mouse CDRs) may be grafted into a human acceptor binding domain (i.e., with human framework regions), replacing one or more of the naturally occurring CDRs of the human antibody. These constructs generally have the advantages of providing effective binding while reducing unwanted immune responses to the binding domain by the subject. In particularly preferred embodiments the CDR grafted binding domains will comprise one or more CDRs obtained from a mouse incorporated in a human framework sequence.

Similar to the CDR-grafted binding domain is a “humanized” binding domain. As used herein, a “humanized” binding domain is a human binding domain (acceptor domain generally comprising human framework regions) comprising one or more amino acid sequences (e.g. CDR sequences) derived from one or more non-human antibodies (a donor or source antibody). In certain embodiments, “back mutations” can be introduced into the humanized binding domain, in which residues in one or more FRs of the variable region of the recipient human binding domain are replaced by corresponding residues from the non-human species donor antibody. Such back mutations may to help maintain the appropriate three-dimensional configuration of the grafted CDR(s) and thereby improve affinity and binding domain stability. Antibodies from various donor species may be used including, without limitation, mouse, rat, rabbit, or non-human primate. Furthermore, humanized antibodies or fragments may comprise new residues that are not found in the recipient antibody or in the donor antibody to, for example, further refine antibody performance. CDR grafted and humanized antibodies (and related DLL3 binding domains) compatible with the instant invention and comprising the source murine antibodies set forth in the Examples below may therefor readily be provided without undue experimentation using the prior art techniques as set forth herein.

Various art-recognized techniques can further be used to determine which human sequences to use as acceptor antibodies to provide humanized constructs in accordance with the instant invention. Compilations of compatible human germline sequences and methods of determining their suitability as acceptor sequences are disclosed, for example, in Tomlinson, I. A. et al. (1992) J. Mol. Biol. 227:776-798; Cook, G. P. et al. (1995) Immunol. Today 16: 237-242; Chothia, D. et al. (1992) J. Mol. Biol. 227:799-817; and Tomlinson et al. (1995) EMBO J 14:4628-4638 each of which is incorporated herein in its entirety. The V-BASE directory (VBASE2—Retter et al., Nucleic Acid Res. 33; 671-674, 2005) which provides a comprehensive directory of human immunoglobulin variable region sequences (compiled by Tomlinson, I. A. et al. MRC Centre for Protein Engineering, Cambridge, UK) may also be used to identify compatible acceptor sequences. Additionally, consensus human framework sequences described, for example, in U.S.P.N. 6,300,064 may also prove to be compatible acceptor sequences are can be used in accordance with the instant teachings. In general, human framework acceptor sequences are selected based on homology with the murine source framework sequences along with an analysis of the CDR canonical structures of the source and acceptor antibodies. The derived sequences of the heavy and light chain variable regions of the derived antibody (or binding domain) may then be synthesized using art recognized techniques.

By way of example CDR grafted and humanized antibodies, and associated methods, are described in U.S.P.Ns. 6,180,370 and 5,693,762. For further details, see, e.g., Jones et al., 1986, PMID: 3713831); and U.S.P.Ns. 6,982,321 and 7,087,409.

The sequence identity or homology of the CDR grafted or humanized antibody variable region to the human acceptor variable region may be determined as discussed herein and, when measured as such, will preferably share at least 60% or 65% sequence identity, more preferably at least 70%, 75%, 80%, 85%, or 90% sequence identity, even more preferably at least 93%, 95%, 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution.

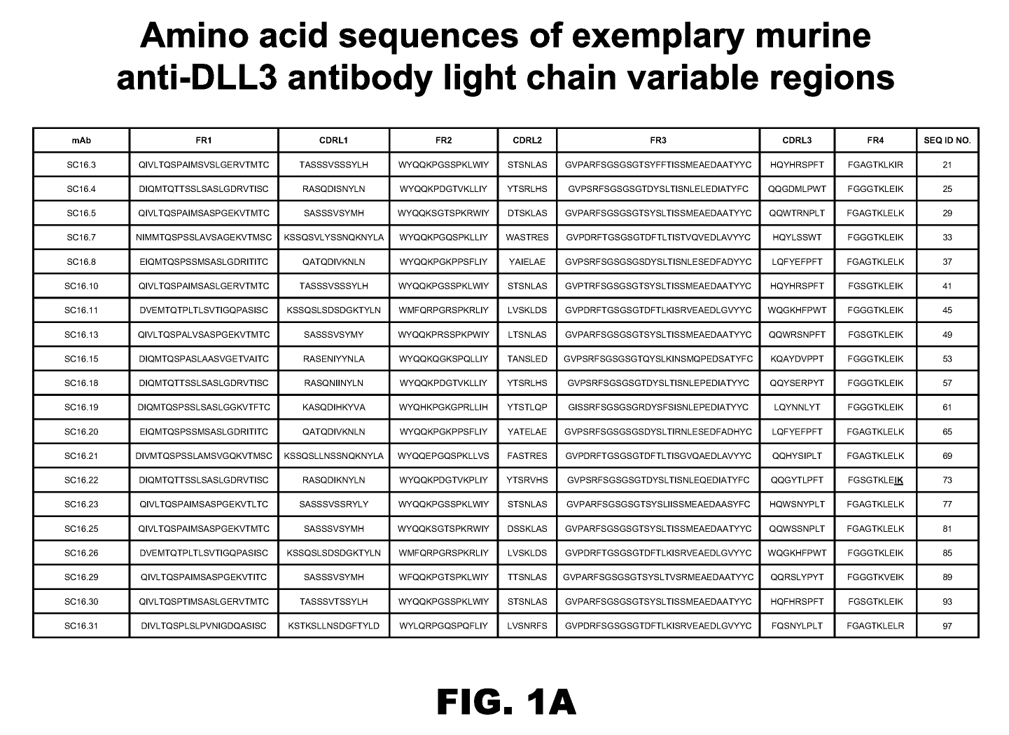

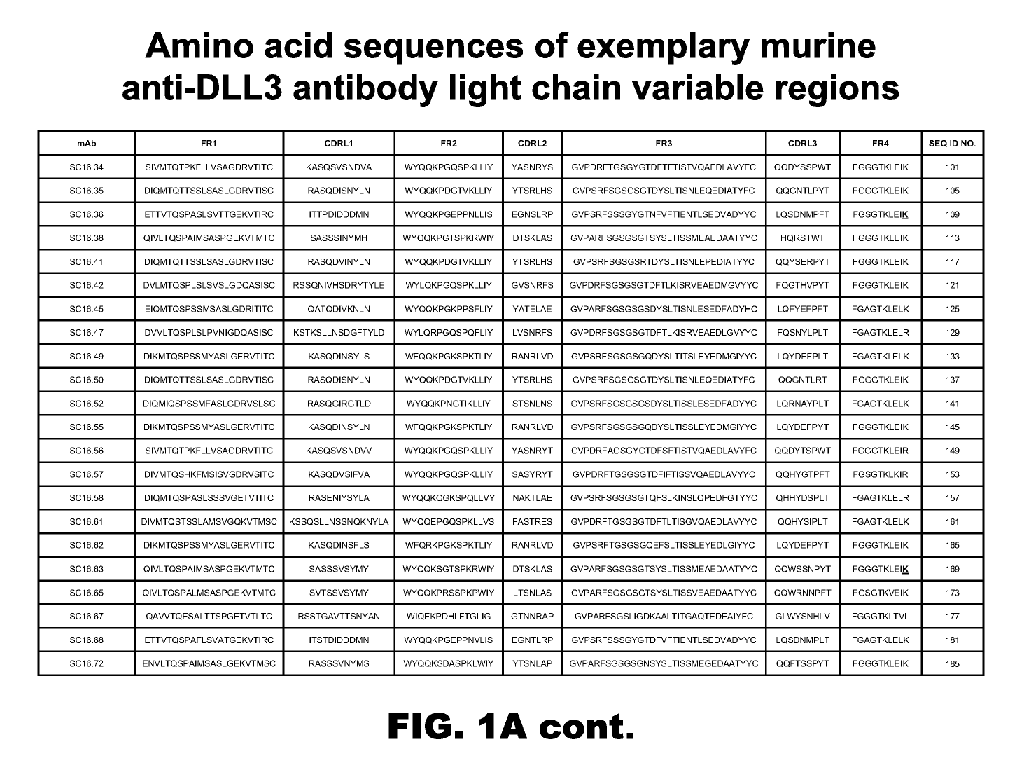

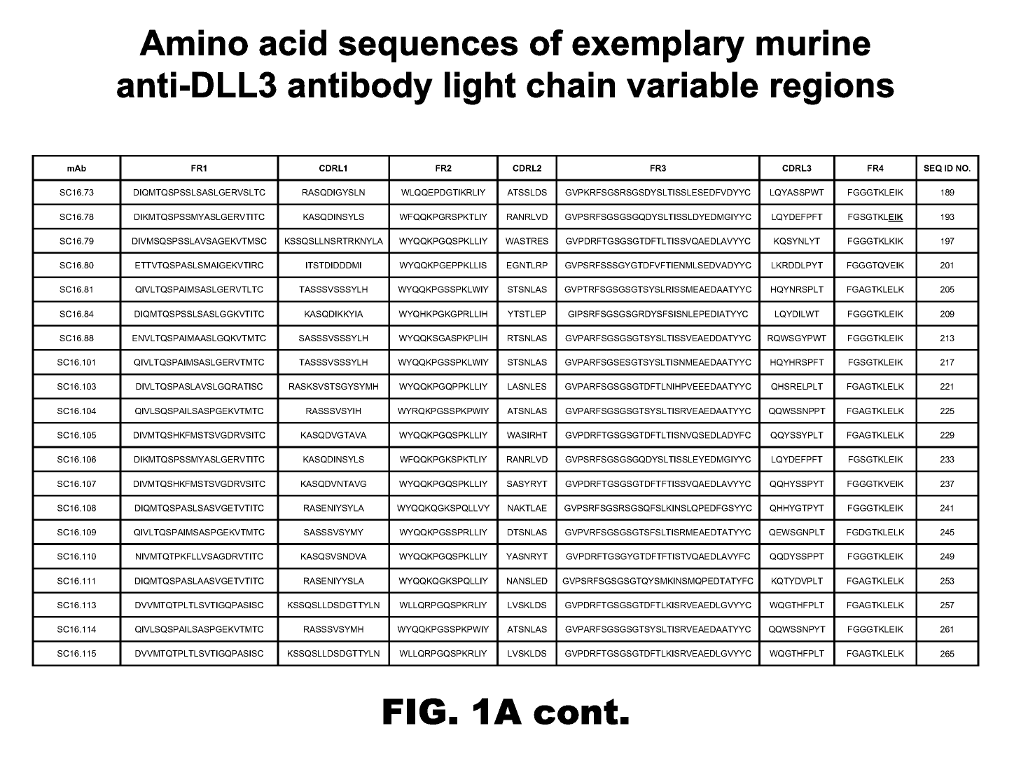

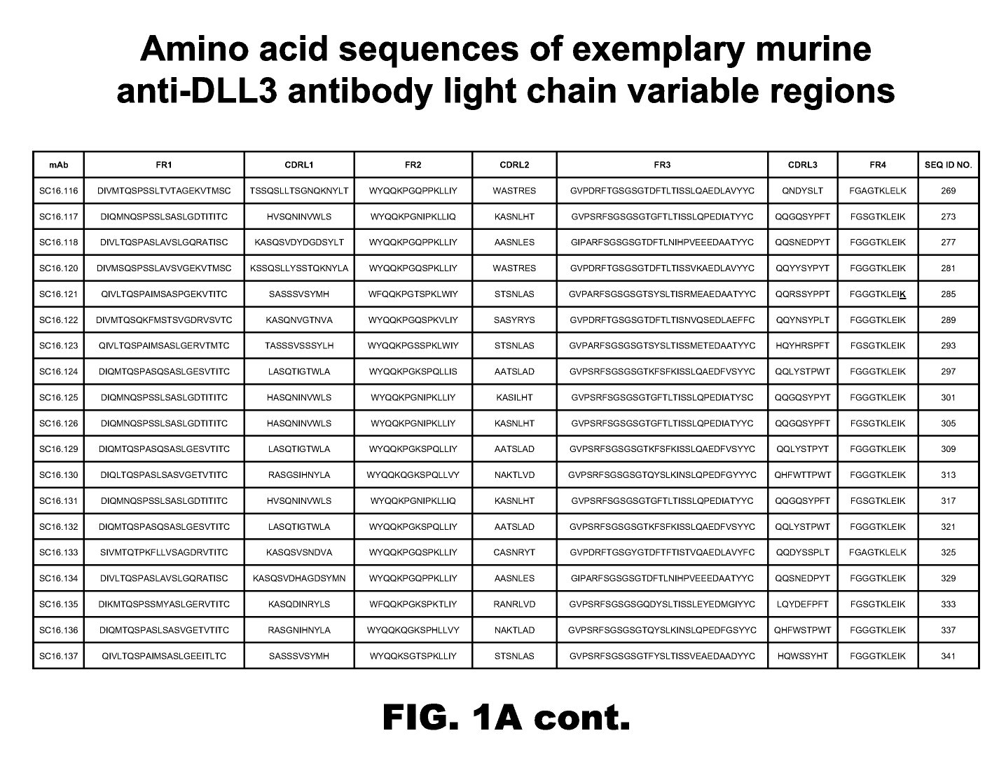

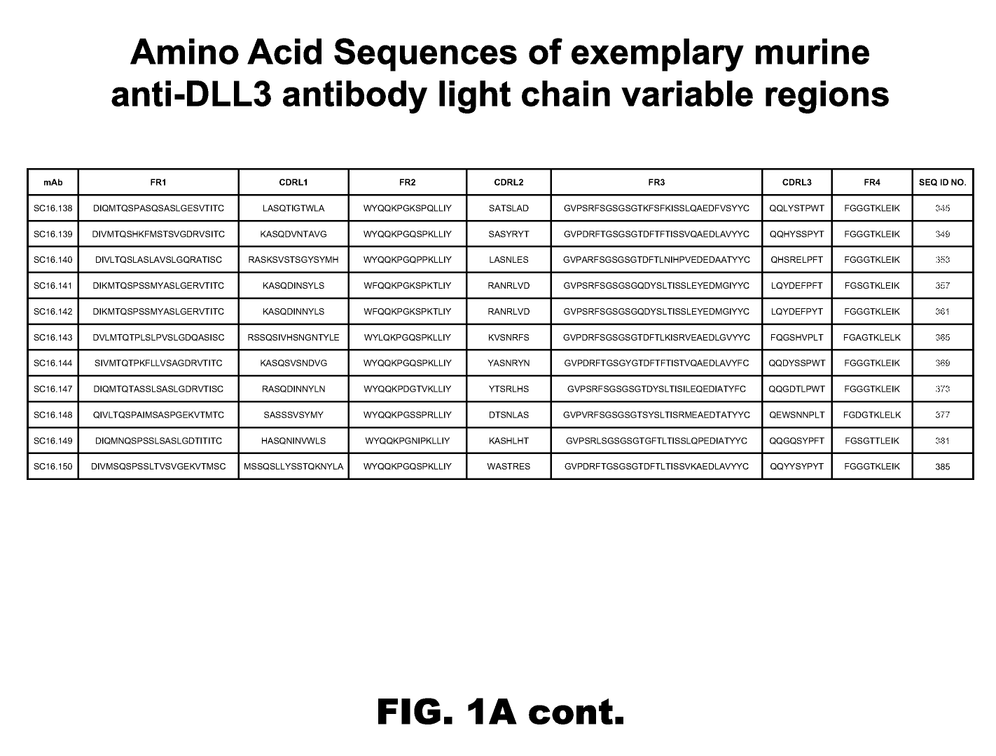

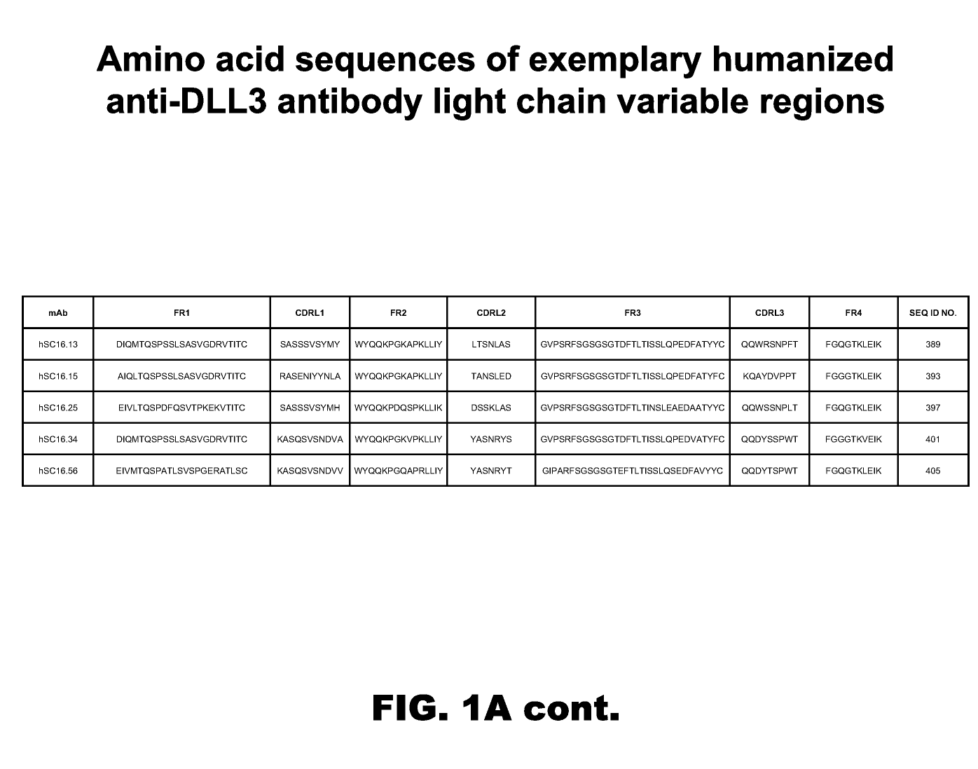

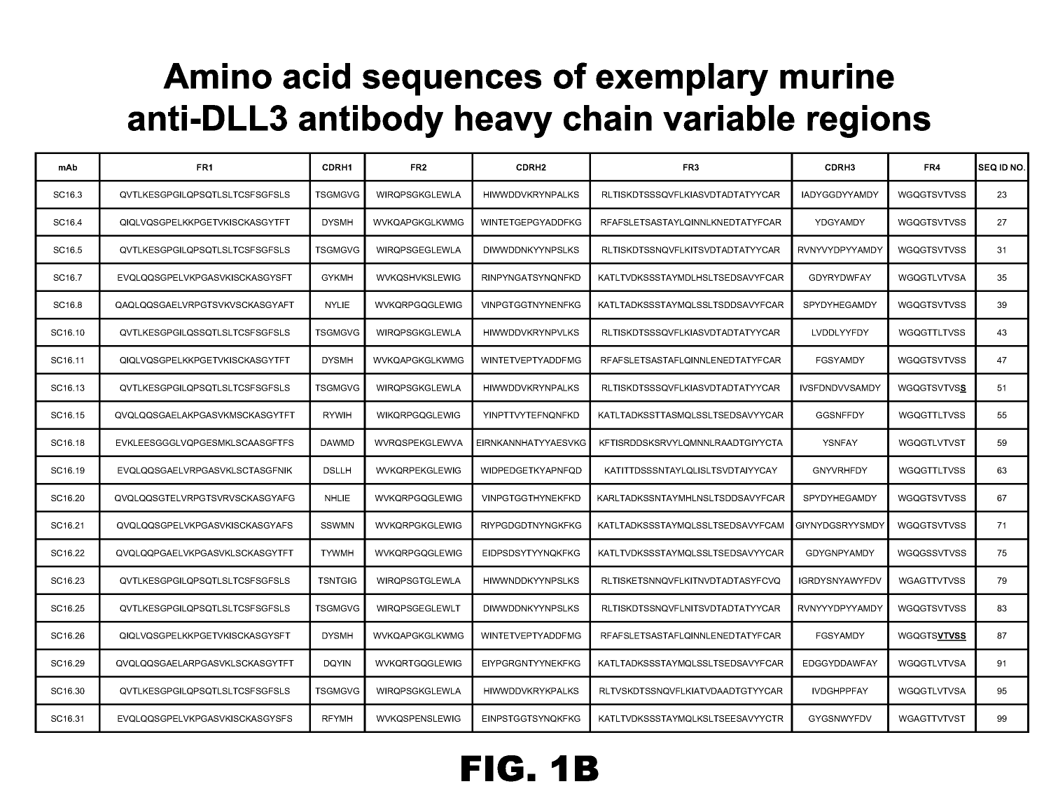

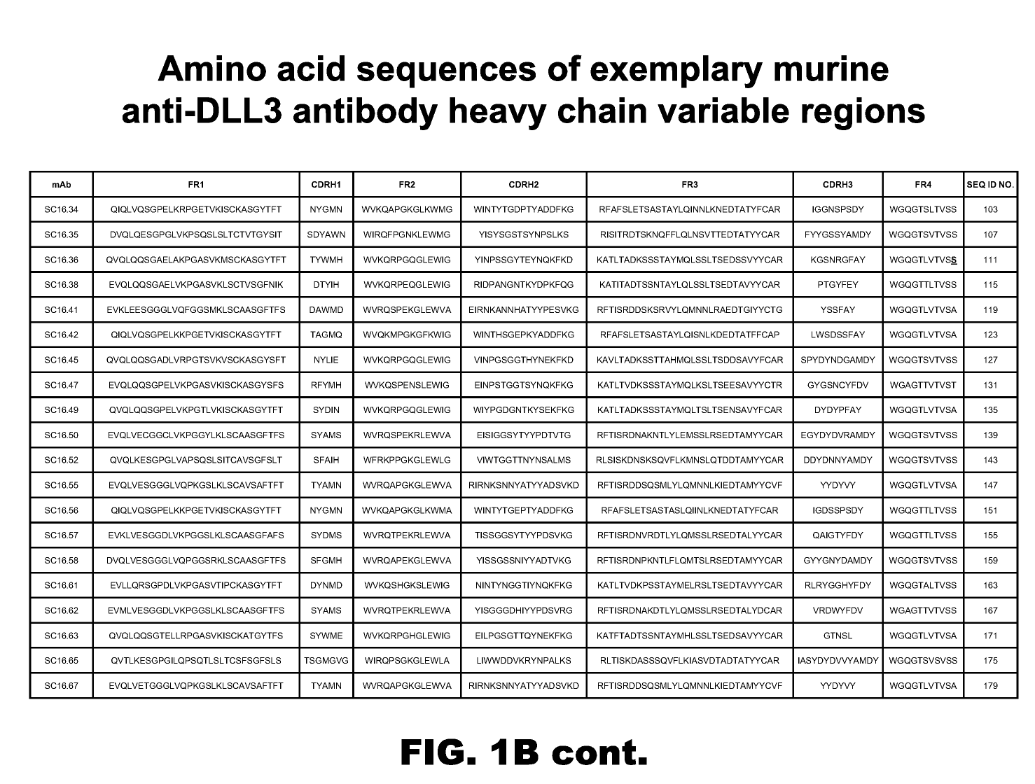

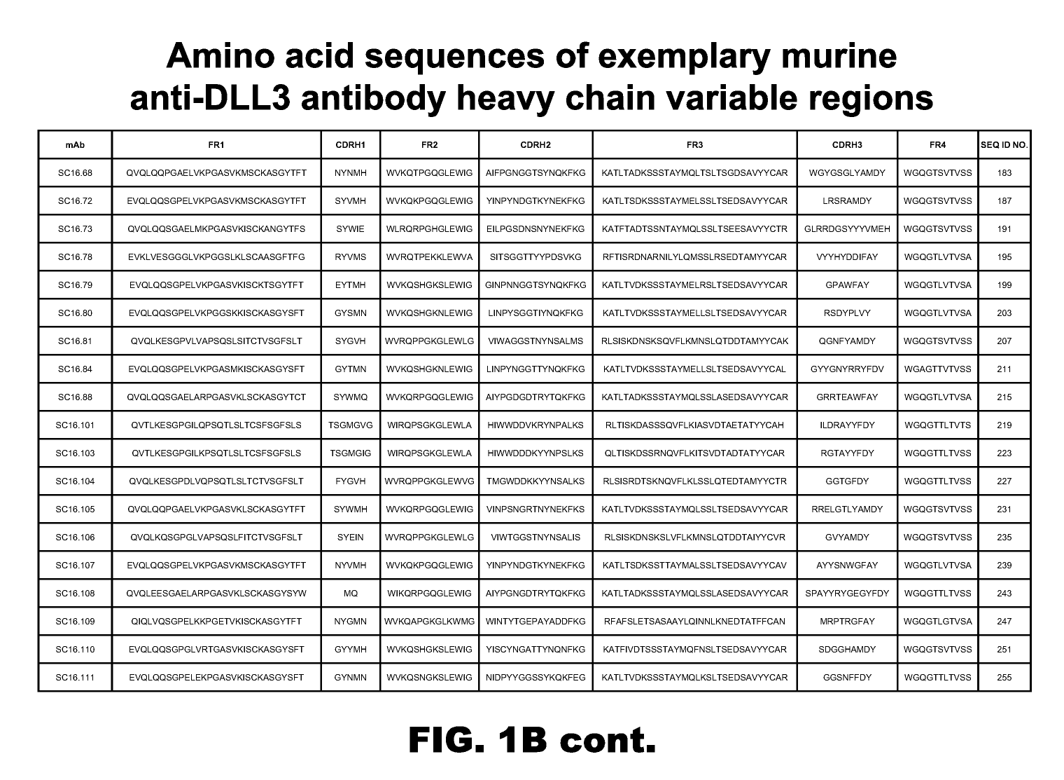

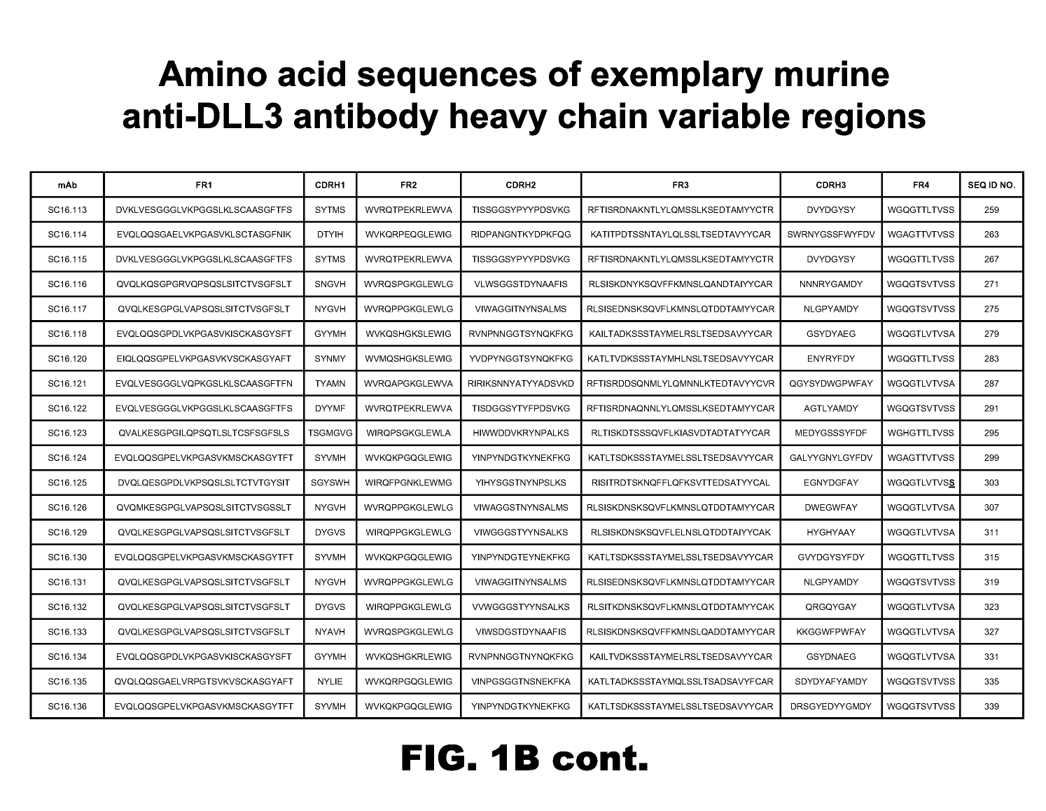

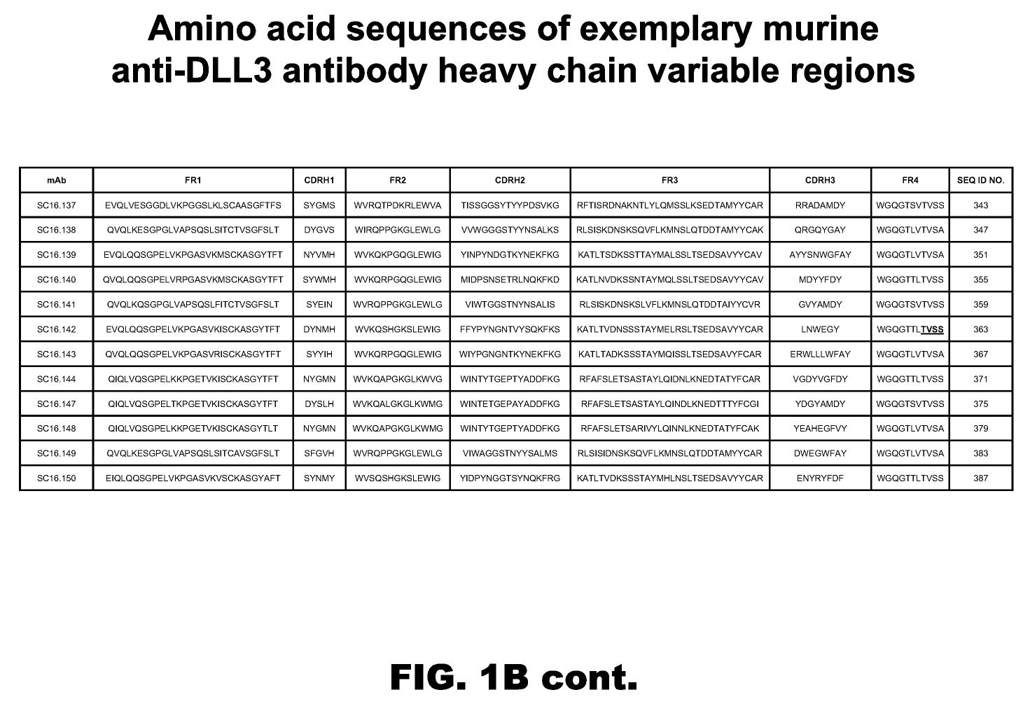

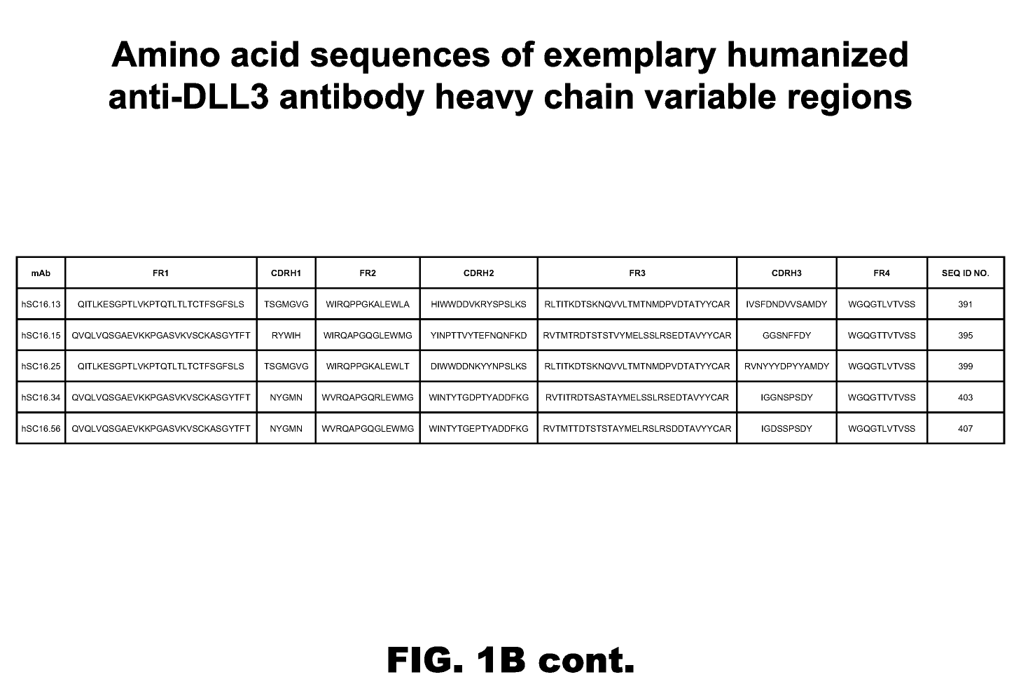

It will be appreciated that the annotated CDRs and framework sequences as provided in the appended FIGS. 1A and 1B are defined as per Kabat et al. using a proprietary Abysis database. However, as discussed herein one skilled in the art could readily identify CDRs in accordance with definitions provided by Chothia et al., ABM or MacCallum et al as well as Kabat et al. As such, anti-DLL3 humanized antibodies comprising one or more CDRs derived according to any of the aforementioned systems are explicitly held to be within the scope of the instant invention.

b. Antibody Fragments, Derivatives or Constructs

In particularly preferred embodiments the DLL3 binding domain will comprise an antibody fragment, derivative or construct. More particularly, regardless of which form of antibody (e.g. chimeric, humanized, etc.) is selected to practice the invention it will be appreciated that immunoreactive fragments of the same may be used, as part of a DLL3 CAR, in accordance with the teachings herein. In a broad sense an “antibody fragment” comprises at least an immunoreactive portion of an intact antibody. That is, as used herein, the term “antibody fragment” includes at least an antigen-binding fragment or portion of an intact antibody and the term “antigen-binding fragment” refers to a polypeptide fragment of an immunoglobulin or antibody that immunospecifically binds or reacts with an immunogenic determinant of DLL3 or competes with the intact antibody from which the fragments were derived for specific antigen binding. Moreover, for the purposes of the instant invention an “antibody construct” or “antibody derivative” shall be held to mean any molecular structure comprising an antibody fragment. Preferably such derivatives or constructs shall be non-natural and will be fabricated to impart beneficial molecular properties while maintaining the immunoreactive (or immunospecific) nature of the antibody.

Exemplary compatible antibody fragments, constructs or derivatives include: variable light chain fragments (VL), variable heavy chain fragments (VH), scFv, F(ab′)2 fragment, Fab fragment, Fd fragment, Fv fragment, single domain antibody fragments, diabodies, linear antibodies, single-chain antibody molecules and multispecific antibodies formed or derived from antibody fragments. In other embodiments the DLL3 binding domain of the instant invention may comprise an intact antibody, a scFv-Fc construct, a minibody, a diabody, a scFv construct, a Fab-scFv2 construct, a Fab-scFv construct or a peptibody. In certain aspects the DLL3 binding domain will be covalently linked (e.g., by using art-recognized genetic engineering techniques) to the transmembrane and intracellular domains of the CAR. In other embodiments the DLL3 binding domain may be non-covalently linked (e.g., via an Fc portion of the binding domain as set forth in U.S.P.N. 2015/0139943) to the transmembrane and intracellular domains of the CAR. Each form of binding domain attachment is compatible with the instant invention as long as the sensitized lymphocytes are able to induce the desired immune response.

In particularly preferred embodiments, and as shown in the appended Examples, the DLL3 binding domain will comprise a scFv construct. As used herein, a “single chain variable fragment (scFv)” means a single chain polypeptide derived from an antibody which retains the ability to bind to an antigen. An example of the scFv includes an antibody polypeptide which is formed by a recombinant DNA technique and in which Fv regions of immunoglobulin heavy chain and light chain fragments are linked via a spacer sequence. Various methods for preparing an scFv are known, and include methods described in U.S.P.N. 4,694,778.

In other embodiments, the DLL3 binding domain is one that comprises an Fc region and that retains at least one of the biological functions normally associated with the Fc region when present in an intact antibody, such as FcRn binding, antibody half-life modulation, ADCC function and complement binding. In one embodiment, an antibody fragment is a monovalent antibody that has an in vivo half-life substantially similar to an intact antibody. For example, such a binding domain may comprise an immunoreactive region linked to an Fc sequence comprising at least one free cysteine capable of conferring in vivo stability to the fragment. In other embodiments the Fc region may be modified using art-recognized techniques to modify the pharmacokinetics or pharmacodynamics of the disclosed CARs and sensitized lymphocytes.

Where the DLL3 binding domain comprises an Fc portion it may be non-covalently linked or joined with the remaining portions of the CAR via an extracellular Fc receptor or binding molecule (“Fc binder”) that is operably associated with the transmembrane and intracellular domains. As used herein the term “Fc binder” is held to mean any molecule or portion thereof that binds to, or associates with, the Fc portion of an antibody (e.g., an Fc receptor). Such constructs (i.e., a “proto-CAR” comprising an Fc binder, transmembrane domain and intracellular signaling domain) may be fabricated using standard molecular biology techniques and associated with the selected lymphocytes (autologous or allogeneic) as described herein (e.g., via transduction) to generate “primed lymphocytes”. At some point prior to introduction into the patient, the primed lymphocytes may then be exposed to selected DLL3 binding domain(s) comprising at least an Fc portion under conditions that allow association of the DLL3 binding domain(s) with the proto-CAR. The non-covalent association of the binding domain with the proto-CAR provide the DLL3 sensitized lymphocytes of the instant invention and may be used to inhibit tumorigenic cell proliferation as described herein (see generally U.S.P.N. 2015/0139943 which is incorporated herein in its entirety).

In those embodiments comprising a proto-CAR the Fc binder may comprise an Fc receptor such as an Fc-gamma receptor, an Fc-alpha receptor or an Fc-epsilon receptor. In certain selected embodiments the Fc receptor may comprise the ligand binding domain of CD16 (e.g., CD16A or CD16B), CD32 (e.g., CD32A or CD32B) or CD64 (e.g., CD64A, CD64B or CD64C). In certain other embodiments the Fc binder will not be an Fc receptor. For example the Fc binder may comprise all or part of protein A or protein G as long as the proto-CAR has the ability to associate with the DLL3 binding domain. In other embodiments the Fc binder may comprise an immunoreactive antibody or fragment or construct or derivative thereof that binds the Fc portion of an immunoglobulin. As to such embodiments the Fc binder may, for example comprise an scFv, a nanobody or a minibody. Similarly DLL3 binding domains compatible with such embodiments include any molecule that is capable of being bound by the Fc binder and immunospecifically reacting with DLL3. In some embodiments the DLL3 binding domain will comprise intact DLL3 monoclonal antibodies or mixtures of intact DLL3 monoclonal antibodies. In other embodiments the DLL3 binding domain may comprise intact polyclonal DLL3 antibodies (preferably fully human). In yet other embodiments the DLL3 binding domain may comprise a scFv-Fc construct. More generally, those of skill in the art will readily be able to identify proto-CAR compatible DLL3 binding regions based on the teachings of the instant disclosure.

Moreover, as would be readily recognized by those skilled in the art, the disclosed fragments, construct or derivatives can be obtained by molecular engineering or via chemical or enzymatic treatment (such as papain or pepsin) of an intact or complete antibody or antibody chain or by recombinant means. See, e.g., Fundamental Immunology, W. E. Paul, ed., Raven Press, N.Y. (1999), for a more detailed description of antibody fragments.

c. Post-Production Selection

No matter how obtained, antibody-producing cells (e.g., hybridomas, yeast colonies, etc.) may be selected, cloned and further screened for desirable characteristics including, for example, high affinity for DLL3. Hybridomas can be expanded in vitro in cell culture or in vivo in syngeneic immunocompromised animals. Methods of selecting, cloning and expanding hybridomas and/or colonies are well known to those of ordinary skill in the art. Once the desired antibodies are identified the relevant genetic material may be isolated, manipulated and expressed using common, art-recognized molecular biology and biochemical techniques.

The antibodies produced by naïve libraries (either natural or synthetic) may be of moderate affinity (Kₐ of about 10⁶ to 10⁷ M⁻¹). To enhance affinity, affinity maturation may be mimicked in vitro by constructing antibody libraries (e.g., by introducing random mutations in vitro by using error-prone polymerase) and reselecting antibodies with high affinity for the antigen from those secondary libraries (e.g. by using phage or yeast display). WO 9607754 describes a method for inducing mutagenesis in a CDR of an immunoglobulin light chain to create a library of light chain genes.

Various techniques can be used to select antibodies, including but not limited to, phage or yeast display in which a library of human combinatorial antibodies or scFv fragments is synthesized on phages or yeast, the library is screened with the antigen of interest or an antibody-binding portion thereof, and the phage or yeast that binds the antigen is isolated, from which one may obtain the antibodies or immunoreactive fragments (Vaughan et al., 1996, PMID: 9630891; Sheets et al., 1998, PMID: 9600934; Boder et al., 1997, PMID: 9181578; Pepper et al., 2008, PMID: 18336206). Kits for generating phage or yeast display libraries are commercially available. There also are other methods and reagents that can be used in generating and screening antibody display libraries (see U.S.P.N. 5,223,409; WO 92/18619, WO 91/17271, WO 92/20791, WO 92/15679, WO 93/01288, WO 92/01047, WO 92/09690; and Barbas et al., 1991, PMID: 1896445). Such techniques advantageously allow for the screening of large numbers of candidate antibodies and provide for relatively easy manipulation of sequences (e.g., by recombinant shuffling).

4. Characteristics of DLL3 Binding Domains

In selected embodiments, antibody-producing cells (e.g., hybridomas or yeast colonies) may be selected, cloned and further screened for favorable properties including, for example, robust growth, high antibody production and, as discussed in more detail below, desirable binding domain characteristics. In other cases characteristics of the antibody may be imparted by selecting a particular antigen (e.g., a specific DLL3 domain) or immunoreactive fragment of the target antigen for inoculation of the animal. In still other embodiments the selected antibodies may be engineered as described above to enhance or refine immunochemical characteristics such as affinity or pharmacokinetics fragments.

a. Binding Domain Affinity

Disclosed herein are antibodies that have a high binding affinity for a specific determinant e.g. DLL3. The terms “immunospecifically binds” “specific binding,” “selective binding,” “selectively binds,” and “specifically binds,” refer to antibody binding to an epitope on a predetermined antigen. Typically, the antibody binds with an affinity (K_{D}) of approximately less than 10⁻⁷ M, such as approximately less than 10⁻⁸ M, 10⁻⁹ M or 10⁻¹⁰ M or even lower. The term “K_{D}” refers to the dissociation constant or apparent affinity of a particular antibody-antigen interaction.

More specifically an antibody of the invention can immunospecifically bind its target antigen when the dissociation constant K_{D} (k_{off}/kₒₙ) is ≦10⁻⁷ M. The antibody specifically binds antigen with high affinity when the K_{D} is ≦5×10⁻⁹ M, and with very high affinity when the K_{D} is ≦5×10⁻¹⁰ M. In one embodiment of the invention, the antibody has a K_{D} of ≦10⁻⁹ M and an off-rate of about 1×10⁻⁴/sec. In one embodiment of the invention, the off-rate is <1×10⁻⁵/sec. In other embodiments of the invention, the antibodies will bind to a determinant with a K_{D} of between about 10⁻⁷ M and 10⁻¹⁰ M, and in yet another embodiment it will bind with a K_{D}≦2×10⁻¹⁰ M. Still other selected embodiments of the invention comprise antibodies that have a K_{D} (k_{off}/kₒₙ) of less than 10⁻⁶ M, less than 5×10⁻⁶ M, less than 10⁻⁷ M, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴ M, less than 5×10⁻¹⁴ M, less than 10⁻¹⁵ M or less than 5×10⁻¹⁵ M.

In certain embodiments, an antibody of the invention that immunospecifically binds to a determinant e.g. DLL3 may have an association rate constant or kₒₙ (or kₐ) rate (antibody+antigen (Ag)^{k}ₒₙ←antibody-Ag) of at least 10⁵ M^{-l}s^{-l}, at least 2×10⁵ M^{-l}s^{-l}, at least 5×10⁵ M^{-l}s^{-l}, at least 10⁶ M^{-l}s^{-l}, at least 5×10⁶ M^{-l}s^{-l}, at least 10⁷ M^{-l}s^{-l}, at least 5×10⁷ M^{-l}s^{-l}, or at least 10⁸ M^{-l}s^{-l}.

In another embodiment, an antibody of the invention that immunospecifically binds to a determinant e.g. DLL3 may have a disassociation rate constant or k_{off} (or k_{d}) rate (antibody+antigen (Ag)^{k}_{off}←antibody-Ag) of less than 10^{-l}s^{-l}, less than 5×10^{-l}s^{-l}, less than 10⁻² s^{-l}, less than 5×10⁻² s^{-l}, less than 10⁻³ s^{-l}, less than 5×10⁻³ s^{-l}, less than 10⁻⁴ s^{-l}, less than 5×10⁴ s^{-l}, less than 10⁻⁵ s^{-l}, less than 5×10⁻⁵ s^{-l}, less than 10⁻⁶ s^{-l}, less than 5×10⁻⁶ s^{-l} less than 10⁻⁷ s^{-l}, less than 5×10⁻⁷ s^{-l}, less than 10⁻⁸ s^{-l}, less than 5×10⁻⁸ s^{-l}, less than 10⁻⁹ s^{-l}, less than 5×10⁻⁹ s^{-l} or less than 10⁻¹⁰s^{-l}.

Binding affinity may be determined using various techniques known in the art, for example, surface plasmon resonance, bio-layer interferometry, dual polarization interferometry, static light scattering, dynamic light scattering, isothermal titration calorimetry, ELISA, analytical ultracentrifugation, and flow cytometry.

b. Binning and Epitope Mapping

As used herein, the term “binning” refers to methods used to group antibodies (or binding domains) into “bins” based on their antigen binding characteristics and whether they compete with each other. The initial determination of bins may be further refined and confirmed by epitope mapping and other techniques as described herein. However it will be appreciated that empirical assignment of antibodies to individual bins provides information that may be indicative of the therapeutic potential of the disclosed antibodies.

More specifically, one can determine whether a selected reference antibody (or fragment thereof) competes for binding with a second test antibody (i.e., is in the same bin) by using methods known in the art and set forth in the Examples herein. In one embodiment, a reference antibody is associated with DLL3 antigen under saturating conditions and then the ability of a secondary or test antibody to bind to DLL3 is determined using standard immunochemical techniques. If the test antibody is able to substantially bind to DLL3 at the same time as the reference anti-DLL3 antibody, then the secondary or test antibody binds to a different epitope than the primary or reference antibody. However, if the test antibody is not able to substantially bind to DLL3 at the same time, then the test antibody binds to the same epitope, an overlapping epitope, or an epitope that is in close proximity (at least sterically) to the epitope bound by the primary antibody. That is, the test antibody competes for antigen binding and is in the same bin as the reference antibody.

The term “compete” or “competing antibody” when used in the context of the disclosed antibodies means competition between antibodies as determined by an assay in which a test antibody or immunologically functional fragment being tested inhibits specific binding of a reference antibody to a common antigen. Typically, such an assay involves the use of purified antigen (e.g., DLL3 or a domain or fragment thereof) bound to a solid surface or cells, an unlabeled test antibody and a labeled reference antibody. Competitive inhibition is measured by determining the amount of label bound to the solid surface or cells in the presence of the test antibody. Usually the test antibody is present in excess and/or allowed to bind first. Additional details regarding methods for determining competitive binding are provided in the Examples herein. Usually, when a competing antibody is present in excess, it will inhibit specific binding of a reference antibody to a common antigen by at least 30%, 40%, 45%, 50%, 55%, 60%, 65%, 70% or 75%. In some instance, binding is inhibited by at least 80%, 85%, 90%, 95%, or 97% or more.

Conversely, when the reference antibody is bound it will preferably inhibit binding of a subsequently added test antibody (i.e., a DLL3 antibody) by at least 30%, 40%, 45%, 50%, 55%, 60%, 65%, 70% or 75%. In some instance, binding of the test antibody is inhibited by at least 80%, 85%, 90%, 95%, or 97% or more.

Generally binning or competitive binding may be determined using various art-recognized techniques, such as, for example, immunoassays such as western blots, radioimmunoassays, enzyme linked immunosorbent assay (ELISA), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such immunoassays are routine and well known in the art (see, Ausubel et al, eds, (1994) Current Protocols in Molecular Biology, Vol. 1, John Wiley & Sons, Inc., New York). Additionally, cross-blocking assays may be used (see, for example, WO 2003/48731; and Harlow et al. (1988) Antibodies, A Laboratory Manual, Cold Spring Harbor Laboratory, Ed Harlow and David Lane).

Other technologies used to determine competitive inhibition (and hence “bins”), include: surface plasmon resonance using, for example, the BIAcore™ 2000 system (GE Healthcare); bio-layer interferometry using, for example, a ForteBio® Octet RED (ForteBio); or flow cytometry bead arrays using, for example, a FACSCanto II (BD Biosciences) or a multiplex LUMINEX™ detection assay (Luminex).

Luminex is a bead-based immunoassay platform that enables large scale multiplexed antibody pairing. The assay compares the simultaneous binding patterns of antibody pairs to the target antigen. One antibody of the pair (capture mAb) is bound to Luminex beads, wherein each capture mAb is bound to a bead of a different color. The other antibody (detector mAb) is bound to a fluorescent signal (e.g. phycoerythrin (PE)). The assay analyzes the simultaneous binding (pairing) of antibodies to an antigen and groups together antibodies with similar pairing profiles. Similar profiles of a detector mAb and a capture mAb indicates that the two antibodies bind to the same or closely related epitopes. In one embodiment, pairing profiles can be determined using Pearson correlation coefficients to identify the antibodies which most closely correlate to any particular antibody on the panel of antibodies that are tested. In preferred embodiments a test/detector mAb will be determined to be in the same bin as a reference/capture mAb if the Pearson's correlation coefficient of the antibody pair is at least 0.9. In other embodiments the Pearson's correlation coefficient is at least 0.8, 0.85, 0.87 or 0.89. In further embodiments, the Pearson's correlation coefficient is at least 0.91, 0.92, 0.93, 0.94, 0.95, 0.96, 0.97, 0.98, 0.99 or 1. Other methods of analyzing the data obtained from the Luminex assay are described in U.S.P.N. 8,568,992. The ability of Luminex to analyze 100 different types of beads (or more) simultaneously provides almost unlimited antigen and/or antibody surfaces, resulting in improved throughput and resolution in antibody epitope profiling over a biosensor assay (Miller, et al., 2011, PMID: 21223970).

“Surface plasmon resonance,” refers to an optical phenomenon that allows for the analysis of real-time specific interactions by detection of alterations in protein concentrations within a biosensor matrix.

In other embodiments, a technique that can be used to determine whether a test antibody “competes” for binding with a reference antibody is “bio-layer interferometry”, an optical analytical technique that analyzes the interference pattern of white light reflected from two surfaces: a layer of immobilized protein on a biosensor tip, and an internal reference layer. Any change in the number of molecules bound to the biosensor tip causes a shift in the interference pattern that can be measured in real-time. Such biolayer interferometry assays may be conducted using a ForteBio® Octet RED machine as follows. A reference antibody (Ab1) is captured onto an anti-mouse capture chip, a high concentration of non-binding antibody is then used to block the chip and a baseline is collected. Monomeric, recombinant target protein is then captured by the specific antibody (Ab1) and the tip is dipped into a well with either the same antibody (Ab1) as a control or into a well with a different test antibody (Ab2). If no further binding occurs, as determined by comparing binding levels with the control Ab1, then Ab1 and Ab2 are determined to be “competing” antibodies. If additional binding is observed with Ab2, then Ab1 and Ab2 are determined not to compete with each other. This process can be expanded to screen large libraries of unique antibodies using a full row of antibodies in a 96-well plate representing unique bins. In preferred embodiments a test antibody will compete with a reference antibody if the reference antibody inhibits specific binding of the test antibody to a common antigen by at least 40%, 45%, 50%, 55%, 60%, 65%, 70% or 75%. In other embodiments, binding is inhibited by at least 80%, 85%, 90%0, 95%, or 97% or more.

Once a bin, encompassing a group of competing antibodies, has been defined further characterization can be carried out to determine the specific domain or epitope on the antigen to which the antibodies in a bin bind. Domain-level epitope mapping may be performed using a modification of the protocol described by Cochran et al., 2004, PMID: 15099763. Fine epitope mapping is the process of determining the specific amino acids on the antigen that comprise the epitope of a determinant to which the antibody binds. The term “epitope” is used in its common biochemical sense and refers to that portion of the target antigen capable of being recognized and specifically bound by a particular antibody. In certain embodiments, epitopes or immunogenic determinants include chemically active surface groupings of molecules such as amino acids, sugar side chains, phosphoryl groups, or sulfonyl groups, and, in certain embodiments, may have specific three-dimensional structural characteristics, and/or specific charge characteristics. In certain embodiments, an antibody is said to specifically bind an antigen when it preferentially recognizes its target antigen in a complex mixture of proteins and/or macromolecules.

When the antigen is a polypeptide such as DLL3, epitopes may generally be formed from both contiguous amino acids and noncontiguous amino acids juxtaposed by tertiary folding of a protein (“conformational epitopes”). In such conformational epitopes the points of interaction occur across amino acid residues on the protein that are linearly separated from one another. Epitopes formed from contiguous amino acids (sometimes referred to as “linear” or “continuous” epitopes) are typically retained upon protein denaturing, whereas epitopes formed by tertiary folding are typically lost upon protein denaturing. An antibody epitope typically includes at least 3, and more usually, at least 5 or 8-10 amino acids in a unique spatial conformation. Methods of epitope determination or “epitope mapping” are well known in the art and may be used in conjunction with the instant disclosure to identify epitopes on DLL3 bound by the disclosed antibodies.

Compatible epitope mapping techniques include alanine scanning mutants, peptide blots (Reineke (2004) Methods Mol Biol 248:443-63), or peptide cleavage analysis. In addition, methods such as epitope excision, epitope extraction and chemical modification of antigens can be employed (Tomer (2000) Protein Science 9: 487-496). Other compatible methods comprise yeast display methods. In other embodiments Modification-Assisted Profiling (MAP), also known as Antigen Structure-based Antibody Profiling (ASAP) provides a method that categorizes large numbers of monoclonal antibodies directed against the same antigen according to the similarities of the binding profile of each antibody to chemically or enzymatically modified antigen surfaces (U.S.P.N. 2004/0101920). This technology allows rapid filtering of genetically identical antibodies, such that characterization can be focused on genetically distinct antibodies. It will be appreciated that MAP may be used to sort the DLL3 antibodies of the invention into groups of antibodies binding different epitopes.

Once a desired epitope on an antigen is determined, it is possible to generate antibodies to that epitope, e.g., by immunizing with a peptide comprising the epitope using techniques described in the present invention. Alternatively, during the discovery process, the generation and characterization of antibodies may elucidate information about desirable epitopes located in specific domains or motifs. From this information, it is then possible to competitively screen antibodies for binding to the same epitope. An approach to achieve this is to conduct competition studies to find antibodies that compete for binding to the antigen. A high throughput process for binning antibodies based upon their cross-competition is described in WO 03/48731. Other methods of binning or domain level or epitope mapping comprising antibody competition or antigen fragment expression on yeast are well known in the art.

B. Optional Hinge Region

As used herein, the term “hinge region” refers to a flexible polypeptide connector region (also referred to herein as “hinge”) that may be included within the CAR ectodomain (or extracellular domain) providing structural flexibility to flanking polypeptide regions. The hinge region may consist of natural or synthetic polypeptides. It will be appreciated by those skilled in the art that hinge regions may improve the function of the CAR by promoting optimal positioning of the DLL3 binding domain in relationship to the portion of the antigen recognized by the same. It will be appreciated that, in some embodiments, the hinge region may not be required for optimal CAR activity. In other embodiments a beneficial hinge region comprising a short sequence of amino acids promotes CAR activity by facilitating flexibility of the antigen binding domain or antibody. The sequence encoding the hinge region may be positioned between the antigen recognition moiety (e.g., an anti-DLL3 scFv) and the transmembrane domains. The hinge sequence can be any moiety or sequence derived or obtained from any suitable molecule. In one embodiment, for example, the hinge sequence is derived from the human CD8α molecule or a CD28 molecule. A “hinge region” derived from an immunoglobulin (e.g., IgGI) is generally defined as stretching from Glu216 to Pro230 of human IgG1. Hinge regions of other IgG isotypes may be aligned with the IgG1 sequence by placing the first and last cysteine residues forming inter-heavy chain disulfide (S—S) bonds in the same positions. In other embodiments the hinge region may be of natural occurrence or non-natural occurrence, including but not limited to an altered hinge region as described in U.S.P.N. 5,677,425. Of course, when certain binding domains such as (Fab′)₂ or an intact antibody are used in the CAR it will naturally follow that the corresponding hinge region will be included.

In other selected embodiments the hinge region can include complete hinge region derived from an antibody of a different class or subclass from that of the CH1 domain. The term “hinge region” can also include regions derived from human CD8a (aka CD8a) molecule or a CD28 molecule and any other receptors that provide a similar function in providing flexibility to flanking regions. The hinge region can have a length of from about 4 amino acids to about 50 amino acids, e.g., from about 4 aa to about 10 aa, from about 10 aa to about 15 aa, from about aa to about 20 aa, from about 20 aa to about 25 aa, from about 25 aa to about 30 aa, from about 30 aa to about 40 aa, or from about 40 aa to about 50 aa. Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

Those skilled in the art will appreciate that compatible hinge regions are well known and, as such, operable embodiments may readily be selected and incorporated in the DLL3 CARs of the instant invention.

C. Transmembrane/Spacer Domain

As alluded to above, the DLL3 CARs of the instant invention preferably comprise a transmembrane domain that is interposed between the extracellular DLL3 binding domain and/or hinge region, and the intracellular or cytoplasmic signaling domain. For the purposes of the instant discussion the term “transmembrane domain” will be used with the understanding that while it always includes amino acid residues that are physically buried in the lipid bilayer of a cellular membrane, it may include support or “spacer domains” that can extend beyond either side of the cell membrane. Those of skill in the art can readily distinguish between the functional aspects of the CAR components and easily determine what constitutes a compatible transmembrane domain in view of the instant disclosure.

It will be appreciated that the transmembrane domain may be derived from a natural polypeptide, or may be artificially designed. Compatible transmembrane domains may be derived from any membrane-binding or transmembrane protein which may be modified or truncated as necessary. For example, transmembrane domains derived from a T cell receptor a or P chain, a Fc region of an IgG (such as IgG4), a CD3ζ chain, CD28, CD3ε, CD45, CD4, CD5, CD8 (e.g. CD8a aka CD8α), CD9, CD16, CD22, CD33, CD37, CD64, CD80, CD86, CD134, CD137, ICOS, CD154 or a GITR are all compatible with various embodiments of the disclosed DLL3 CAR constructs. In certain embodiments it is preferred to employ the transmembrane domain of CD8ζ, FcRη, FcεR1-γ and −β, MB1 (Igα), B29 or CD3-γ, ζ, or ε, in order to retain physical association with other members of the receptor complex. Compatible artificial transmembrane domains may comprise various polypeptide sequences incorporating high levels of hydrophobic residues such as leucine and valine. In other preferred embodiments the transmembrane domain may comprise a triplet of phenylalanine, tryptophan and valine which is located at each end of the synthetic transmembrane domain.

Certain embodiments of the invention will comprise transmembrane domains having a spacer. In the DLL3 CARs of the present invention, a “spacer domain” or “spacer region” is an amino acid sequence that can be arranged between an extracellular functional domain (e.g., the antigen binding domain or the hinge region if included) and the transmembrane domain, or between the intracellular signaling domain and the transmembrane domain. The spacer domain means any oligopeptide or polypeptide that serves to link the transmembrane domain with the extracellular domain and/or the transmembrane domain with the intracellular domain, with the intent to optimally position these elements within the CAR polypeptide for efficient CAR function. The spacer domain comprises up to 300 amino acids, preferably 10 to 100 amino acids, and most preferably 25 to 50 amino acids. The spacer domain preferably has a sequence that promotes binding of the DLL3 CAR with DLL3 and enhances transmembrane signaling into a cell. Examples of amino acids that are expected to promote the binding include cysteine, a charged amino acid, and serine and threonine in a potential glycosylation site, and these amino acids can be used as an amino acid constituting the spacer domain. In preferred embodiments the spacer may comprise all or part of an antibody constant region (e.g., IgG1 CH or CL) which may optionally dimerize.

Other compatible spacers include glycine polymers (G)ₙ, glycine-serine polymers, glycine-alanine polymers, alanine-serine polymers, and other flexible spacers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains.

Those skilled in the art will appreciate that compatible transmembrane domains are well known in the art and, as such, operable embodiments may readily be selected and incorporated in the DLL3 CARs of the instant invention.

D. Intracellular Signaling Domain

In addition to the extracellular DLL3 binding domain and the transmembrane domain, the DLL3 CARs of the instant invention will incorporate an intracellular or cytoplasmic domain comprising at least one signaling and/or T cell activating moiety. The intracellular signaling domain used in the present invention is a molecule that can transmit one or more signals into a cell when the extracellular domain present within (or non-covalently associated with) the same molecule binds to (interacts with) DLL3. The binding of DLL3 triggers a signal that passes along the CAR and is transmitted intracellularly to activate the sensitized lymphocyte. This lymphocyte activation triggers the desired immune response that results in the elimination of the target cell.

The two signal theory of T-lymphocyte activation proposes that two signals are required to efficiently activate T-cells: first, antigenic peptides presented in the context of an MHC molecule interact with the alpha:beta chain heterodimer of the TCR, leading to conformational changes that result in activation of a signal from the cytoplasmic domains found in protein components of the TCR complex; and second, transmission of a signal from the cytoplasmic domain of a single or several costimulatory molecules as they interact with their cognate ligands on the cell presenting the peptide:MHC complex. More specifically it is known that a signal generated only via a TCR complex may be insufficient to activate a T cell, and a secondary or costimulating signal is also required to avoid a state of T-cell inactivity known an anergy. Natural T cell-activation is transmitted by two different kinds of cytoplasmic signaling sequences, that is, a sequence for initiating antigen-dependent primary activation via a TCR complex (primary cytoplasmic signaling sequence) and a sequence for acting antigen-independently to provide a secondary or costimulating signal (secondary cytoplasmic signaling sequence). In a preferable aspect, the DLL3 CAR of the present invention comprises the primary cytoplasmic signaling sequence and/or the secondary cytoplasmic signaling sequence as the CAR endodomain.

In general, signaling motifs found in the cytoplasmic domains of immune system receptors may be activating or inhibitory. The primary cytoplasmic signaling sequence that stimulates the activation may comprise a signal transduction motif known as an immunoreceptor tyrosine-based activation motif (ITAM) [Nature, vol. 338, pp. 383-384 (1989)]. On the other hand, the primary cytoplasmic signaling sequence that acts in an inhibitory way comprises a signal transduction motif known as an immunoreceptor tyrosine-based inhibition motif (ITIM). In the present invention, an intracellular domain having an ITAM or an ITIM can be used.

The primary cytoplasmic signaling sequence that transmits the first stimulating signal for T-cell activation from the native TCR complex is an ITAM found in the CD3ζ chain, but it is known that other ITAMs may also be employed to transmit positive primary activating signal. Examples of the intracellular domain having an ITAM that can be used in the present invention include intracellular domains having ITAM derived from CD3ζ, FcRγ, FcRβ, CD3γ, CD3 δ, CD3ε, CD5, CD22, CD79a, CD79b, and CD66d. Specifically, examples of the ITAM include peptides having sequences of amino acid numbers 51 to 164 of CD3ζ (NCBI RefSeq: NP_932170.1), amino acid numbers 45 to 86 of FcεRIγ (NCBI RefSeq: NP_004097.1), amino acid numbers 201 to 244 of FcεRIβ (NCBI RefSeq: NP_000130.1), amino acid numbers 139 to 182 of CD3γ (NCBI RefSeq: NP_000064.1), amino acid numbers 128 to 171 of CD3 δ (NCBI RefSeq: NP_000723.1), amino acid numbers 153 to 207 of CD3c (NCBI RefSeq: NP_000724.1), amino acid numbers 402 to 495 of CD5 (NCBI RefSeq: NP_055022.2), amino acid numbers 707 to 847 of 0022 (NCBI RefSeq: NP_001762.2), amino acid numbers 166 to 226 of CD79a (NCBI RefSeq: NP_001774.1), amino acid numbers 182 to 229 of CD79b (NCBI RefSeq: NP_000617.1), and amino acid numbers 177 to 252 of CD66d (NCBI RefSeq: NP_001806.2), and their variants having the same function as these peptides have. The amino acid number based on amino acid sequence information of NCBI RefSeq ID or GenBank described herein is numbered based on the full length of the precursor (comprising a signal peptide sequence etc.) of each protein.

The secondary, costimulatory signal may come from the cytoplasmic domain of a variety of costimulatory molecules, the best characterized of which is CD28. CD28 is expressed on T-cells and is the receptor for CD80 (B7.1) and CD86 (B7.2). However, other costimulatory molecules include, but are not limited to the CD27 molecule, the CD137/4-1BB molecule, the CD134/OX40 molecule, and other intracellular signaling molecules known in the art. CD134/OX40 is known to enhance T-cell clonal expansion, likely by suppressing apoptosis, and may play a role in the establishment of memory cells. 4-1BB, also known as CD137, transmits a potent costimulatory signal to T-cells, promoting differentiation and enhancing long-term survival of T lymphocytes. As each of these costimulatory molecules activates different intracellular signaling pathways and may have differing effects in different populations of T-lymphocytes, domains from, one, several, or each may be included in the endodomain of the CAR in order to maximize T-cell activation and other desired properties of the CAR. In a preferred embodiment, the CD28, CD27, 4-1BB, and OX40 molecules are human. Examples of the intracellular domain comprising a secondary cytoplasmic signaling sequence that can be used in the present invention include sequences derived from CD2, CD4, CD5, CD8α, CD83, CD28, CD134, CD137 (4-1BB), ICOS, and CD154. Specific examples thereof include peptides having sequences of amino acid numbers 236 to 351 of CD2 (NCBI RefSeq: NP-001758.2), amino acid numbers 421 to 458 of CD4 (NCBI RefSeq: NP-000607.1), amino acid numbers 402 to 495 of CD5 (NCBI RefSeq: NP-055022.2), amino acid numbers 207 to 235 of CD8a (NCBI RefSeq: NP-001759.3), amino acid numbers 196 to 210 of CD83 (GenBank: AAA35664.1), amino acid numbers 181 to 220 (SEQ ID NO: 25) of CD28 (NCBI RefSeq: NP-006130.1), amino acid numbers 214 to 255 of CD137 (4-1BB, NCBI RefSeq: NP-001552.2), amino acid numbers 241 to 277 of CD134 (OX40, NCBI RefSeq: NP-003318.1), and amino acid numbers 166 to 199 of ICOS (NCBI RefSeq: NP-036224.1), and their variants having the same function as these peptides have.

The signaling/activating domain(s) of the DLL3 CAR encoded by the disclosed nucleic acid sequence can comprise any one of aforementioned signaling domains and any one or more of the aforementioned intercellular T-cell activating domains in any combination. For example, the inventive nucleic acid sequence can encode a CAR comprising a CD28 signaling domain and intracellular T-cell activating domains of CD28 and CD34. Alternatively, for example, the nucleic acid sequences of the invention can encode a CAR comprising a CD8α signaling domain and T cell signaling domains of CD28, CD3ζ, the Fc receptor gamma (FcRγ) chain, and/or 4-1BB. As shown in the Examples below selected embodiments may comprise a 4-1BB costimulatory region along with a CD3ζ cytoplasmic region or variants thereof.

Those skilled in the art will appreciate that each of the aforementioned signaling/stimulatory domains are compatible with the instant invention and may be used effectively (alone or preferably in combination) with the disclosed DLL3 CARs. Accordingly, each of the aforementioned moieties, in any combination or configuration are expressly contemplated as being within the scope of the instant invention as components of the intracellular/cytoplasmic domain.

### V. CAR Nucleic Acids and Vectors

The invention provides an isolated or purified nucleic acid sequence encoding an anti-DLL3 chimeric antigen receptor, wherein the CAR preferably comprises an extracellular DLL3 binding domain (e.g., a scFv), a transmembrane domain and an intracellular signaling domain (e.g., a T-cell activation moiety). As used herein “nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to methylated and/or capped polynucleotides.

By “isolated” is meant the removal of a nucleic acid from its natural environment. By “purified” is meant that a given nucleic acid, whether one that has been removed from nature (including genomic DNA and mRNA) or synthesized (including cDNA) and/or amplified under laboratory conditions, has been increased in purity, wherein “purity” is a relative term, not “absolute purity.” It is to be understood, however, that nucleic acids and proteins may be formulated with diluents or adjuvants and still for practical purposes be isolated. For example, nucleic acids typically are mixed with an acceptable carrier or diluent when used for introduction into cells.

As described herein and shown in the appended Examples, nucleic acid sequences compatible with the invention can be generated using methods known in the art. For example, nucleic acid sequences, polypeptides, and proteins can be recombinantly produced using standard recombinant DNA methodology (see, e.g., Sambrook et al., Molecular Cloning: A Laboratory Manual, 3rd ed., Cold Spring Harbor Press, Cold Spring Harbor, N.Y. 2001). Further, a synthetically produced nucleic acid sequence encoding the DLL3 CAR can be isolated and/or purified from a source, such as a CHO cell, plant, a bacterium, an insect, or a mammal, e.g., a rat, a human, etc. Methods of isolation and purification are well known in the art. Alternatively, the nucleic acid sequences described herein can be commercially synthesized. In this respect, the inventive nucleic acid sequence can be synthetic, recombinant, isolated, and/or purified.

A nucleic acid sequence of the invention can encode a DLL3 CAR of any length, i.e., the CAR can comprise any number of amino acids, provided that the CAR retains its biological activity, e.g., the ability to specifically bind to antigen and treat or prevent disease in a mammal, etc. For example, the CAR can comprise 50 or more, 60 or more, 100 or more, 250 or more, or 500 or more amino acids. Preferably, the CAR is about 50 to about 700 amino acids (e.g., about 70, about 80, about 90, about 150, about 200, about 300, about 400, about 550, or about 650 amino acids), about 100 to about 500 amino acids (e.g., about 125, about 175, about 225, about 250, about 275, about 325, about 350, about 375, about 425, about 450, or about 475 amino acids), or a range defined by any two of the foregoing values.

Included in the scope of the invention are nucleic acid sequences that encode functional portions of the DLL3 CAR described herein. The term “functional portion,” when used in reference to a CAR, refers to any part or fragment of the CAR of the invention, which part or fragment retains the biological activity of the CAR of which it is a part (the parent CAR). Functional portions encompass, for example, those parts of a CAR that retain the ability to recognize target cells or provide an immunomodulatory signal, or treat a disease, to a similar extent, the same extent, or to a higher extent, as the parent CAR. In reference to a nucleic acid sequence encoding the parent DLL3 CAR, a nucleic acid sequence encoding a functional portion of the CAR can encode a protein comprising, for example, about 10%, 25%, 30%, 50%, 68%, 80%, 90%, 95%, or more, of the parent CAR. In this regard compatible nucleic acid sequences can encode a functional portion of a CAR that contains additional amino acids at the amino or carboxy terminus of the portion, or at both termini, which additional amino acids are not found in the amino acid sequence of the parent CAR. Desirably, the additional amino acids do not interfere with the biological function of the functional portion, e.g., recognize target cells, detect cancer, treat or prevent cancer, etc. More desirably, the additional amino acids enhance the biological activity of the CAR, as compared to the biological activity of the parent CAR.

The invention also provides nucleic acid sequences encoding functional variants of the DLL3 CAR. The term “functional variant,” as used herein, refers to a CAR, a polypeptide, or a protein having substantial or significant sequence identity or similarity to the CAR encoded by the disclosed nucleic acid sequences, which functional variant retains the DLL3 binding capacity of the CAR of which it is a variant. Functional variants encompass, for example, those variants of the CAR described herein (the parent CAR) that retain the ability to recognize DLL3 positive target cells to a similar extent, the same extent, or to a higher extent, as the parent CAR. In reference to a nucleic acid sequence encoding the parent CAR, a nucleic acid sequence encoding a functional variant of the CAR can be for example, about 10% identical, about 25% identical, about 30% identical, about 50% identical, about 65% identical, about 80% identical, about 90% identical, about 95% identical, or about 99% identical to the nucleic acid sequence encoding the parent CAR.

Regardless of the precise form of the DLL3 CAR it will be appreciated that the nucleic acids of the present invention may be used for ex vivo transformation of selected host cells (e.g., lymphocytes) or introduced directly into the subject for in vivo gene therapy. In each case the disclosed nucleic acids may be combined with a substance that promotes transference of a nucleic acid into a cell, for example, a reagent for introducing a nucleic acid such as a liposome or a cationic lipid, in addition to other excipients disclosed herein. In certain preferred embodiments the nucleic acids of the instant invention will be combined with, or integrated into, a vector is suitable for in vivo gene therapy.

Accordingly, in conjunction with the foregoing the present invention provides compositions comprising DLL3 CAR nucleic acids which, together with a pharmaceutically acceptable carrier, may be used as an active ingredient (e.g., in in vivo gene therapy) or to generate sensitized lymphocytes. Suitable pharmaceutically acceptable additives are well known to a person skilled in the art. Examples of the pharmaceutically acceptable additives or excipients include phosphate buffered saline (e.g. 0.01 M phosphate, 0.138 M NaCl, 0.0027 M KCl, pH 7.4), an aqueous solution containing a mineral acid salt such as a hydrochloride, a hydrobromide, a phosphate, or a sulfate, saline, a solution of glycol or ethanol, and a salt of an organic acid such as an acetate, a propionate, a malonate or a benzoate. An adjuvant such as a wetting agent or an emulsifier, and a pH buffering agent can also be used. Compositions of the present invention can be formulated into a known form suitable for parenteral administration, for example, injection or infusion. Further, such compositions may comprise formulation additives such as a suspending agent, a preservative, a stabilizer and/or a dispersant, and a preservation agent for extending a validity term during storage. Further the composition may be in a dry form for reconstitution with an appropriate sterile liquid prior to use.

In addition to the nucleic acid sequence encoding the DLL3 CAR, compatible vectors preferably comprise expression control sequences, such as promoters, enhancers, polyadenylation signals, transcription terminators, internal ribosome entry sites (IRES), and the like, that provide for the expression of the nucleic acid sequence in a host cell. In this regard a large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system, the Ecdysone inducible system, the T-REX™ system (Invitrogen, Carlsbad, Calif.), LACSWITCH™ System (Stratagene, San Diego, Calif.), and the Cre-ERT tamoxifen inducible recombinase system. In addition the DLL3 CAR may be associated with a gene that can be a marker for confirming expression of the nucleic acid (e.g. a drug resistance gene, a gene encoding a reporter enzyme, or a gene encoding a fluorescent protein).

In certain embodiments the nucleic acid encoding the DLL3 CAR, along with any control elements, can preferably be inserted into a vector that can then be introduced into a selected cell to provide the disclosed DLL3 sensitized lymphocytes. In preferred embodiments the vector can be, for example, a plasmid, a transposon, a cosmid or a viral vector (e.g., phage, retroviral, lentiviral or adenoviral). For example, a virus vector such as a retrovirus vector (including an oncoretrovirus vector, a lentivirus vector, and a pseudo type vector), an adenovirus vector, an adeno-associated virus (AAV) vector, a simian virus vector, a vaccinia virus vector or a sendai virus vector, an Epstein-Barr virus (EBV) vector, and a HSV vector can be used.

More generally the terms “vector”, “cloning vector” and “expression vector” mean the vehicle by which a DNA or RNA sequence (e.g., a foreign gene encoding a DLL3 CAR) can be introduced into a host cell, so as to transform the host and promote expression (e.g. transcription and translation) of the introduced sequence. It will be appreciated that the introduced gene or sequence may include regulatory or control sequences, such as start, stop, promoter, signal, secretion, or other sequences used by a cell's genetic machinery. As described herein compatible vectors are well known in the art and include plasmids, transposons, phages, viruses, etc. The vector may then be used to transform the selected lymphocytes (autologous or allogeneic) to provide the disclosed sensitized lymphocytes. For the purposes of the instant disclosure the term “transform” or “transformation” will be used in its most general sense and shall be held to mean the introduction of a heterologous gene, DNA or RNA sequence to a host cell (prokaryotic or eukaryotic), so that the host cell will express the introduced gene or sequence to produce a desired substance, typically a protein or enzyme coded by the introduced gene or sequence. Exemplary methods of cell transformation compatible with the invention comprise transfection and transduction. As used herein the term “transfection” means the introduction of a foreign nucleic acid or gene into a cell (prokaryotic or eukaryotic) using physical or chemical means while the term “transduction” means the introduction of a foreign nucleic acid or gene into a cell (prokaryotic or eukaryotic) through use of a viral vector.

In terms of transduction phage or viral vectors can be introduced into host cells, preferably after growth of infectious particles in suitable packaging cells, many of which are commercially available. Compatible transduction methods and packaging cells are set forth in the Examples below and would be readily discernable to the skilled artisan in view of the instant disclosure.

By way of example, when a retrovirus vector is to be used, compositions compatible with the teachings herein can be generated by selecting a suitable packaging cell based on a LTR sequence and a packaging signal sequence possessed by the vector and preparing a retrovirus particle using the packaging cell. Examples of the packaging cell include PG13 (ATCC CRL-10686), PA317 (ATCC CRL-9078), GP+E-86 and GP+envAm-12, and Psi-Crip. A retrovirus particle can also be prepared using a 293 cell or a 293T cell having high transfection efficiency. Many kinds of retrovirus vectors produced based on retroviruses and packaging cells that can be used for packaging of the retrovirus vectors are widely commercially available from many companies. Similar systems are also commercially available for the fabrication of compatible lentiviral vectors in accordance with the teachings herein. Such vectors may be used to transduce selected lymphocyte populations to provide the desired DLL3 sensitized lymphocytes.

In addition, non-viral packaging vector systems can also be used in the present invention in combination with a liposome and a condensing agent such as a cationic lipid as described in WO 96/10038, WO 97/18185, WO 97/25329, WO 97/30170 and WO 97/31934 (which are incorporated herein by reference).

Similarly, many methods of transfection are compatible with the instant invention and may be used in conjunction with the teachings herein to provide the desired compositions. As discussed, transfection typically refers to the introduction of one or more exogenous polynucleotides into a host cell by using physical or chemical methods. Many transfection techniques are known in the art and include, for example, calcium phosphate DNA co-precipitation; DEAE-dextran; electroporation; cationic liposome-mediated transfection; tungsten particle-facilitated microparticle bombardment; and strontium phosphate DNA co-precipitation. Additionally, electroporation, sonoporation, impalefection, optical transfection and hydro dynamic delivery comprise some non-chemical based gene transfection methods compatible with the instant invention.

Regardless of which methodology is selected to effect transformation, it will be appreciated that the DLL3 CAR nucleic acid constructs and vectors may be used to generate the disclosed sensitized lymphocytes.

### VI. Host Cells

A vector comprising a nucleic acid encoding the DLL3 CAR can be introduced into any host cell that is capable of carrying and/or expressing the CAR protein, including any suitable prokaryotic or eukaryotic cell. Particularly compatible methods of transformation comprise the use of lentiviral, and retroviral systems along with transposons and naked RNA. Preferred host cells are those that can be easily and reliably grown, have reasonably fast growth rates, have well characterized expression systems, and can be transformed or transfected easily and efficiently.

As used herein, the term “host cell” refers to any type of cell that can contain the expression vector. The host cell can be a eukaryotic cell (e.g., plant, animal, fungi, or algae), a prokaryotic cell (e.g., bacteria or protozoa) or a viral or retroviral vector. The host cell can be a cultured or “off-the-shelf” cell or a primary cell (i.e., isolated directly from a subject). The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5α cell. For purposes of producing a recombinant CAR, the host cell can be a mammalian cell. The host cell preferably is a human cell. The host cell can be of any cell type, can originate from any type of tissue, and can be of any developmental stage. For example, a cell collected, isolated, purified or induced from a body fluid, a tissue or an organ such as blood (peripheral blood, umbilical cord blood etc.) or bone marrow can be used. A peripheral blood mononuclear cell (PBMC), an immune cell [a dendritic cell, a B cell, a hematopoietic stem cell, a macrophage, a monocyte, a NK cell or a hematopoietic cell (a neutrophil, a basophil)], an umbilical cord blood mononuclear cell, a fibroblast, a precursor adipocyte, a hepatocyte, a skin keratinocyte, a mesenchymal stem cell, an adipose stem cell, various cancer cell strains, or a neural stem cell can be used. In particularly preferred embodiments the host cell can be a peripheral blood lymphocyte (PBL), a peripheral blood mononuclear cell (PBMC), or a natural killer (NK) cell. In selected embodiments the host cell is a natural killer (NK) cell. In other preferred embodiments the host cell will be a T-cell. Methods for selecting suitable mammalian host cells and methods for transformation, culture, amplification, screening, and purification of cells are known in the art.

The invention provides an isolated host cell that expresses nucleic acid sequence encoding the DLL3 CARs described herein or compositions of the same. In particularly preferred embodiments the host cell comprises a lymphocyte which is transformed into a DLL3 sensitized lymphocyte upon expression of the disclosed CARs. In one embodiment, the host cell is a T-cell. The T-cell of the invention can be any T-cell, such as a cultured T-cell (e.g., a primary T-cell, or a T-cell from a cultured T-cell line, or a T-cell obtained from a mammal). If obtained from a mammal, the T-cell can be obtained from numerous sources, including but not limited to blood, bone marrow, lymph node, the thymus, or other tissues or fluids. T-cells can also be enriched for or purified. The T-cell preferably is a human T-cell (e.g., isolated from a human). The T-cell can be of any developmental stage, including but not limited to, a CD4+/CD8+ double positive T-cell, a CD4+ helper T-cell, e.g., Th1 and The cells, a CD8+ T-cell (e.g., a cytotoxic T-cell), a tumor infiltrating cell, a memory T-cell, a naïve T-cell, and the like. In one embodiment, the T-cell is a CD8+ T-cell or a CD4+ T-cell. T-cell lines are available from commercial sources (e.g., the American Type Culture Collection and the German Collection of Microorganisms and Cell Cultures) and include, for example, Jurkat cells (ATCC TIB-152), Sup-T1 cells (ATCC CRL-1942), RPMI 8402 cells (DSMZ ACC-290), Karpas 45 cells (DSMZ ACC-545), and derivatives thereof.

In another embodiment, the host cell is a natural killer (NK) cell. NK cells are a type of cytotoxic lymphocyte that plays a role in the innate immune system. NK cells are defined as large granular lymphocytes and constitute the third kind of cells differentiated from the common lymphoid progenitor which also gives rise to B and T lymphocytes (see, e.g., Immunobiology, 5th ed., Janeway et al., eds., Garland Publishing, New York, N.Y. (2001)). NK cells differentiate and mature in the bone marrow, lymph node, spleen, tonsils, and thymus. Following maturation. NK cells enter into the circulation as large lymphocytes with distinctive cytotoxic granules. NK cells are able to recognize and kill some abnormal cells, such as, for example, some tumor cells and virus-infected cells, and are thought to be important in the innate immune defense against intracellular pathogens. As described above with respect to T-cells, the NK cell can be any NK cell, such as a cultured NK cell, e.g., a primary NK cell, or an NK cell from a cultured NK cell line, or an NK cell obtained from a mammal. If obtained from a mammal, the NK cell can be obtained from numerous sources, including but not limited to blood, bone marrow, lymph node, the thymus, or other tissues or fluids. NK cells can also be enriched for or purified. The NK cell preferably is a human NK cell (e.g., isolated from a human). NK cell lines are available from commercial sources (e.g., the American Type Culture Collection) and include, for example, NK-92 cells (ATCC CRL-2407), NK92MI cells (ATCC CRL-2408), and derivatives thereof.

In autologous adoptive immunotherapy, a patient's circulating lymphocytes, or tumor infiltrated lymphocytes, are isolated (e.g., by apheresis) in vitro, preferably activated or stimulated by lymphokines such as IL-2 and then transduced with nucleic acids encoding a DLL3 CAR construct. Following transduction the autologous sensitized lymphocytes are preferably expanded using cytokine support as known in the art and readministered to the patient. To achieve this, one would administer to an animal, or human patient, an immunologically effective amount of activated lymphocytes genetically modified to express a DLL3 CAR gene as described herein. In such autologous procedures the activated lymphocytes (i.e., DLL3 sensitized lymphocytes) are the patient's own cells that most preferably were earlier isolated from a blood or tumor sample and activated and expanded in vitro. In some aspects of the present invention T lymphocytes or NK cells from a patient having cancer would be isolated and transduced with SCT1-h16.15 polynucleotides (e.g., see Example 10 below) so that the DLL3 CAR is expressed on the cell surface of the T cell or NK cell. The modified cells would then be readministered into the patient to target and kill the tumor cells (see generally FIG. 5).

Other preferred aspects of the invention comprise allogeneic transplants of DLL3 sensitized lymphocytes. In such embodiments the disclosed DLL3 CARs may be introduced (e.g., through transduction) into lymphocytes obtained from a source other than the subject to be treated. Some aspects of the instant invention comprise the use of allogeneic lymphocytes obtained from a donor that has been immunologically matched with the recipient to reduce the chance of rejection. In other aspects the disclosed CARs will be introduced into “off-the-shelf” allogeneic lymphocytes (see PMID: 26183927 which is incorporated herein by reference) that have been modified to facilitate transplantation and generate the appropriate immune response upon contact with the target cell. It will be appreciated that the use of such prefabricated allogeneic lymphocyte preparations may provide several advantages in terms of preparing the pharmaceutically active sensitized lymphocytes and reducing the chances of patient rejection.

It will also be appreciated that the selected host cells can be expanded in vitro before or after transformation with the DLL3 CAR. Methods for expanding the selected cell populations are well known in the art and several commercial kits compatible with the instant invention are available. In this regard T cells and or NK cells may be expanded in vitro to provide more robust dosing options. For example, in accordance with the present invention NK cells may be preferentially expanded by exposure to cells that lack or poorly express major histocompatibility complex I and/or II molecules and which have been genetically modified to express membrane bound IL-15 and 4-1BB ligand (CDI37L). Such cell lines include, but are not necessarily limited to, K562 (ATCC, CCL 243), and the Wilms tumor cell line HFWT, the uterine endometrium tumor cell line HHUA, the melanoma cell line HMV-II, the hepatoblastoma cell line HuH-6, the lung small cell carcinoma cell lines Lu-130 and Lu-134-A, the neuroblastoma cell lines NB 19 and N1369, the embryonal carcinoma cell line from testis NEC 14, the cervix carcinoma cell line TCO-2, and the bone marrow-metastated neuroblastoma cell line TNB 1. Preferably the cell line used lacks or poorly expresses both MHC I and II molecules, such as the K562 and HFWT cell lines. Similar techniques allow for the expansion of selected T cell populations. In this regard some processes employ anti-CD3 plus autologous or allogeneic feeder cells and high doses of IL-2. Other processes use IL-7, 11-15, IL-21 or combinations thereof for expansion and stimulation of T cells. It will be appreciated that each of the aforementioned processes, along with any process that provides the desired number of DLL3 sensitized lymphocytes, is compatible with the instant invention.

### VII. Formulation and Administration of DLL3 Sensitized Lymphocytes

As set forth herein the selected host cells can be expanded in vitro for use in adoptive cellular immunotherapy comprising autologous or allogeneic DLL3 sensitized lymphocytes. In this regard the compositions and methods of this invention can be used to generate a population of sensitized lymphocytes that preferably deliver both primary and costimulatory signals for use in the treatment of cancer and, by way of example, the treatment of lung cancer including small cell lung cancer, melanoma, breast cancer, prostate cancer, colon cancer, renal cell carcinoma, ovarian cancer, neuroblastoma, rhabdomyosarcoma, leukemia and lymphoma. The compositions and methods described in the present invention may be used in conjunction with other types of therapy for cancer, such as chemotherapy, surgery, radiation, gene therapy, and so forth.

The DLL3 sensitized lymphocytes or host cells are preferably administered to a subject in the form of a pharmaceutical composition comprising one or more pharmaceutically acceptable carriers. In particularly preferred embodiments the disclosed pharmaceutical compositions will comprise a population of T cells or NK cells (autologous or allogeneic) that express the DLL3 CAR. Besides such host cells pharmaceutical compositions of the invention can comprise other pharmaceutically active agents or drugs, such as chemotherapeutic agents (e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.) or adjuvant therapies that further stimulate the immune response. In a preferred embodiment, the pharmaceutical composition comprises isolated T cells or NK cells which express the disclosed DLL3 CARs and more preferably a population of sensitized T cells or NK cells which express the disclosed DLL3 CARs. In addition such compositions may comprise a pharmaceutically acceptable buffers, preservatives, excipients, etc. as is well known in the art.

Alternatively, nucleic acid sequences encoding the DLL3 CAR, or vectors comprising a DLL3 CAR-encoding nucleic acid sequence, can be formulated into a pharmaceutical composition and administered directly to the patient. In such embodiments vector systems comprising viral vector host cells (e.g., lentiviral systems or retroviral systems) or directed artificial viral envelopes are preferred. Such vectors allow for the in vivo generation of DLL3 sensitized lymphocytes which can then induce the desired anti-tumor immune response.

In any event the DLL3 CAR host cells of the invention and any co-reagents can be formulated in various ways using art recognized techniques. In some embodiments, the therapeutic compositions of the invention can be administered neat or with a minimum of additional components while others may optionally be formulated to contain suitable pharmaceutically acceptable carriers. As used herein, “pharmaceutically acceptable carriers” comprise excipients, vehicles, adjuvants and diluents that are well known in the art and can be available from commercial sources for use in pharmaceutical preparation (see, e.g., Gennaro (2003) Remington: The Science and Practice of Pharmacy with Facts and Comparisons: Drugfacts Plus, 20th ed., Mack Publishing; Ansel et al. (2004) Pharmaceutical Dosage Forms and Drug Delivery Systems, 7^{th} ed., Lippencott Williams and Wilkins; Kibbe et al. (2000) Handbook of Pharmaceutical Excipients, 3^{rd} ed., Pharmaceutical Press.)

Suitable pharmaceutically acceptable carriers typically comprise substances that are relatively inert and can facilitate administration of the sensitized lymphocyte or host cell or can aid processing of the same into preparations that are pharmaceutically optimized for delivery to the site of action. Such pharmaceutically acceptable carriers include agents that can alter the form, consistency, viscosity, pH, tonicity, stability, osmolarity, pharmacokinetics, protein aggregation or solubility of the formulation and include buffering agents, wetting agents, emulsifying agents, diluents, encapsulating agents and skin penetration enhancers. Certain non-limiting examples of carriers include saline, buffered saline, dextrose, arginine, sucrose, water, glycerol, ethanol, sorbitol, dextran, sodium carboxymethyl cellulose and combinations thereof. Sensitized lymphocytes for systemic administration may be formulated for enteral, parenteral or topical administration. Indeed, all three types of formulation may be used simultaneously to achieve systemic administration of the active ingredient. Excipients as well as formulations for parenteral and nonparenteral drug delivery are well known in the art.

Formulations suitable for parenteral administration of DLL3 sensitized lymphocytes (e.g., by injection or infusion), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additionally contain other pharmaceutically acceptable carriers, such as anti-oxidants, buffers, preservatives, stabilizers, bacteriostats, suspending agents, thickening agents, and solutes that render the formulation isotonic with the blood (or other relevant bodily fluid) of the intended recipient. Examples of excipients include, for example, water, alcohols, polyols, glycerol, vegetable oils, and the like. Examples of suitable isotonic pharmaceutically acceptable carriers for use in such formulations include sodium chloride injection, Ringer's Solution, or Lactated Ringer's Injection.

Methods of introducing cellular components are also known in the art and include procedures such as those exemplified in U.S.P.Ns. 4,844,893 and 4,690,915. The amount of DLL3 sensitized lymphocytes (e.g., T cells or NK cells) used can vary between in vitro and in vivo uses, as well as with the amount and type of the target cells. The amount administered will also vary depending on the condition of the patient and should be determined by the practitioner after considering all appropriate factors.

The particular dosage regimen of DLL3 sensitized lymphocytes, i.e., dose, timing and repetition, will depend on the particular individual, as well as empirical considerations such as pharmacokinetics (e.g., half-life, clearance rate, etc.). For example, individuals may be given incremental dosages of sensitized lymphocytes produced as described herein. In selected embodiments the dosage may be gradually increased or reduced or attenuated based respectively on empirically determined or observed side effects or toxicity. Determination of the frequency of administration may be made by persons skilled in the art, such as an attending physician based on considerations of the condition and severity of the condition being treated, age and general state of health of the subject being treated and the like. Frequency of administration may be adjusted over the course of therapy based on assessment of the efficacy of the selected composition and the dosing regimen. Such assessment can be made on the basis of markers of the specific disease, disorder or condition. In embodiments where the individual has cancer, these include direct measurements of tumor size via palpation or visual observation; indirect measurement of tumor size by x-ray or other imaging techniques; an improvement as assessed by direct tumor biopsy and microscopic examination of a tumor sample; the measurement of an indirect tumor marker (e.g., DLL3 for SCLC) or an antigen identified according to the methods described herein; reduction in the number of proliferative or tumorigenic cells, maintenance of the reduction of such neoplastic cells; reduction of the proliferation of neoplastic cells; or delay in the development of metastasis.

In view of the instant disclosure the DLL3 CAR may be administered on a specific schedule. Generally, an effective dose of the sensitized lymphocytes is administered to a subject one or more times. More particularly, an effective dose of the DLL3 CAR is administered to the subject once a month, more than once a month, or less than once a month. In certain embodiments, the effective dose of the DLL3 sensitized lymphocytes may be administered multiple times, including for periods of at least a month, at least six months, at least a year, at least two years or a period of several years. In yet other embodiments, several days (2, 3, 4, 5, 6 or 7), several weeks (1, 2, 3, 4, 5, 6, 7 or 8) or several months (1, 2, 3, 4, 5, 6, 7 or 8) or even a year or several years may lapse between administration of the DLL3 sensitized lymphocytes.

In certain preferred embodiments the course of treatment involving DLL3 CAR will comprise multiple doses of the selected sensitized lymphocytes over a period of weeks or months. More specifically, DLL3 sensitized lymphocytes of the instant invention may administered once every day, every two days, every four days, every week, every ten days, every two weeks, every three weeks, every month, every six weeks, every two months, every ten weeks or every three months. In this regard it will be appreciated that the dosages may be altered or the interval may be adjusted based on patient response and clinical practices.

A typical amount of host cells administered to a mammal (e.g., a human) can be, for example, in the range of one million to 100 billion cells; however, amounts below or above this exemplary range are within the scope of the invention. For example, the daily dose of inventive host cells can be about 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), preferably about 10 million to about 100 billion cells (e.g., about 20 million cells, about million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), more preferably about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells, or a range defined by any two of the foregoing values). In preferred embodiments about 0.5 billion, 1.0 billion, 1.5 billion, 2 billion, 2.5 billion, 3 billion, 3.5 billion. 4 billion, 4.5 billion, 5 billion, 5.5 billion, 6 billion, 6.5 billion, 7 billion, 7.5 billion, 8 billion, 8.5 billion, 9 billion, 9.5 billion or 10 billion cells are administered to the patient in one or more doses.

Therapeutic or prophylactic efficacy can be monitored by periodic assessment of treated patients. For repeated administrations over several days or longer, depending on the condition, the treatment is repeated until a desired suppression of disease symptoms occurs. However, other dosage regimens may be useful and are within the scope of the invention. The desired dosage can be delivered by a single bolus administration of the composition, by multiple bolus administrations of the composition, or by continuous infusion administration of the composition.

As discussed above compositions comprising sensitized the host cells expressing the DLL3 CAR can be administered to a mammal using standard administration techniques, including intravenous, intraperitoneal, subcutaneous, pulmonary, transdermal, intramuscular or intranasal. The composition preferably is suitable for parenteral administration. The term “parenteral,” as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection.

Moreover host cells expressing the DLL3 CAR nucleic acid sequence, or a vector comprising the CAR-encoding nucleic acid sequence, can be administered with one or more additional therapeutic agents, which can be coadministered to the mammal. By “coadministering” is meant administering one or more additional therapeutic agents and the composition comprising the inventive host cells or the inventive vector sufficiently close in time such that the DLL3 CAR can enhance the effect of one or more additional therapeutic agents, or vice versa. In this regard, the composition comprising the sensitized lymphocytes can be administered first, and the one or more additional therapeutic agents can be administered second, or vice versa. Alternatively, the composition comprising the DLL3 sensitized lymphocytes and the one or more additional therapeutic agents can be administered simultaneously.

In selected preferred embodiments the DLL3 sensitized lymphocytes will be administered in conjunction with lymphotoxic therapies to increase the availability of homeostatic cytokines (e.g., IL-7, IL-15, etc.) to support T cell expansion. In such protocols the lymphotoxic therapy will preferably be conducted prior to administration of the sensitized lymphocytes. More specifically it is believed that a lymphodepleting preparative regimen may enhance the efficacy of adoptive cell therapy by reducing endogenous lymphocytes thereby leading to the accumulation of homeostatic cytokines that support expansion and persistence of the administered sensitized lymphocytes. Further, such preparative treatments may lead to a transient reduction in the number and frequency of Tregs thereby diminishing lymphocyte suppression and an induction of gut damage which may lead to the systemic release of bacterial byproducts (e.g., lipopolysaccharides) that activate the innate immune system. Taken together such mechanisms can substantially enhance the receptiveness of the immune environment for the transplanted DLL3 sensitized lymphocytes thereby promoting expansion and persistence of the same.

### VIII. Indications

The present invention preferably provides for the use of DLL3 sensitized lymphocytes for the treatment, maintenance and/or prophylaxis of various disorders including neoplastic, inflammatory, angiogenic and immunologic DLL3 associated disorders. Preferred targets for treatment are neoplastic conditions comprising solid tumors and hematologic malignancies. In certain embodiments the DLL3 CAR treatments of the invention will be used to inhibit, reduce or eliminate tumors or tumorigenic cells expressing DLL3. In selected aspects the disclosed compositions may be used to inhibit tumor cell proliferation. Preferably the “subject” or “patient” to be treated will be human although, as used herein, the terms are expressly held to comprise any mammalian species.

Neoplastic conditions subject to treatment in accordance with the instant invention may be benign or malignant; solid tumors or other blood neoplasia; and may be selected from the group including, but not limited to: adrenal gland tumors, AIDS-associated cancers, alveolar soft part sarcoma, astrocytic tumors, autonomic ganglia tumors, bladder cancer (squamous cell carcinoma and transitional cell carcinoma), blastocoelic disorders, bone cancer (adamantinoma, aneurismal bone cysts, osteochondroma, osteosarcoma), brain and spinal cord cancers, metastatic brain tumors, breast cancer including triple negative breast cancer, carotid body tumors, cervical cancer, chondrosarcoma, chordoma, chromophobe renal cell carcinoma, clear cell carcinoma, colon cancer, colorectal cancer, cutaneous benign fibrous histiocytomas, desmoplastic small round cell tumors, ependymomas, epithelial disorders, Ewing's tumors, extraskeletal myxoid chondrosarcoma, fibrogenesis imperfecta ossium, fibrous dysplasia of the bone, gallbladder and bile duct cancers, gastric cancer, gastrointestinal, gestational trophoblastic disease, germ cell tumors, glandular disorders, head and neck cancers, hypothalamic, intestinal cancer, islet cell tumors, Kaposi's Sarcoma, kidney cancer (nephroblastoma, papillary renal cell carcinoma), leukemias, lipoma/benign lipomatous tumors, liposarcoma/malignant lipomatous tumors, liver cancer (hepatoblastoma, hepatocellular carcinoma), lymphomas, lung cancers (small cell carcinoma, adenocarcinoma, squamous cell carcinoma, large cell carcinoma etc.), macrophagal disorders, medulloblastoma, melanoma, meningiomas, multiple endocrine neoplasia, multiple myeloma, myelodysplastic syndrome, neuroblastoma, neuroendocrine tumors, ovarian cancer, pancreatic cancers, papillary thyroid carcinomas, parathyroid tumors, pediatric cancers, peripheral nerve sheath tumors, phaeochromocytoma, pituitary tumors, prostate cancer, posterious unveal melanoma, rare hematologic disorders, renal metastatic cancer, rhabdoid tumor, rhabdomysarcoma, sarcomas, skin cancer, soft-tissue sarcomas, squamous cell cancer, stomach cancer, stromal disorders, synovial sarcoma, testicular cancer, thymic carcinoma, thymoma, thyroid metastatic cancer, and uterine cancers (carcinoma of the cervix, endometrial carcinoma, and leiomyoma).

In certain embodiments the compounds and compositions of the instant invention will be used as a front line therapy and administered to subjects who have not previously been treated for the cancerous condition. In other embodiments the compounds and compositions of the present invention will be used to treat subjects that have previously been treated (with compositions of the present invention or with other anti-cancer agents) and have relapsed or determined to be refractory to the previous treatment. In selected embodiments the compounds and compositions of the instant invention may be used to treat subjects that have recurrent tumors.

In selected aspects the proliferative disorder will comprise a solid tumor including, but not limited to, adrenal, liver, kidney, bladder, breast, gastric, ovarian, cervical, uterine, esophageal, colorectal, prostate, pancreatic, lung (both small cell and non-small cell), thyroid, carcinomas, sarcomas, glioblastomas and various head and neck tumors.

In other preferred embodiments the compounds or compositions will be administered to a subject suffering from melanoma. More generally the compositions and methods disclosed herein may be used to diagnose, monitor, treat or prevent melanoma. The term “melanoma”, as used herein, includes all types of melanoma including, but not limited to, primary melanoma, malignant melanoma, cutaneous melanoma, extracutaneous melanoma, superficial spreading melanoma, polypoid melanoma, melanocarcinomas, melanoepitheliomas, melanosarcomas, melanoma in situ, nodular malignant melanoma, lentigo maligna melanoma, lentiginous melanoma, lentiginous malignant melanoma, mucosal lentiginous melanoma, mucosal melanoma, acral lentiginous melanoma, soft tissue melanoma, ocular melanoma, invasive melanoma, familial atypical mole and melanoma (FAM-M) syndrome, desmoplastic malignant melanoma or uveal melanoma.

In selected aspects the disclosed DLL3 CAR treatments are especially effective at treating lung cancer, including the following subtypes: small cell lung cancer, non-small cell lung cancer (e.g. squamous cell non-small cell lung cancer or squamous cell small cell lung cancer) and large cell neuroendocrine carcinoma (LCNEC). In selected embodiments the DLL3 sensitive lymphocytes can be administered to patients exhibiting limited stage disease or extensive stage disease. In other preferred embodiments the disclosed conjugated antibodies will be administered to refractory patients (i.e., those whose disease recurs during or shortly after completing a course of initial therapy); sensitive patients (i.e., those whose relapse is longer than 2-3 months after primary therapy); or patients exhibiting resistance to a platinum based agent (e.g. carboplatin, cisplatin, oxaliplatin) and/or a taxane (e.g. docetaxel, paclitaxel, larotaxel or cabazitaxel).

In another particularly preferred embodiment the disclosed DLL3 CAR treatments are effective at treating ovarian cancer, including ovarian-serous carcinoma and ovarian-papillary serous carcinoma.

The disclosed compositions may further be used to prevent, treat or diagnose tumors with neuroendocrine features or phenotypes including neuroendocrine tumors. True or canonical neuroendocrine tumors (NETs) arising from the dispersed endocrine system are relatively rare, with an incidence of 2-5 per 100,000 people, but highly aggressive. Neuroendocrine tumors occur in the kidney, genitourinary tract (bladder, prostate, ovary, cervix, and endometrium), gastrointestinal tract (colon, stomach), thyroid (medullary thyroid cancer), and lung (small cell lung carcinoma and large cell neuroendocrine carcinoma). These tumors may secrete several hormones including serotonin and/or chromogranin A that can cause debilitating symptoms known as carcinoid syndrome. Such tumors can be denoted by positive immunohistochemical markers such as neuron-specific enolase (NSE, also known as gamma enolase, gene symbol=ENO2), CD56 (or NCAM1), chromogranin A (CHGA), and synaptophysin (SYP) or by genes known to exhibit elevated expression such as ASCL1. Unfortunately traditional chemotherapies have not been particularly effective in treating NETs and liver metastasis is a common outcome.

While the disclosed compositions may be advantageously used to treat neuroendocrine tumors they may also be used to treat, prevent or diagnose pseudo neuroendocrine tumors (pNETs) that genotypically or phenotypically mimic, resemble or exhibit common traits with canonical neuroendocrine tumors. Pseudo neuroendocrine tumors or tumors with neuroendocrine features are tumors that arise from cells of the diffuse neuroendocrine system or from cells in which a neuroendocrine differentiation cascade has been aberrantly reactivated during the oncogenic process. Such pNETs commonly share certain phenotypic or biochemical characteristics with traditionally defined neuroendocrine tumors, including the ability to produce subsets of biologically active amines, neurotransmitters, and peptide hormones. Histologically, such tumors (NETs and pNETs) share a common appearance often showing densely connected small cells with minimal cytoplasm of bland cytopathology and round to oval stippled nuclei. For the purposes of the instant invention commonly expressed histological markers or genetic markers that may be used to define neuroendocrine and pseudo neuroendocrine tumors include, but are not limited to, chromogranin A, CD56, synaptophysin, PGP9.5, ASCL1 and neuron-specific enolase (NSE).

Accordingly the sensitized lymphocytes of the instant invention may beneficially be used to treat both pseudo neuroendocrine tumors and canonical neuroendocrine tumors. In this regard the ADCs may be used as described herein to treat neuroendocrine tumors (both NET and pNET) arising in the kidney, genitourinary tract (bladder, prostate, ovary, cervix, and endometrium), gastrointestinal tract (colon, stomach), thyroid (medullary thyroid cancer), and lung (small cell lung carcinoma and large cell neuroendocrine carcinoma). Moreover, the compositions of the instant invention may be used to treat tumors expressing one or more markers selected from the group consisting of NSE, CD56, synaptophysin, chromogranin A, ASCL1 and PGP9.5 (UCHL1). That is, the present invention may be used to treat a subject suffering from a tumor that is NSE⁺ or CD56⁺ or PGP9.5⁺ or ASCL1⁺ or SYP⁺ or CHGA⁺ or some combination thereof.

In another preferred embodiment the DLL3 CAR treatments of the instant invention may be used in maintenance therapy to reduce or eliminate the chance of tumor recurrence following the initial presentation of the disease. Preferably the disorder will have been treated and the initial tumor mass eliminated, reduced or otherwise ameliorated so the patient is asymptomatic or in remission. At such time the subject may be administered pharmaceutically effective amounts of the disclosed DLL3 CAR treatments one or more times even though there is little or no indication of disease using standard diagnostic procedures. In some embodiments, the modulators will be administered on a regular schedule over a period of time, such as weekly, every two weeks, monthly, every six weeks, every two months, every three months every six months or annually. Given the teachings herein, one skilled in the art could readily determine favorable dosages and dosing regimens to reduce the potential of disease recurrence. Moreover such treatments could be continued for a period of weeks, months, years or even indefinitely depending on the patient response and clinical and diagnostic parameters.

In yet another preferred embodiment the DLL3 CAR treatments of the present invention may be used to prophylactically or as an adjuvant therapy to prevent or reduce the possibility of tumor metastasis following a debulking procedure. As used in the instant disclosure a “debulking procedure” is defined broadly and shall mean any procedure, technique or method that eliminates, reduces, treats or ameliorates a tumor or tumor proliferation. Exemplary debulking procedures include, but are not limited to, surgery, radiation treatments (i.e., beam radiation), chemotherapy, immunotherapy or ablation. At appropriate times readily determined by one skilled in the art in view of the instant disclosure the disclosed DLL3 CAR treatments may be administered as suggested by clinical, diagnostic or theragnostic procedures to reduce tumor metastasis. The DLL3 sensitized lymphocytes may be administered one or more times at pharmaceutically effective dosages as determined using standard techniques. Preferably the dosing regimen will be accompanied by appropriate diagnostic or monitoring techniques that allow it to be modified.

Yet other embodiments of the invention comprise administering the disclosed DLL3 CAR treatments to subjects that are asymptomatic but at risk of developing a proliferative disorder. That is, the DLL3 CAR treatments of the instant invention may be used in a truly preventative sense and given to patients that have been examined or tested and have one or more noted risk factors (e.g., genomic indications, family history, in vivo or in vitro test results, etc.) but have not developed neoplasia. In such cases those skilled in the art would be able to determine an effective dosing regimen through empirical observation or through accepted clinical practices.

### IX. Combination Therapies

As previously discussed it will be appreciated that the DLL3 CAR treatments described herein may be used in combination with other clinical oncology treatments. In general the treatments of the instant invention may be used with a therapeutic moiety or a drug such as an anti-cancer agent including, but not limited to, cytotoxic agents, cytostatic agents, anti-angiogenic agents, debulking agents, chemotherapeutic agents, radiotherapeutic agents, targeted anti-cancer agents, biological response modifiers, cancer vaccines, cytokines, hormone therapies, anti-metastatic agents and immunotherapeutic agents.

Combination therapies may be useful in preventing or treating cancer and in preventing metastasis or recurrence of cancer. “Combination therapy”, as used herein, means the administration of a combination comprising at least one DLL3 CAR treatment and at least one therapeutic moiety (e.g., anti-cancer agent) wherein the combination preferably has therapeutic synergy or improves the measurable therapeutic effects in the treatment of cancer over (i) the DLL3 CAR treatment used alone, or (ii) the therapeutic moiety used alone, or (iii) the use of the therapeutic moiety in combination with another therapeutic moiety without the addition of DLL3 CAR treatment. The terms “therapeutic synergy” or “synergy”, as used herein, means the combination of an DLL3 CAR treatment and one or more therapeutic moiety(ies) having a therapeutic effect greater than the additive effect of the combination of the DLL3 CAR treatment and the one or more therapeutic moiety(ies).

Desired outcomes of the disclosed combinations are quantified by comparison to a control or baseline measurement. As used herein, relative terms such as “improve,” “increase,” or “reduce” indicate values relative to a control, such as a measurement in the same individual prior to initiation of treatment described herein, or a measurement in a control individual (or multiple control individuals) in the absence of DLL3 CAR treatments described herein but in the presence of other therapeutic moiety(ies) such as standard of care treatment. A representative control individual is an individual afflicted with the same form of cancer as the individual being treated, who is about the same age as the individual being treated (to ensure that the stages of the disease in the treated individual and the control individual are comparable.)

Changes or improvements in response to therapy are generally statistically significant. As used herein, the term “significance” or “significant” relates to a statistical analysis of the probability that there is a non-random association between two or more entities. To determine whether or not a relationship is “significant” or has “significance,” a “p-value” can be calculated. P-values that fall below a user-defined cut-off point are regarded as significant. A p-value less than or equal to 0.1, less than 0.05, less than 0.01, less than 0.005, or less than 0.001 may be regarded as significant.

A synergistic therapeutic effect may be an effect of at least about two-fold greater than the therapeutic effect elicited by a single therapeutic moiety or DLL3 CAR treatment, or the sum of the therapeutic effects elicited by the DLL3 CAR treatment or the single therapeutic moiety(ies) of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the therapeutic effect elicited by a single therapeutic moiety or DLL3 CAR treatment or the sum of the therapeutic effects elicited by the DLL3 CAR treatment or the single therapeutic moiety(ies) of a given combination, or at least 20%, or at least 30%, or at least 40%, or at least 50%, or at least 60%, or at least 70%, or at least 80%, or at least 90%, or at least 100%, or more. A synergistic effect is also an effect that permits reduced dosing of therapeutic agents when they are used in combination.

In practicing combination therapy, the DLL3 CAR treatment and therapeutic moiety(ies) may be administered to the subject simultaneously, either in a single composition, or as two or more distinct compositions using the same or different administration routes. Alternatively, treatment with the DLL3 CAR treatment may precede or follow the therapeutic moiety treatment by, e.g., intervals ranging from minutes to weeks. In one embodiment, both the CAR therapeutic moiety and the antibody or ADC are administered within about 5 minutes to about two weeks of each other. In yet other embodiments, several days (2, 3, 4, 5, 6 or 7), several weeks (1, 2, 3, 4, 5, 6, 7 or 8) or several months (1, 2, 3, 4, 5, 6, 7 or 8) may lapse between administration of the antibody and the therapeutic moiety.

The combination therapy can be administered until the condition is treated, palliated or cured on various schedules such as once, twice or three times daily, once every two days, once every three days, once weekly, once every two weeks, once every month, once every two months, once every three months, once every six months, or may be administered continuously. The antibody and therapeutic moiety(ies) may be administered on alternate days or weeks; or a sequence of DLL3 CAR treatments may be given, followed by one or more treatments with the additional therapeutic moiety. In one embodiment an DLL3 CAR is administered in combination with one or more therapeutic moiety(ies) for short treatment cycles. In other embodiments the combination treatment is administered for long treatment cycles. The combination therapy can be administered via any route.

In selected embodiments the compounds and compositions of the present invention may be used in conjunction with checkpoint inhibitors such as PD-1 inhibitors or PD-L1 inhibitors. PD-1, together with its ligand PD-L1, are negative regulators of the antitumor T lymphocyte response. In one embodiment the combination therapy may comprise the administration of DLL3 sensitized lymphocytes together with an anti-PD-1 antibody (e.g. pembrolizumab, nivolumab, pidilizumab) and optionally one or more other therapeutic moiety(ies). In another embodiment the combination therapy may comprise the administration of DLL3 sensitized lymphocytes together with an anti-PD-L1 antibody (e.g. avelumab, atezolizumab, durvalumab) and optionally one or more other therapeutic moiety(ies). In yet another embodiment, the combination therapy may comprise the administration of DLL3 sensitized lymphocytes together with an anti PD-1 antibody or anti-PD-L1 administered to patients who continue progress following treatments with checkpoint inhibitors and/or targeted BRAF combination therapies (e.g., ipilimumab and vemurafenib or dabrafinib).

In some embodiments the sensitized lymphocytes may be used in combination with various first line cancer treatments. In one embodiment the combination therapy comprises the use of the compositions of the instant invention and a cytotoxic agent such as ifosfamide, mytomycin C, vindesine, vinblastine, etoposide, ironitecan, gemcitabine, taxanes, vinorelbine, methotrexate, and pemetrexed) and optionally one or more other therapeutic moiety(ies).

In another embodiment the combination therapy comprises the use of an DLL3 CAR treatment and a platinum-based drug (e.g. carboplatin or cisplatin) and optionally one or more other therapeutic moiety(ies) (e.g. vinorelbine; gemcitabine; a taxane such as, for example, docetaxel or paclitaxel; irinotican; or pemetrexed).

In one embodiment, for example, in the treatment of BR-ERPR, BR-ER or BR-PR cancer, the combination therapy comprises the use of an DLL3 CAR treatment and one or more therapeutic moieties described as “hormone therapy”. “Hormone therapy” as used herein, refers to, e.g., tamoxifen; gonadotropin or luteinizing releasing hormone (GnRH or LHRH); everolimus and exemestane; toremifene; or aromatase inhibitors (e.g. anastrozole, letrozole, exemestane or fulvestrant).

In another embodiment, for example, in the treatment of BR-HER2, the combination therapy comprises the use of an DLL3 CAR treatment and trastuzumab or ado-trastuzumab emtansine and optionally one or more other therapeutic moiety(ies) (e.g. pertuzumab and/or docetaxel).

In some embodiments, for example, in the treatment of metastatic breast cancer, the combination therapy comprises the use of an DLL3 CAR treatment and a taxane (e.g. docetaxel or paclitaxel) and optionally an additional therapeutic moiety(ies), for example, an anthracycline (e.g. doxorubicin or epirubicin) and/or eribulin.

In another embodiment, for example, in the treatment of metastatic or recurrent breast cancer or BRCA-mutant breast cancer, the combination therapy comprises the use of an DLL3 CAR treatment and megestrol and optionally an additional therapeutic moiety(ies).

In further embodiments, for example, in the treatment of BR-TNBC, the combination therapy comprises the use of an DLL3 CAR treatment and a poly ADP ribose polymerase (PARP) inhibitor (e.g. BMN-673, olaparib, rucaparib and veliparib) and optionally an additional therapeutic moiety(ies).

In another embodiment, for example, in the treatment of breast cancer, the combination therapy comprises the use of an DLL3 CAR treatment and cyclophosphamide and optionally an additional therapeutic moiety(ies) (e.g. doxorubicin, a taxane, epirubicin, 5-FU and/or methotrexate.

In another embodiment combination therapy for the treatment of EGFR-positive NSCLC comprises the use of an DLL3 CAR treatment and afatinib and optionally one or more other therapeutic moiety(ies) (e.g. erlotinib and/or bevacizumab).

In another embodiment combination therapy for the treatment of EGFR-positive NSCLC comprises the use of an DLL3 CAR treatment and erlotinib and optionally one or more other therapeutic moiety(ies) (e.g. bevacizumab).

In another embodiment combination therapy for the treatment of ALK-positive NSCLC comprises the use of an DLL3 CAR treatment and ceritinib and optionally one or more other therapeutic moiety(ies).

In another embodiment combination therapy for the treatment of ALK-positive NSCLC comprises the use of an DLL3 CAR treatment and crizotinib and optionally one or more other therapeutic moiety(ies).

In another embodiment the combination therapy comprises the use of an DLL3 CAR treatment and bevacizumab and optionally one or more other therapeutic moiety(ies) (e.g. a taxane such as, for example, docetaxel or paclitaxel; and/or a platinum analog).

In another embodiment the combination therapy comprises the use of an DLL3 CAR treatment and bevacizumab and optionally one or more other therapeutic moiety(ies) (e.g. gemcitabine and/or a platinum analog).

In one embodiment the combination therapy comprises the use of an DLL3 CAR treatment and a platinum-based drug (e.g. carboplatin or cisplatin) analog and optionally one or more other therapeutic moiety(ies) (e.g. a taxane such as, for example, docetaxel and paclitaxel).

In one embodiment the combination therapy comprises the use of an DLL3 CAR treatment and platinum-based drug (e.g. carboplatin or cisplatin) analog and optionally one or more other therapeutic moiety(ies) (e.g. a taxane such, for example, docetaxel and paclitaxel and/or gemcitabine and/or doxorubicin).

In a particular embodiment the combination therapy for the treatment of platinum-resistant tumors comprises the use of a DLL3 CAR treatment and doxorubicin and/or etoposide and/or gemcitabine and/or vinorelbine and/or ifosfamide and/or leucovorin-modulated 5-fluoroucil and/or bevacizumab and/or tamoxifen; and optionally one or more other therapeutic moiety(ies).

In another embodiment the combination therapy comprises the use of a DLL3 CAR treatment and a PARP inhibitor and optionally one or more other therapeutic moiety(ies).

In another embodiment the combination therapy comprises the use of an DLL3 CAR treatment and bevacizumab and optionally cyclophosphamide.

The combination therapy may comprise a DLL3 CAR treatment and a chemotherapeutic moiety that is effective on a tumor comprising a mutated or aberrantly expressed gene or protein (e.g. BRCA1).

More generally the DLL3 CAR treatments of the instant invention may be used in combination with a number of anti-cancer agents. The term “anti-cancer agent” or “chemotherapeutic agent” as used herein is one subset of “therapeutic moieties”, which in turn is a subset of the agents described as “pharmaceutically active moieties”. More particularly “anti-cancer agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, biological response modifiers, therapeutic antibodies, cancer vaccines, cytokines, hormone therapy, anti-metastatic agents and immunotherapeutic agents. It will be appreciated that in selected embodiments as discussed above, such anti-cancer agents may comprise antibody drug conjugates and may be associated with antibodies prior to administration.

The term “cytotoxic agent”, which can also be an anti-cancer agent means a substance that is toxic to the cells and decreases or inhibits the function of cells and/or causes destruction of cells. Typically, the substance is a naturally occurring molecule derived from a living organism (or a synthetically prepared natural product). Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, saponaria officinalis inhibitor, mitegellin, restrictocin, phenomycin, neomycin, and the tricothecenes) or animals, (e.g., cytotoxic RNases, such as extracellular pancreatic RNases; DNase I, including fragments and/or variants thereof).

An anti-cancer agent can include any chemical agent that inhibits, or is designed to inhibit, a cancerous cell or a cell likely to become cancerous or generate tumorigenic progeny (e.g., tumorigenic cells). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. Such agents are often administered, and are often most effective, in combination, e.g., in the formulation CHOP.

Examples of anti-cancer agents that may be used in combination with DLL3 CAR treatment of the invention include, but are not limited to, alkylating agents, alkyl sulfonates, anastrozole, amanitins, aziridines, ethylenimines and methylamelamines, acetogenins, a camptothecin, BEZ-235, bortezomib, bryostatin, callystatin, CC-1065, ceritinib, crizotinib, cryptophycins, dolastatin, duocarmycin, eleutherobin, erlotinib, pancratistatin, a sarcodictyin, spongistatin, nitrogen mustards, antibiotics, enediyne dynemicin, bisphosphonates, esperamicin, chromoprotein enediyne antiobiotic chromophores, aclacinomysins, actinomycin, authramycin, azaserine, bleomycins, cactinomycin, canfosfamide, carabicin, carminomycin, carzinophilin, chromomycinis, cyclosphosphamide, dactinomycin, daunorubicin, detorubicin, 6-diazo-5-oxo-L-norleucine, doxorubicin, epirubicin, esorubicin, exemestane, fluorouracil, fulvestrant, gefitinib, idarubicin, lapatinib, letrozole, lonafamib, marcellomycin, megestrol acetate, mitomycins, mycophenolic acid, nogalamycin, olivomycins, pazopanib, peplomycin, potfiromycin, puromycin, quelamycin, rapamycin, rodorubicin, sorafenib, streptonigrin, streptozocin, tamoxifen, tamoxifen citrate, temozolomide, tepodina, tipifarnib, tubercidin, ubenimex, vandetanib, vorozole, XL-147, zinostatin, zorubicin; anti-metabolites, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, polysaccharide complex, razoxane; rhizoxin; SF-1126, sizofiran; spirogermanium; tenuazonic acid; triaziquone; 2,2′,2″-trichlorotriethylamine; trichothecenes (T-2 toxin, verracurin A, roridin A and anguidine); urethan; vindesine; dacarbazine; mannomustine; mitobronitol; mitolactol; pipobroman; gacytosine; arabinoside; cyclophosphamide; thiotepa; taxoids, chloranbucil; gemcitabine; 6-thioguanine; mercaptopurine; methotrexate; platinum analogs, vinbiastine; platinum; etoposide; ifosfamide; mitoxantrone; vincristine; vinorelbine; novantrone; teniposide; edatrexate; daunomycin; aminopterin; xeloda; ibandronate; irinotecan, topoisomerase inhibitor RFS 2000; difluorometlhylornithine; retinoids; capecitabine; combretastatin; leucovorin; oxaliplatin; XL518, inhibitors of PKC-alpha, Raf, H-Ras, EGFR and VEGF-A that reduce cell proliferation and pharmaceutically acceptable salts or solvates, acids or derivatives of any of the above. Also included in this definition are anti-hormonal agents that act to regulate or inhibit hormone action on tumors such as anti-estrogens and selective estrogen receptor antibodies, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts or solvates, acids or derivatives of any of the above.

Particularly preferred anti-cancer agents comprise commercially or clinically available compounds such as erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®, and doxorubicin (ADRIAMYCIN®). Additional commercially or clinically available anti-cancer agents comprise oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafamib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifamib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rlNN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); vinorelbine (NAVELBINE®); capecitabine (XELODA®, Roche), tamoxifen (including NOLVADEX®; tamoxifen citrate, FARESTON® (toremifine citrate) MEGASES (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca).

The term “pharmaceutically acceptable salt” or “salt” means organic or inorganic salts of a molecule or macromolecule. Acid addition salts can be formed with amino groups. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′ methylene bis-(2-hydroxy 3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Where multiple charged atoms are part of the pharmaceutically acceptable salt, the salt can have multiple counter ions. Hence, a pharmaceutically acceptable salt can have one or more charged atoms and/or one or more counterion.

“Pharmaceutically acceptable solvate” or “solvate” refers to an association of one or more solvent molecules and a molecule or macromolecule. Examples of solvents that form pharmaceutically acceptable solvates include, but are not limited to, water, isopropanol, ethanol, methanol, DMSO, ethyl acetate, acetic acid, and ethanolamine.

In other embodiments the DLL3 CAR treatments of the instant invention may be used in combination with any one of a number of antibodies (or immunotherapeutic agents) presently in clinical trials or commercially available. The disclosed antibodies may be used in combination with an antibody selected from the group consisting of abagovomab, adecatumumab, afutuzumab, alemtuzumab, altumomab, amatuximab, anatumomab, arcitumomab, atezolizumab, avelumab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, durvalumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nivolumab, nofetumomabn, obinutuzumab, ocaratuzumab, ofatumumab, olaratumab, olaparib, onartuzumab, oportuzumab, oregovomab, panitumumab, parsatuzumab, patritumab, pembrolizumab, pemtumomab, pertuzumab, pidilizumab, pintumomab, pritumumab, racotumomab, radretumab, ramucirumab, rilotumumab, rituximab, robatumumab, satumomab, selumetinib, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, 3F8, MDX-1105 and combinations thereof.

Other particularly preferred embodiments comprise the use of the disclosed compositions with antibodies approved for cancer therapy including, but not limited to, rituximab, gemtuzumab ozogamcin, alemtuzumab, ibritumomab tiuxetan, tositumomab, bevacizumab, cetuximab, patitumumab, ofatumumab, ipilimumab and brentuximab vedotin. Those skilled in the art will be able to readily identify additional anti-cancer agents that are compatible with the teachings herein.

The present invention also provides for the combination of the DLL3 CAR treatments with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed DLL3 CAR treatments may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

### X. Diagnostics

The invention provides in vitro and in vivo methods for detecting, diagnosing or monitoring the efficiency of any lymphocyte transduction or the effect of any DLL3 sensitized lymphocytes on tumor cells including tumorigenic cells. Such methods include identifying an individual having cancer (e.g., a DLL3 positive tumor) for treatment or monitoring progression of a cancer, comprising interrogating the patient or a sample obtained from a patient (either in vivo or in vitro) with an antibody as described herein before during or after treatment with DLL3 sensitized lymphocytes and detecting presence or absence, or level of association, of the antibody to bound or free target molecules in the sample. In some embodiments the DLL3 antibody will comprise a detectable label or reporter molecule as described herein. In yet other embodiments (e.g., In situ hybridization or ISH) a nucleic acid probe that reacts with a genomic DLL3 determinant will be used in the detection, diagnosis or monitoring of the proliferative disorder.

More generally the presence and/or levels of DLL3 determinants may be measured using any of a number of techniques available to the person of ordinary skill in the art for protein or nucleic acid analysis, e.g., direct physical measurements (e.g., mass spectrometry), binding assays (e.g., immunoassays, agglutination assays, and immunochromatographic assays), Polymerase Chain Reaction (PCR, RT-PCR; RT-qPCR) technology, branched oligonucleotide technology, Northern blot technology, oligonucleotide hybridization technology and in situ hybridization technology. The method may also comprise measuring a signal that results from a chemical reaction, e.g., a change in optical absorbance, a change in fluorescence, the generation of chemiluminescence or electrochemiluminescence, a change in reflectivity, refractive index or light scattering, the accumulation or release of detectable labels from the surface, the oxidation or reduction or redox species, an electrical current or potential, changes in magnetic fields, etc. Suitable detection techniques may detect binding events by measuring the participation of labeled binding reagents through the measurement of the labels via their photoluminescence (e.g., via measurement of fluorescence, time-resolved fluorescence, evanescent wave fluorescence, up-converting phosphors, multi-photon fluorescence, etc.), chemiluminescence, electrochemiluminescence, light scattering, optical absorbance, radioactivity, magnetic fields, enzymatic activity (e.g., by measuring enzyme activity through enzymatic reactions that cause changes in optical absorbance or fluorescence or cause the emission of chemiluminescence). Alternatively, detection techniques may be used that do not require the use of labels, e.g., techniques based on measuring mass (e.g., surface acoustic wave measurements), refractive index (e.g., surface plasmon resonance measurements), or the inherent luminescence of an analyte.

In some embodiments, the association of the detection agent with particular cells or cellular components in the sample indicates that the sample may contain tumorigenic cells, thereby denoting that the individual having cancer may be effectively treated with the compositions as described herein.

In certain preferred embodiments the assays may comprise immunohistochemistry (IHC) assays or variants thereof (e.g., fluorescent, chromogenic, standard ABC, standard LSAB, etc.), immunocytochemistry or variants thereof (e.g., direct, indirect, fluorescent, chromogenic, etc.) or In situ hybridization (ISH) or variants thereof (e.g., chromogenic in situ hybridization (CISH) or fluorescence in situ hybridization (DNA-FISH or RNA-FISH]))

In this regard certain aspects of the instant invention comprise the use of labeled DLL3 for immunohistochemistry (IHC). More particularly DLL3 IHC may be used as a diagnostic tool to aid in the diagnosis of various proliferative disorders and to monitor the potential response to treatments including DLL3 antibody therapy. As discussed herein and shown in the Examples below compatible diagnostic assays may be performed on tissues that have been chemically fixed (including but not limited to: formaldehyde, gluteraldehyde, osmium tetroxide, potassium dichromate, acetic acid, alcohols, zinc salts, mercuric chloride, chromium tetroxide and picric acid) and embedded (including but not limited to: glycol methacrylate, paraffin and resins) or preserved via freezing. Such assays can be used to guide treatment decisions and determine dosing regimens and timing.

Other particularly compatible aspects of the invention involve the use of in situ hybridization to detect or monitor DLL3 determinants. In situ hybridization technology or ISH is well known to those of skill in the art. Briefly, cells are fixed and detectable probes which contain a specific nucleotide sequence are added to the fixed cells. If the cells contain complementary nucleotide sequences, the probes, which can be detected, will hybridize to them. Using the sequence information set forth herein, probes can be designed to identify cells that express genotypic DLL3 determinants. Probes preferably hybridize to a nucleotide sequence that corresponds to such determinants. Hybridization conditions can be routinely optimized to minimize background signal by non-fully complementary hybridization though preferably the probes are preferably fully complementary to the selected DLL3 determinant. In selected embodiments the probes are labeled with fluorescent dye attached to the probes that is readily detectable by standard fluorescent methodology.

Compatible in vivo theragnostics or diagnostic assays may comprise art-recognized imaging or monitoring techniques such as magnetic resonance imaging, computerized tomography (e.g. CAT scan), positron tomography (e.g., PET scan) radiography, ultrasound, etc., as would be known by those skilled in the art.

In a particularly preferred embodiment the antibodies disclosed herein may be used to detect and quantify levels of a particular determinant (e.g., DLL3) in a patient sample (e.g., plasma or blood) which may, in turn, be used to detect, diagnose or monitor proliferative disorders both before and after treatment with the DLL3 sensitized lymphocytes. In related embodiments the antibodies disclosed herein may be used to detect, monitor and/or quantify circulating tumor cells either in vivo or in vitro (WO 2012/0128801) in combination with the disclosed treatments by DLL3 sensitized lymphocytes. In still other embodiments the circulating tumor cells may comprise tumorigenic cells.

In certain embodiments of the invention, the tumorigenic cells in a subject or a sample from a subject may be assessed or characterized using the disclosed antibodies prior to DLL3 CAR therapy or regimen to establish a baseline. In other examples, the tumorigenic cells can be assessed from a sample that is derived from a subject that was treated.

### XI. Articles of Manufacture

The invention further includes pharmaceutical packs and kits comprising one or more containers or receptacles, wherein a container can comprise one or more transformation doses of a DLL3 CAR plasmid or vector of the invention. In certain embodiments, the pack or kit contains a vector preparation (e.g., lentiviral or retroviral) comprising a nucleic acid encoding a DLL3 CAR, with or without one or more additional reagents and optionally a means of effecting transduction. Preferably the kit will further include the means to monitor and characterize the preparation of DLL3 sensitized lymphocytes prior to administration.

Certain other embodiments will comprise a container or receptacle incorporating, containing or holding a liquid formulation (dispersion, suspension or solution) of DLL3 sensitized lymphocytes. In selected embodiments the DLL3 sensitized lymphocytes will be allogenic. In other embodiments the DLL3 sensitized lymphocytes will comprise autologous host cells. In certain other embodiments the liquid formulation will comprise a pharmaceutically acceptable carrier.

In selected aspects kits compatible with the invention would allow a user to produce the DLL3 sensitive lymphocytes, monitor transduction rates and characterize the resulting DLL3 sensitive lymphocyte population to ensure quality prior to administration. Accordingly, a kit of the invention may generally contain a pharmaceutically acceptable formulation of the CAR nucleic acid (or vector) and, optionally, one or more reagents in the same or different containers. In preferred embodiments the DLL3 CAR vectors will comprise viral vectors (e.g., lentiviral or retroviral) that allow for transduction of selected host cells to provide the disclosed sensitized lymphocytes. In certain embodiments the selected host cell will be autologous while in other embodiments the selected host cells will be allogeneic. Some aspects of the invention are directed to kits including allogeneic cells along with the DLL3 CAR vector. Yet other embodiments comprise kits or containers or receptacles incorporating a pharmaceutical composition comprising allogeneic DLL3 sensitized lymphocytes. Still other articles of manufacture comprise a container incorporating or holding a liquid formulation of autologous DLL3 sensitized lymphocytes in a pharmaceutically acceptable carrier. In all such kits the container may comprise an infusion bag, vial, syringe or bottle that would allow the DLL3 sensitized lymphocytes to be directly administered to the patient.

The kits may also contain other pharmaceutically acceptable formulations or devices, either for diagnosis or combination therapy. Examples of diagnostic devices or instruments include those that can be used to detect, monitor, quantify or profile cells or markers associated with the DLL3 sensitive lymphocytes, transformation efficiency or the proliferative disorder to be treated. In particularly preferred embodiments the devices may be used to detect, monitor and/or quantify circulating tumor cells either in vivo or in vitro. In still other preferred embodiments the circulating tumor cells may comprise tumorigenic cells.

When selected components of the kit (e.g., DLL3 sensitized lymphocytes) are provided in one or more liquid solutions, the liquid solution can be non-aqueous though an aqueous solution is preferred, with a sterile aqueous solution being particularly preferred. The formulations of the kit (e.g., a viral vector) can also be provided as dried powder(s) or in lyophilized form that can be reconstituted upon addition of an appropriate liquid. The liquid used for reconstitution can be contained in a separate container. Such liquids can comprise sterile, pharmaceutically acceptable buffer(s) or other diluent(s) such as bacteriostatic water for injection, phosphate-buffered saline, Ringer's solution or dextrose solution. Where the kit comprises the CAR plasmid or vectors of the invention in combination with additional reagents, the solution may be pre-mixed, either in a molar equivalent combination, or with one component in excess of the other. Alternatively, the plasmids of the invention and any optional co-reagents can be maintained separately within distinct containers prior to transformation of the lymphocytes. In other preferred embodiments container(s) of the kit may comprise liquid formulations of allogeneic DLL3 sensitized lymphocytes.

The disclosed kits can comprise one or multiple containers and a label or package insert in, on or associated with the container(s), indicating that the enclosed composition is useful for treating a proliferative disorder or for preparing cells for treating the selected disease. Suitable containers or receptacles include, for example, bottles, vials, syringes, etc. The containers can be formed from a variety of materials such as glass or plastic. The container(s) can comprise a sterile access port, for example, the container may be an intravenous solution bag or a vial having a stopper that can be pierced by a hypodermic injection needle.

In some embodiments the kit can contain a means by which to administer the DLL3 sensitized lymphocytes and any optional components to a patient, e.g., one or more needles or syringes (pre-filled or empty), an eye dropper, pipette, or other such like apparatus, from which the formulation may be injected or introduced into the subject or applied to a diseased area of the body. The kits of the invention will also typically include a means for containing the vials, or such like, and other components in close confinement for commercial sale, such as, e.g., blow-molded plastic containers into which the desired vials and other apparatus are placed and retained method.

### XII. Miscellaneous

Unless otherwise defined herein, scientific and technical terms used in connection with the invention shall have the meanings that are commonly understood by those of ordinary skill in the art. Further, unless otherwise required by context, singular terms shall include pluralities and plural terms shall include the singular. In addition, ranges provided in the specification and appended claims include both end points and all points between the end points. Therefore, a range of 2.0 to 3.0 includes 2.0, 3.0, and all points between 2.0 and 3.0.

Generally, techniques of cell and tissue culture, molecular biology, immunology, microbiology, genetics and chemistry described herein are those well known and commonly used in the art. The nomenclature used herein, in association with such techniques, is also commonly used in the art. The methods and techniques of the invention are generally performed according to conventional methods well known in the art and as described in various references that are cited throughout the present specification unless otherwise indicated.

### XIII. References

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference, regardless of whether the phrase “incorporated by reference” is or is not used in relation to the particular reference. The foregoing detailed description and the examples that follow have been given for clarity of understanding only. No unnecessary limitations are to be understood therefrom. The invention is not limited to the exact details shown and described. Variations obvious to one skilled in the art are included in the invention defined by the claims. Any section headings used herein are for organizational purposes only and are not to be construed as limiting the subject matter described method.

### XIV. Sequences

Appended to the instant application are figures and a sequence listing comprising a number of nucleic acid and amino acid sequences. The following Table 2 provides a summary of the included sequences.

TABLE 2

| SEQ ID NO: | Description |
|---|---|
| 1 | DLL3 isoform 1 protein |
| 2 | DLL3 isoform 2 protein |
| 3 | Epitope protein-SC16.23 |
| 4 | Epitope protein-SC16.34 & SC 16.56 |
| 5 | Kappa constant region protein |
| 6 | IgG1 constant region protein |
| 7 | (G45)3 pentamer |
| 8 | h16.15 scFv protein |
| 9 | SCT1-h16.15 CAR DNA |
| 10 | SCT1-h16.15 CAR protein |
| 11 | h16.13 scFv DNA |
| 12 | h16.13 scFv protein |
| 13 | h16.25 scFv DNA |
| 14 | h16.25 scFv protein |
| 15 | h16.15 scFv DNA |
| 16 | SCT1-h16.13 CAR DNA |
| 17 | SCT1-h16.13 CAR protein |
| 18 | SCT1-h16.25 CAR DNA |
| 19 | SCT1-h16.25 CAR protein |
| 20 | 5016.3 VL DNA (aligned with encoded protein) |
| 21 | 5016.3 VL protein |
| 22 | 5016.3 VH DNA (aligned with encoded protein) |
| 23 | 5016.3 VH protein |
| 24-387 | Additional murine clones as in SEQ ID NOs: 20-23 |
| 388-407 | Humanized clones as in SEQ ID NOs: 20-23 |
| 408, 409, 410 | hSC16.13 CDRL1, CDRL2, CDRL3 |
| 411, 412, 413 | hSC16.13 CDRH1, CDRH2, CDRH3 |
| 414, 415, 416 | hSC16.15 CDRL1, CDRL2, CDRL3 |
| 417, 418, 419 | hSC16.15 CDRH1, CDRH2, CDRH3 |
| 420, 421, 422 | hSC16.25 CDRL1, CDRL2, CDRL3 |
| 423, 424, 425 | hSC16.25 CDRH1, CDRH2, CDRH3 |
| 426, 427, 428 | hSC16.34 CDRL1, CDRL2, CDRL3 |
| 429, 430, 431 | hSC16.34 CDRH1, CDRH2, CDRH3 |
| 432, 433, 434 | hSC16.56 CDRL1, CDRL2, CDRL3 |
| 435, 436, 437 | hSC16.56 CDRH1, CDRH2, CDRH3 |

As discussed in Example 2 below, Table 2 above may further be used to designate SEQ ID NOS for exemplary Kabat CDRs delineated in FIGS. 1A and 1B. More particularly FIGS. 1A and 1B denote the three Kabat CDRs of each heavy (CDRH) and light (CDRL) chain variable region sequence and Table 2 above provides for assignment of a SEQ ID designation that may be applied to each CDRL1, CDRL2 and CDRL3 of the light chain and each CDRH1, CDRH2 and CDRH3 of the heavy chain. Using this methodology each unique CDR set forth in FIGS. 1A and 1B may be assigned a sequential SEQ ID NO and can be used to provide the derived antibodies of the instant invention.

### XV. Tumor Listing

PDX tumor cell types are denoted by an abbreviation followed by a number, which indicates the particular tumor cell line. The passage number of the tested sample is indicated by p0-p# appended to the sample designation where p0 is indicative of an unpassaged sample obtained directly from a patient tumor and p# is indicative of the number of times the tumor has been passaged through a mouse prior to testing. As used herein, the abbreviations of the tumor types and subtypes are shown in Table 3 as follows:

TABLE 3

| Tumor Type | Abbreviation | Tumor subtype | Abbreviation |
|---|---|---|---|
| Breast | BR | | |
| | | estrogen receptor positive and/or | BR-ERPR |
| | | progesterone receptor positive | |
| | | ERBB2/Neu positive | BR-ERBB2/Neu |
| | | HER2 positive | BR-HER2 |
| | | triple-negative | TNBC |
| | | claudin subtype of | TNBC-CLDN |
| | | triple-negative | |
| colorectal | CR | | |
| endometrial | EN | | |
| gastric | GA | | |
| | | diffuse adenocarcinoma | GA-Ad-Dif/Muc |
| | | intestinal adenocarcinoma | GA-Ad-Int |
| | | stromal tumors | GA-GIST |
| glioblastoma | GB | | |
| head and neck | HN | | |
| kidney | KDY | | |
| | | clear renal cell carcinoma | KDY-CC |
| | | papillary renal cell carcinoma | KDY-PAP |
| | | transitional cell or urothelial | KDY-URO |
| | | carcinoma | |
| | | unknown | KDY-UNK |
| liver | LIV | | |
| | | hepatocellular carcinoma | LIV-HCC |
| | | cholangiocarcinoma | LIV-CHOL |
| lymphoma | LN | | |
| lung | LU | | |
| | | adenocarcinoma | LU-Ad |
| | | carcinoid | LU-CAR |
| | | large cell neuroendocrine | LU-LCC |
| | | non-small cell | NSCLC |
| | | squamous cell | LU-SCC |
| | | small cell | SCLC |
| | | spindle cell | LU-SPC |
| melanoma | MEL | | |
| ovarian | OV | | |
| | | clear cell | OV-CC |
| | | endometroid | OV-END |
| | | mixed subtype | OV-MIX |
| | | malignant mixed mesodermal | OV-MMMT |
| | | mucinous | OV-MUC |
| | | neuroendocrine | OV-NET |
| | | papillary serous | OV-PS |
| | | serous | OV-S |
| | | small cell | OV-SC |
| | | transitional cell carcinoma | OV-TCC |
| pancreatic | PA | | |
| | | acinar cell carcinoma | PA-ACC |
| | | duodenal carcinoma | PA-DC |
| | | mucinous adenocarcinoma | PA-MAD |
| | | neuroendocrine | PA-NET |
| | | adenocarcinoma | PA-PAC |
| | | adenocarcinoma exocrine type | PA-PACe |
| | | ductal adenocarcinoma | PA-PDAC |
| | | ampullary adenocarcinoma | PA-AAC |
| prostate | PR | | |
| skin | SK | | |
| | | melanoma | MEL |
| | | squamous cell carcinomas | SK-SCC |

### EXAMPLES

The invention, thus generally described above, will be understood more readily by reference to the following examples, which are provided by way of illustration and are not intended to be limiting of the instant invention. The examples are not intended to represent that the experiments below are all or the only experiments performed. Unless indicated otherwise, parts are parts by weight, molecular weight is weight average molecular weight, temperature is in degrees Centigrade, and pressure is at or near atmospheric.

### Example 1

### Generation of Murine Anti-DLL3 Antibodies

Anti-DLL3 murine antibodies were produced as follows. In a first immunization campaign, three mice (one from each of the following strains: Balb/c, CD-1, FVB) were inoculated with human DLL3-fc protein (hDLL3-Fc) emulsified with an equal volume of TiterMax® or alum adjuvant. The hDLL3-Fc fusion construct was purchased from Adipogen International (Catalog No. AG-40A-0113). An initial immunization was performed with an emulsion of 10 μg hDLL3-Fc per mouse in TiterMax. Mice were then boosted biweekly with 5 μg hDLL3-Fc per mouse in alum adjuvant. The final injection prior to fusion was with 5 μg hDLL3-Fc per mouse in PBS.

In a second immunization campaign six mice (two each of the following strains: Balb/c, CD-1, FVB), were inoculated with human DLL3-His protein (hDLL3-His), emulsified with an equal volume of TiterMax® or alum adjuvant. Recombinant hDLL3-His protein was purified from the supernatants of CHO-S cells engineered to overexpress hDLL3-His. The initial immunization was with an emulsion of 10 μg hDLL3-His per mouse in TiterMax. Mice were then boosted biweekly with 5 μg hDLL3-His per mouse in alum adjuvant. The final injection was with 2×10⁵ HEK-293T cells engineered to overexpress hDLL3.

Solid-phase ELISA assays were used to screen mouse sera for mouse IgG antibodies specific for human DLL3. A positive signal above background was indicative of antibodies specific for DLL3. Briefly, 96 well plates (VWR International, Cat. #610744) were coated with recombinant DLL3-His at 0.5 μg/ml in ELISA coating buffer overnight. After washing with PBS containing 0.02% (v/v) Tween 20, the wells were blocked with 3% (w/v) BSA in PBS, 200 μL/well for 1 hour at room temperature (RT). Mouse serum was titrated (1:100, 1:200, 1:400, and 1:800) and added to the DLL3 coated plates at 50 μL/well and incubated at RT for 1 hour. The plates are washed and then incubated with 50 μL/well HRP-labeled goat anti-mouse IgG diluted 1:10,000 in 3% BSA-PBS or 2% FCS in PBS for 1 hour at RT. Again the plates were washed and 40 μL/well of a TMB substrate solution (Thermo Scientific 34028) was added for 15 minutes at RT. After developing, an equal volume of 2N H₂SO₄ was added to stop substrate development and the plates were analyzed by spectrophotometer at OD 450.

Sera-positive immunized mice were sacrificed and draining lymph nodes (popliteal, inguinal, and medial iliac) were dissected and used as a source for antibody producing cells. Cell suspensions of B cells (approximately 229×10⁶ cells from the hDLL3-Fc immunized mice, and 510×10⁶ cells from the hDLL3-His immunized mice) were fused with non-secreting P3×63Ag8.653 myeloma cells at a ratio of 1:1 by electro cell fusion using a model BTX Hybrimmune System (BTX Harvard Apparatus). Cells were re-suspended in hybridoma selection medium consisting of DMEM medium supplemented with azaserine, 15% fetal clone I serum, 10% BM Condimed (Roche Applied Sciences), 1 mM nonessential amino acids, 1 mM HEPES, 100 IU penicillin-streptomycin, and 50 μM 2-mercaptoethanol, and were cultured in four T225 flasks in 100 mL selection medium per flask. The flasks were placed in a humidified 370 incubator containing 5% CO₂ and 95% air for six to seven days.

On day six or seven after the fusions the hybridoma library cells were collected from the flasks and plated at one cell per well (using the FACSAria I cell sorter) in 200 μL of supplemented hybridoma selection medium (as described above) into 64 Falcon 96-well plates, and 48 96-well plates for the hDLL3-His immunization campaign. The rest of the library was stored in liquid nitrogen.

The hybridomas were cultured for 10 days and the supernatants were screened for antibodies specific to hDLL3 using flow cytometry performed as follows. 1×10⁵ per well of HEK-293T cells engineered to overexpress human DLL3, mouse DLL3 (pre-stained with dye), or cynomolgus DLL3 (pre-stained with Dylight 800) were incubated for 30 minutes with 25 μL hybridoma supernatant. Cells were washed with PBS/2% FCS and then incubated with 25 μL per sample DyeLight 649 labeled goat-anti-mouse IgG, Fc fragment specific secondary diluted 1:300 in PBS/2% FCS. After a 15 minute incubation cells were washed twice with PBS/2% FCS and re-suspended in PBS/2% FCS with DAPI and analyzed by flow cytometry for fluorescence exceeding that of cells stained with an isotype control antibody. Remaining unused hybridoma library cells were frozen in liquid nitrogen for future library testing and screening.

The hDLL3-His immunization campaign yielded approximately 50 murine anti-hDLL3 antibodies and the hDLL3-Fc immunization campaign yielded approximately 90 murine anti-hDLL3 antibodies.

### Example 2

### Sequencing of Anti-DLL3 Antibodies

Based on the foregoing, a number of exemplary distinct monoclonal antibodies that bind immobilized human DLL3 or h293-hDLL3 cells with apparently high affinity were selected for sequencing and further analysis. Sequence analysis of the light chain variable regions and heavy chain variable regions from selected monoclonal antibodies generated in Example 1 confirmed that many had novel complementarity determining regions and often displayed novel VDJ arrangements.

Initially selected hybridoma cells expressing the desired antibodies were lysed in Trizol® reagent (Trizol® Plus RNA Purification System, Life Technologies) to prepare the RNA encoding the antibodies. Between 10⁴ and 10⁵ cells were re-suspended in 1 mL Trizol and shaken vigorously after addition of 200 μL chloroform. Samples were then centrifuged at 4° C. for 10 minutes and the aqueous phase was transferred to a fresh microfuge tube and an equal volume of 70% ethanol was added. The sample was loaded on an RNeasy Mini spin column, placed in a 2 mL collection tube and processed according to the manufacturer's instructions. Total RNA was extracted by elution, directly to the spin column membrane with 100 μL RNase-free water. The quality of the RNA preparations was determined by fractionating 3 μL in a 1% agarose gel before being stored at −80° C. until used.

The variable region of the Ig heavy chain of each hybridoma was amplified using a 5′ primer mix comprising 32 mouse specific leader sequence primers designed to target the complete mouse VH repertoire in combination with a 3′ mouse Cγ primer specific for all mouse Ig isotypes. Similarly, a primer mix containing thirty two 5′ Vκ leader sequences designed to amplify each of the Vκ mouse families was used in combination with a single reverse primer specific to the mouse kappa constant region in order to amplify and sequence the kappa light chain. For antibodies containing a lambda light chain, amplification was performed using three 5′ VL leader sequences in combination with one reverse primer specific to the mouse lambda constant region. The VH and VL transcripts were amplified from 100 ng total RNA using the Qiagen One Step RT-PCR kit as follows. A total of eight RT-PCR reactions were run for each hybridoma, four for the Vκ light chain and four for the Vγ heavy chain. PCR reaction mixtures included 3 μL of RNA, 0.5 μL of 100 μM of either heavy chain or kappa light chain primers (custom synthesized by Integrated Data Technologies), 5 μL of 5×RT-PCR buffer, 1 μL dNTPs, 1 μL of enzyme mix containing reverse transcriptase and DNA polymerase, and 0.4 μL of ribonuclease inhibitor RNasin (1 unit). The thermal cycler program was RT step 500 for 30 minutes, 950 for 15 minutes followed by 30 cycles of (950 for 30 seconds, 48° C. for seconds, 720 for 1 minute). There was then a final incubation at 720 for 10 minutes.

The extracted PCR products were sequenced using the same specific variable region primers as described above for the amplification of the variable regions. To prepare the PCR products for direct DNA sequencing, they were purified using the QIAquick™ PCR Purification Kit (Qiagen) according to the manufacturer's protocol. The DNA was eluted from the spin column using 50 μL of sterile water and then sequenced directly from both strands (MCLAB).

Selected nucleotide sequences were analyzed using the IMGT sequence analysis tool (http//www.imgt.org/IMGTmedicaV sequence_analysis.html) to identify germline V, D and J gene members with the highest sequence homology. These derived sequences were compared to known germline DNA sequences of the Ig V- and J-regions by alignment of VH and VL genes to the mouse germline database using a proprietary antibody sequence database.

FIG. 1A depicts the contiguous amino acid sequences of numerous novel murine light chain variable regions from anti-DLL3 antibodies and exemplary humanized light chain variable regions derived from the variable light chains of representative murine anti-DLL3 antibodies (as per Example 3 below). FIG. 1B depicts the contiguous amino acid sequences of novel murine heavy chain variable regions from the same anti-DLL3 antibodies and humanized heavy chain variable regions derived from the same murine antibodies providing the humanized light chains (as per Example 3 below). Murine light and heavy chain variable region amino acid sequences are provided in SEQ ID NOS: 21-387, odd numbers while humanized light and heavy chain variable region amino acid sequences are provided in SEQ ID NOS: 389-407, odd numbers.

Thus, taken together FIGS. 1A and 1B provide the annotated sequences of numerous murine anti-DLL3 binding or targeting domains, termed SC16.3, SC16.4, SC16.5, SC16.7, SC16.8, SC16.10, SC16.11, SC16.13, SC16.15, SC16.18, SC16.19, SC16.20, SC16.21, SC16.22, SC16.23, SC16.25, SC16.26, SC16.29, SC16.30, SC16.31, SC16.34, SC16.35, SC16.36, SC16.38, SC16.41, SC16.42, SC16.45, SC16.47, SC16.49, SC16.50, SC16.52, SC16.55, SC16.56, SC16.57, SC16.58, SC16.61, SC16.62, SC16.63, SC16.65, SC16.67, SC16.68, SC16.72, SC16.73, SC16.78, SC16.79, SC16.80, SC16.81, SC16.84, SC16.88, SC16.101, SC16.103, SC16.104, SC16.105, SC16.106, SC16.107, SC16.108, SC16.109, SC16.110, SC16.111, SC16.113, SC16.114, SC16.115, SC16.116, SC16.117, SC16.118, SC16.120, SC16.121, SC16.122, SC16.123, SC16.124, SC16.125, SC16.126, SC16.129, SC16.130, SC16.131, SC16.132, SC16.133, SC16.134, SC16.135, SC16.136, SC16.137, SC16.138, SC16.139, SC16.140, SC16.141, SC16.142, SC16.143, SC16.144, SC16.147, SC16.148, SC16.149 and SC16.150 and humanized antibodies, termed hSC16.13, hSC16.15, hSC16.25, hSC16.34 and hSC16.56.

In particular aspects of the invention the CAR binding domain binds specifically to hDLL3 and was derived from, comprises or competes for binding with an antibody comprising: a light chain variable region (VL) of SEQ ID NO: 21 and a heavy chain variable region (VH) of SEQ ID NO: 23; or a VL of SEQ ID NO: 25 and a VH of SEQ ID NO: 27; or a VL of SEQ ID NO: 29 and a VH of SEQ ID NO: 31; or a VL of SEQ ID NO: 33 and a VH of SEQ ID NO: 35; or a VL of SEQ ID NO: 37 and a VH of SEQ ID NO: 39; or a VL of SEQ ID NO: 41 and a VH of SEQ ID NO: 43; or a VL of SEQ ID NO: 45 and a VH of SEQ ID NO: 47; or a VL of SEQ ID NO: 49 and a VH of SEQ ID NO: 51; or a VL of SEQ ID NO: 53 and a VH of SEQ ID NO: 55; or a VL of SEQ ID NO: 57 and a VH of SEQ ID NO: 59; or a VL of SEQ ID NO: 61 and a VH of SEQ ID NO: 63; or a VL of SEQ ID NO: 65 and a VH of SEQ ID NO: 67; or a VL of SEQ ID NO: 69 and a VH of SEQ ID NO: 71; or a VL of SEQ ID NO: 73 and a VH of SEQ ID NO: 75; or a VL of SEQ ID NO: 77 and a VH of SEQ ID NO: 79; or a VL of SEQ ID NO: 81 and a VH of SEQ ID NO: 83; or a VL of SEQ ID NO: 85 and a VH of SEQ ID NO: 87; or a VL of SEQ ID NO: 89 and a VH of SEQ ID NO: 91; or a VL of SEQ ID NO: 93 and a VH of SEQ ID NO: 95; or a VL of SEQ ID NO: 97 and a VH of SEQ ID NO: 99; or a VL of SEQ ID NO: 101 and a VH of SEQ ID NO: 103; or a VL of SEQ ID NO: 105 and a VH of SEQ ID NO: 107; or a VL of SEQ ID NO: 109 and a VH of SEQ ID NO: 111; or a VL of SEQ ID NO: 113 and a VH of SEQ ID NO: 115; or a VL of SEQ ID NO: 117 and a VH of SEQ ID NO: 119; or a VL of SEQ ID NO: 121 and a VH of SEQ ID NO: 123; or a VL of SEQ ID NO: 125 and a VH of SEQ ID NO: 127; or a VL of SEQ ID NO: 129 and a VH of SEQ ID NO: 131; or a VL of SEQ ID NO: 133 and a VH of SEQ ID NO: 135; or a VL of SEQ ID NO: 137 and a VH of SEQ ID NO: 139; or a VL of SEQ ID NO: 141 and a VH of SEQ ID NO: 143; or a VL of SEQ ID NO: 145 and a VH of SEQ ID NO: 147; or a VL of SEQ ID NO: 149 and a VH of SEQ ID NO: 151; or a VL of SEQ ID NO: 153 and a VH of SEQ ID NO: 155; or a VL of SEQ ID NO: 157 and a VH of SEQ ID NO: 159; or a VL of SEQ ID NO: 161 and a VH of SEQ ID NO: 163; or a VL of SEQ ID NO: 165 and a VH of SEQ ID NO: 167; or a VL of SEQ ID NO: 169 and a VH of SEQ ID NO: 171; or a VL of SEQ ID NO: 173 and a VH of SEQ ID NO: 175; or a VL of SEQ ID NO: 177 and a VH of SEQ ID NO: 179; or a VL of SEQ ID NO: 181 and a VH of SEQ ID NO: 183; or a VL of SEQ ID NO: 185 and a VH of SEQ ID NO: 187; or a VL of SEQ ID NO: 189 and a VH of SEQ ID NO: 191; or a VL of SEQ ID NO: 193 and a VH of SEQ ID NO: 195; or a VL of SEQ ID NO: 197 and a VH of SEQ ID NO: 199; or a VL of SEQ ID NO: 201 and a VH of SEQ ID NO: 203; or a VL of SEQ ID NO: 205 and a VH of SEQ ID NO: 207; or a VL of SEQ ID NO: 209 and a VH of SEQ ID NO: 211; or a VL of SEQ ID NO: 213 and a VH of SEQ ID NO: 215; or a VL of SEQ ID NO: 217 and a VH of SEQ ID NO: 219; or a VL of SEQ ID NO: 221 and a VH of SEQ ID NO: 223; or a VL of SEQ ID NO: 225 and a VH of SEQ ID NO: 227; or a VL of SEQ ID NO: 229 and a VH of SEQ ID NO: 231; or a VL of SEQ ID NO: 233 and a VH of SEQ ID NO: 235; or a VL of SEQ ID NO: 237 and a VH of SEQ ID NO: 239; or a VL of SEQ ID NO: 241 and a VH of SEQ ID NO: 243; or a VL of SEQ ID NO: 245 and a VH of SEQ ID NO: 247; or a VL of SEQ ID NO: 249 and a VH of SEQ ID NO: 251; or a VL of SEQ ID NO: 253 and a VH of SEQ ID NO: 255; or a VL of SEQ ID NO: 257 and a VH of SEQ ID NO: 259; or a VL of SEQ ID NO: 261 and a VH of SEQ ID NO: 263; or a VL of SEQ ID NO: 265 and a VH of SEQ ID NO: 267; or a VL of SEQ ID NO: 269 and a VH of SEQ ID NO: 271; or a VL of SEQ ID NO: 273 and a VH of SEQ ID NO: 275; or a VL of SEQ ID NO: 277 and a VH of SEQ ID NO: 279; or a VL of SEQ ID NO: 281 and a VH of SEQ ID NO: 283; or a VL of SEQ ID NO: 285 and a VH of SEQ ID NO: 287; or a VL of SEQ ID NO: 289 and a VH of SEQ ID NO: 291; or a VL of SEQ ID NO: 293 and a VH of SEQ ID NO: 295; or a VL of SEQ ID NO: 297 and a VH of SEQ ID NO: 299; or a VL of SEQ ID NO: 301 and a VH of SEQ ID NO: 303; or a VL of SEQ ID NO: 305 and a VH of SEQ ID NO: 307; or a VL of SEQ ID NO: 309 and a VH of SEQ ID NO: 311; or a VL of SEQ ID NO: 313 and a VH of SEQ ID NO: 315; or a VL of SEQ ID NO: 317 and a VH of SEQ ID NO: 319; or a VL of SEQ ID NO: 321 and a VH of SEQ ID NO: 323; or a VL of SEQ ID NO: 325 and a VH of SEQ ID NO: 327; or a VL of SEQ ID NO: 329 and a VH of SEQ ID NO: 331; or a VL of SEQ ID NO: 333 and a VH of SEQ ID NO: 335; or a VL of SEQ ID NO: 337 and a VH of SEQ ID NO: 339; or a VL of SEQ ID NO: 341 and a VH of SEQ ID NO: 343; or a VL of SEQ ID NO: 345 and a VH of SEQ ID NO: 347; or a VL of SEQ ID NO: 349 and a VH of SEQ ID NO: 351; or a VL of SEQ ID NO: 353 and a VH of SEQ ID NO: 355; or a VL of SEQ ID NO: 357 and a VH of SEQ ID NO: 359; or a VL of SEQ ID NO: 361 and a VH of SEQ ID NO: 363; or a VL of SEQ ID NO: 365 and a VH of SEQ ID NO: 367; or a VL of SEQ ID NO: 369 and a VH of SEQ ID NO: 371; or a VL of SEQ ID NO: 373 and a VH of SEQ ID NO: 375; or a VL of SEQ ID NO: 377 and a VH of SEQ ID NO: 379; or a VL of SEQ ID NO: 381 and a VH of SEQ ID NO: 383; or a VL of SEQ ID NO: 385 and a VH of SEQ ID NO: 387; or a VL of SEQ ID NO: 389 and a VH of SEQ ID NO: 391; or a VL of SEQ ID NO: 393 and a VH of SEQ ID NO: 395; or a VL of SEQ ID NO: 397 and a VH of SEQ ID NO: 399; or a VL of SEQ ID NO: 401 and a VH of SEQ ID NO: 403; or a VL of SEQ ID NO: 405 and a VH of SEQ ID NO: 407.

For the purposes of the instant application the SEQ ID NOS of each particular antibody are sequential odd numbers. Thus the monoclonal anti-DLL3 antibody, SC16.3, comprises amino acid SEQ ID NOS: 21 and 23 for the light and heavy chain variable regions respectively; SC16.4 comprises SEQ ID NOS: 25 and 27; SC16.5 comprises SEQ ID NOS: 29 and 31, and so on. A corresponding nucleic acid sequence encoding each antibody amino acid sequence is included in the appended sequence listing and has the SEQ ID NO immediately preceding the corresponding amino acid SEQ ID NO: Thus, for example, the SEQ ID NOS of the VL and VH of the SC16.3 antibody are 21 and 23 respectively, and the SEQ ID NOS of the nucleic acid sequences encoding the VL and VH of the SC16.3 antibody are SEQ ID NOS: 20 and 22 respectively.

It should be noted that, due to sequencing anomalies, certain heavy and light chain variable region sequences were prematurely truncated during the sequencing process. This resulted in the omission of one or more amino acids in the reported FR4 sequence. In such cases compatible amino acids (determined by review of corresponding sequences from other antibody clones) have been supplied to essentially complete the variable region sequence. For example, the residues “IK” were added to the terminal end of the SC16.22 light chain sequence in FIG. 1A (SEQ ID NO: 73) to provide an operable light chain variable region with a complete framework 4. Bases encoding the added amino acids were similarly added to the corresponding nucleic acid sequence (SEQ ID NO: 72) to ensure consistency. In each such case in FIGS. 1A and 1B (but not in the appended sequence listing) the added amino acids are underlined and bolded so as to be readily identified. The CDRs in FIGS. 1A and 1B are defined as per Kabat et al. (supra) using a proprietary version of the Abysis database.

### Example 3

### Generation of Chimeric and Humanized Anti-DLL3 Antibodies

To provide a benchmark for humanized binding domains compatible with the instant invention five (e.g. SC16.13, SC16.15, SC16.25, SC16.34 and SC16.56) exemplary chimeric anti-DLL3 antibodies were generated using art-recognized techniques as follows. Total RNA was extracted from the hybridomas and amplified as set forth in Example 1. Data regarding V, D and J gene segments of the VH and VL chains of the murine antibodies were obtained from the derived nucleic acid sequences. Primer sets specific to the leader sequence of the VH and VL chain of the antibody were designed using the following restriction sites: AgeI and XhoI for the VH fragments, and XmaI and DraIII for the VL fragments. PCR products were purified with a QIAquick PCR purification kit (Qiagen), followed by digestion with restriction enzymes AgeI and XhoI for the VH fragments and XmaI and DraIII for the VL fragments. The VL and VH digested PCR products were purified and ligated into kappa CL (SEQ ID NO: 5) human light chain constant region expression vector or IgG1 (SEQ ID NO: 6) human heavy chain constant region expression vector, respectively.

Ligation reactions were performed in a total volume of 10 μL with 200U T4-DNA Ligase (New England Biolabs), 7.5 μL of digested and purified gene-specific PCR product and 25 ng linearized vector DNA. Competent E. coli DH10B bacteria (Life Technologies) were transformed via heat shock at 42° C. with 3 μL ligation product and plated onto plates with ampicillin at a concentration of 100 μg/mL. Following purification and digestion of the amplified ligation products, the VH fragment was cloned into the AgeI-XhoI restriction sites of the pEE6.4HuIgG1 expression vector (Lonza) and the VL fragment was cloned into the XmaI-DraIII restriction sites of the pEE12.4Hu-Kappa expression vector (Lonza).

Chimeric antibodies were expressed by co-transfection of HEK-293T cells with pEE6.4HuIgG1 and pEE12.4Hu-Kappa expression vectors. Prior to transfection the HEK-293T cells were cultured in 150 mm plates under standard conditions in Dulbecco's Modified Eagle's Medium (DMEM) supplemented with 10% heat inactivated FCS, 100 μg/mL streptomycin and 100 U/mL penicillin G. For transient transfections cells were grown to 80% confluency. 12.5 μg each of pEE6.4HuIgG1 and pEE12.4Hu-Kappa vector DNA were added to 50 μL HEK-293T transfection reagent in 1.5 mL Opti-MEM. The mix was incubated for 30 minutes at room temperature and plated. Supernatants were harvested three to six days after transfection. Culture supernatants containing recombinant chimeric antibodies were cleared from cell debris by centrifugation at 800×g for 10 minutes and stored at 4° C. Recombinant chimeric antibodies were purified by Protein A affinity chromatography.

The same murine anti-DLL3 antibodies (e.g. SC16.13, SC16.15, SC16.25, SC16.34 and SC16.56) were also used to derive CDR-grafted or humanized binding domains. The murine antibodies were humanized using a proprietary computer-aided CDR-grafting method (Abysis Database, UCL Business) and standard molecular engineering techniques as follows. Human framework regions of the variable regions were designed based on the highest homology between the framework sequences and CDR canonical structures of human germline antibody sequences and the framework sequences and CDRs of the relevant mouse antibodies. For the purpose of the analysis the assignment of amino acids to each of the CDR domains was done in accordance with Kabat et al. Once the variable regions were selected, they were generated from synthetic gene segments (Integrated DNA Technologies). Humanized antibodies were cloned and expressed using the molecular methods described above for chimeric antibodies.

The genetic composition for the selected human acceptor variable regions are shown in TABLE 4 immediately below for each of the humanized antibodies. The sequences depicted in TABLE 4 correspond to the contiguous variable region amino acid sequences set forth in SEQ ID NOS: 389 and 391 (hSC16.13), SEQ ID NOS: 393 and 395 (hSC16.15), SEQ ID NOS: 397 and 399 (hSC16.25), SEQ ID NOS: 401 and 403 (hSC16.34) and SEQ ID NOS: 405 and 407 (hSC16.56) in FIGS. 1A and 1B. TABLE 4 shows that no framework changes or back mutations were necessary to maintain the favorable binding properties of the selected antibodies.

TABLE 4

| mAb | human VH | human DH | human JH | FW changes | human VK | human JK | FW changes |
|---|---|---|---|---|---|---|---|
| hSC16.13 | IGHV2-5*01 | IGHD1-1 | JH6 | None | IGKV1-39*01 | JK1 | None |
| hSC16.15 | IGHV1-46*01 | IGHD2-2 | JH4 | None | IGKV1-13*02 | JK4 | None |
| hSC16.25 | IGHV2-5*01 | IGHD3-16 | JH6 | None | IGKV6-21*01 | JK2 | None |
| hSC16.34 | IGHV1-3*02 | IGHD3-22 | JH4 | None | IGKV1-27*01 | JK1 | None |
| hSC16.56 | IGHV1-18*01 | IGHD2-21 | JH4 | None | IGKV3-15*01 | JK2 | None |

Although no residues were altered in the framework regions, in one of the humanized clones (hSC16.13) mutations were introduced into heavy chain CDR2 to address stability concerns. The binding affinity of the antibody with the modified CDR was checked to ensure that it was equivalent to either the corresponding chimeric or murine antibody.

Following humanization of the selected antibodies the resulting VL and VH chain amino acid sequences were analyzed to determine their homology with regard to the murine donor and human acceptor light and heavy chain variable regions. The results shown in TABLE 5, immediately below, reveal that the humanized constructs consistently exhibited a higher homology with respect to the human acceptor sequences than with the murine donor sequences. The murine heavy and light chain variable regions show a similar overall percentage homology to a closest match of human germline genes (85%-93%) compared with the homology of the humanized antibodies and the donor hybridoma protein sequences (74%-83%).

TABLE 5

| | Homology to Human | Homology to Murine Parent |
|---|---|---|
| mAb | (CDR acceptor) | (CDR donor) |
| hSC16.13 HC | 93% | 81% |
| hSC16.13 LC | 87% | 77% |
| hSC16.15 HC | 85% | 83% |
| hSC16.15 LC | 85% | 83% |
| hSC16.25 HC | 91% | 83% |
| hSC16.25 LC | 85% | 79% |
| hSC16.34 HC | 87% | 79% |
| hSC16.34 LC | 85% | 81% |
| hSC16.56 HC | 87% | 74% |
| hSC16.56 LC | 87% | 76% |

Each of the derived humanized constructs were analyzed using surface plasmon resonance, to determine if the CDR grafting process had appreciably altered their apparent affinity for DLL3 protein. The humanized constructs were compared with chimeric antibodies comprising the murine parent (or donor) heavy and light chain variable domains and a human constant region substantially equivalent to that used in the humanized constructs. The humanized anti-DLL3 antibodies exhibited binding characteristics roughly comparable to those shown by the chimeric parent antibodies (data not shown).

### Example 4

### Domain and Epitope Mapping of Anti-DLL3 Antibodies

In order to characterize and position the epitopes that the disclosed anti-DLL3 antibodies bind to, domain-level epitope mapping was performed using a modification of the protocol described by Cochran et al., 2004 (supra). Individual domains of DLL3 comprising specific amino acid sequences were expressed on the surface of yeast, and binding by each anti-DLL3 antibody was determined through flow cytometry.

Yeast display plasmid constructs were created for the expression of the following constructs: DLL3 extracellular domain (amino acids 27-466); DLL1-DLL3 chimera, which consists of the N-terminal region and DSL domain of DLL1 (amino acids 22-225) fused to EGF-like domains 1 through 6 of DLL3 (amino acids 220-466); DLL3-DLL1 chimera, which consists of the N-terminal region and DSL domain of DLL3 (amino acids 27-214) fused to EGF-like domains 1 through 8 of DLL1 (amino acids 222-518); EGF1 (amino acids 215-249); EGF2 (amino acids 274-310); EGF1 and EGF2 (amino acids 215-310); EGF3 (amino acids 312-351); EGF4 (amino acids 353-389); EGF5 (amino acids 391-427); and EGF6 (amino acids 429-465). For domain information see generally UniProtKB/Swiss-Prot database entry Q9NYJ7. Note that the amino acid numbering references an unprocessed DLL3 protein with a leader sequence included in the sequence set forth in SEQ ID NO: 1). For analysis of the N-terminal region or the EGF domains as a whole, chimeras with the family member DLL1 (DLL1-DLL3 and DLL3-DLL1) were used as opposed to fragments to minimize potential problems with protein folding. Domain-mapped antibodies had previously been shown not to cross-react with DLL1 indicating that any binding to these constructs was occurring through association with the DLL3 portion of the construct. These plasmids were transformed into yeast, which were then grown and induced as described in Cochran et al.

To test for binding to a particular construct, 200,000 induced yeast cells expressing the desired construct were washed twice in PBS+1 mg/mL BSA (PBSA), and incubated in 50 μL of PBSA with biotinylated anti-HA clone 3F10 (Roche Diagnostics) at 0.1 μg/mL and either 50 nM purified antibody or 1:2 dilution of unpurified supernatant from hybridomas cultured for 7 days. Cells were incubated for 90 minutes on ice, followed by two washes in PBSA. Cells were then incubated in 50 μL PBSA with the appropriate secondary antibodies: for murine antibodies, Alexa 488 conjugated streptavidin, and Alexa 647 conjugated goat anti mouse (Life Technologies) were added at 1 μg/mL each; and for humanized or chimeric antibodies, Alexa 647 conjugated streptavidin (Life Technologies) and R-phycoerythrin conjugated goat anti human (Jackson Immunoresearch) were added at 1 μg/mL each. After a twenty minute incubation on ice, cells were washed twice with PBSA and analyzed on a FACS Canto II. Antibodies that bound to DLL3-DLL1 chimera were designated as binding to the N-terminal region+DSL. Antibodies that bound specifically to an epitope present on a particular EGF-like domain were designated as binding to its respective domain (FIG. 2.)

In order to classify an epitope as conformational (e.g., discontinuous) or linear, yeast displaying the DLL3 ECD was heat treated for 30 minutes at 80° C. to denature the DLL3 ECD, and then washed twice in ice-cold PBSA. The ability of anti-DLL3 antibodies to bind the denatured yeast was tested by FACS using the same staining protocol as described above. Antibodies that bound to both the denatured and native yeast were classified as binding to a linear epitope, whereas antibodies that bound native yeast but not denatured yeast were classified as conformationally specific.

A schematic summary of the domain-level epitope mapping data of the antibodies tested is presented in FIG. 2, with antibodies binding a linear epitope underlined and, where determined, the corresponding bin noted in parenthesis. A review of FIG. 2 shows that the majority of anti-DLL3 antibodies tended to map to epitopes found either in the N-terminal/DSL region of DLL3 or EGF2.

Fine epitope mapping was further performed on selected antibodies using one of two methods. The first method employed the Ph.D.-12 phage display peptide library kit (New England Biolabs) which was used in accordance with the manufacturer's instructions. The antibody for epitope mapping was coated overnight at 50 μg/mL in 3 mL 0.1 M sodium bicarbonate solution, pH 8, onto a Nunc MaxiSorp tube (Nunc). The tube was blocked with 3% BSA solution in bicarbonate solution. Then, 10¹¹ input phage in PBS+0.1% Tween-20 was allowed to bind, followed by ten consecutive washes with 0.1% Tween-20 to wash away non-binding phage. Remaining phage were eluted with 1 mL 0.2 M glycine for 10 minutes at room temperature with gentle agitation, followed by neutralization with 150 μL 1M Tris-HCl pH 9. Eluted phage were amplified and panned again with 10¹¹ input phage, using 0.5% Tween-20 during the wash steps to increase selection stringency. DNA from 24 plaques of the eluted phage from the second round was isolated using the Qiaprep M13 Spin kit (Qiagen) and sequenced. Binding of clonal phage was confirmed using an ELISA assay, where the mapped antibody or a control antibody was coated onto an ELISA plate, blocked, and exposed to each phage clone. Phage binding was detected using horseradish peroxidase conjugated anti-M13 antibody (GE Healthcare), and the 1-Step Turbo TMB ELISA solution (Pierce). Phage peptide sequences from specifically binding phage were aligned using Vector NTI (Life Technologies) against the antigen ECD peptide sequence to determine the epitope of binding.

Alternatively, a yeast display method (Chao et al., 2007, PMID: 17406305) was used to map the epitopes of selected antibodies. Libraries of DLL3 ECD mutants were generated with error prone PCR using nucleotide analogues 8-oxo-2′deoxyguanosine-5′-triphosphate and 2′-deoxy-p-nucleoside-5′triphosphate (TriLink Bio) for a target mutagenesis rate of one amino acid mutation per clone. These were transformed into a yeast display format. Using the technique described above for domain-level mapping, the library was stained for HA and antibody binding at 50 nM. Using a FACS Aria (BD), clones that exhibited a loss of binding compared to wild type DLL3 ECD were sorted. These clones were re-grown, and subjected to another round of FACS sorting for loss of binding to the target antibody. Using the Zymoprep Yeast Plasmid Miniprep kit (Zymo Research), individual ECD clones were isolated and sequenced. Where necessary, mutations were reformatted as single-mutant ECD clones using the Quikchange site directed mutagenesis kit (Agilent).

Individual ECD clones were next screened to determine whether loss of binding was due to a mutation in the epitope, or a mutation that caused misfolding. Mutations that involved cysteine, proline, and stop codons were automatically discarded due to the high likelihood of a misfolding mutation. Remaining ECD clones were then screened for binding to a non-competing, conformationally specific antibody. ECD clones that lost binding to non-competing, conformationally specific antibodies were concluded to contain misfolding mutations, whereas ECD clones that retained equivalent binding to wild type DLL3 ECD were concluded to be properly folded. Mutations in the ECD clones in the latter group were concluded to be in the epitope.

A summary of selected antibodies with their derived epitopes comprising amino acid residues that are involved in antibody binding are listed in TABLE 6 below. Antibodies SC16.34 and SC16.56 interact with common amino acid residues which is consistent with the binning information and domain mapping results shown in FIG. 2. Moreover, SC16.23 was found to interact with a distinct contiguous epitope and was found not to bin with SC16.34 or SC16.56. Note that for the purposes of the appended sequence listing SEQ ID NO: 4 comprises a placeholder amino acid at position 204.

TABLE 6

| Antibody Clone | Epitope | SEQ ID NO: |
|---|---|---|
| SC16.23 | Q93, P94, G95, A96, P97 | 3 |
| SC16.34 | G203, R205, P206 | 4 |
| SC16.56 | G203, R205, P206 | 4 |

### Example 5

### Generation of an Anti-DLL3 Chimeric Antigen Receptor

Fabrication of an Anti-CD19 CAR

To generate a positive control CAR construct, a synthetic open reading frame encoding a second generation CAR directed towards human CD19 (see US2014/0271635) was synthesized (Life Technologies) and subcloned into the multiple cloning site (MCS) of the lentiviral expression vector pCDH-CMV-MCS-EF1-GFP-T2A-Puro (System Biosciences, Mountain View Calif.). This anti-CD19 CAR open reading frame comprises nucleotides, from 5′ to 3′, encoding the signal leader sequence from the human CD8 alpha chain (amino acids 1-21, UniProt accession P01732-1), a scFv derived from a mouse monoclonal antibody recognizing human CD19 (Nicholson et al, 1997; PMID 9566763), the human CD8 alpha hinge, transmembrane domain and proximal region (amino acids 138-206, UniProt accession P01732-1), the intracellular costimulatory signaling region from the human 4-1BB protein (amino acids 214-255, UniProt accession Q07011-1), and the human CD3_{ζ} chain intracellular signaling region (amino acids 52-164, UniProt accession P20963-1 with a Q65K modification). When expressed on lymphocytes the resulting CD19 CAR-T exhibited the expected immunostimulatory activity.

Besides providing a positive control the anti-CD19 CAR/lentiviral expression vector was designed with restriction sites in such a way that the anti-CD19 scFv component could be easily removed and substituted with an alternative binding region component directed to any selected determinant (e.g., DLL3). As described below, this cassette system (designated SCT1-XX where XX indicates the particular DLL3 binding domain component) was used to validate various embodiments of the instant invention. Note that the SCT nomenclature may, depending on the context, refer to the expressed anti-DLL3 CAR protein, cytotoxic lymphocytes expressing the CAR protein, the anti-DLL3 CAR ORF or an expression vector (e.g., lentiviral, retroviral, plasmid, etc.) comprising the same ORF.

Fabrication of SCT1-h16.15.

To generate a novel anti-DLL3 CAR construct (SCT1-h16.15), a nucleotide sequence encoding an scFv fragment was first synthesized by operably linking anti-hSC16.15 VL (SEQ ID NO: 394) and VH (SEQ ID NO: 396) nucleotide sequences together via a nucleic acid sequence encoding a pentameric multimer GlyGlyGlyGlySer (G₄S)₃ (GGGGSGGGGSGGGGS; SEQ ID NO: 7) linker to provide a hSC16.15-scFv polynucleotide (SEQ ID NO: 15) that encodes the hSC16.15-scFv protein. Both exemplary nucleic acid and amino acid sequences are set forth immediately below:

(SEQ ID NO: 8)
AIQLTQSPSSLSASVGDRVTITCRASENIYYNLAWYQQKPGKAPKLLIYT
ANSLEDVPSRFSGSGSGTDFTLTISSLQPEDFATYFCKQAYDVPPTFGGG
TKLEIKGGGGSGGGGSGGGGSQVQLVQSGAEVKKPGASVKVSCKASGYTF
TRYWIHWIRQAPGQGLEWMGYINPTVYTEFNQNFKDRVTMTRDTSTSTVY
MELSSLRSEDTAVYYCARGGSNFFDYWQGTTVTVSS
and:
(SEQ ID NO: 15)
gccatccagttgacccagtctccatcctccctgtctgcatctgtaggaga
cagagtcaccatcacttgccgggcaagtgagaacatttactacaatttag
cctggtatcagcagaaaccagggaaagctcctaagctcctgatctatact
gccaatagtttggaagatggggtcccatcaaggttcagcggcagtggatc
tgggacagatttcactctcaccatcagcagcctgcagcctgaagattttg
caacttatttttgtaaacaggcttatgacgttcctccgacgttcggtgga
ggcaccaagctggaaatcaaaggcggcggaggatctggcggaggcggaag
tggcggagggggatctcaggtgcagctggtgcagtctggggctgaggtga
agaagcctggggcctcagtgaaggtttcctgcaaggcatctggatacacc
ttcaccaggtactggatacactggatacgacaggcccctggacaagggct
tgagtggatgggatacatcaaccctacaactgtttatactgagttcaatc
agaacttcaaggacagagtcaccatgaccagggacacgtccacgagcaca
gtctacatggagctgagcagcctgagatctgaggacacggccgtgtatta
ctgtgcgagaggcggtagtaacttctttgactactggggccaaggcacca
ctgtcacagtctcctcg.

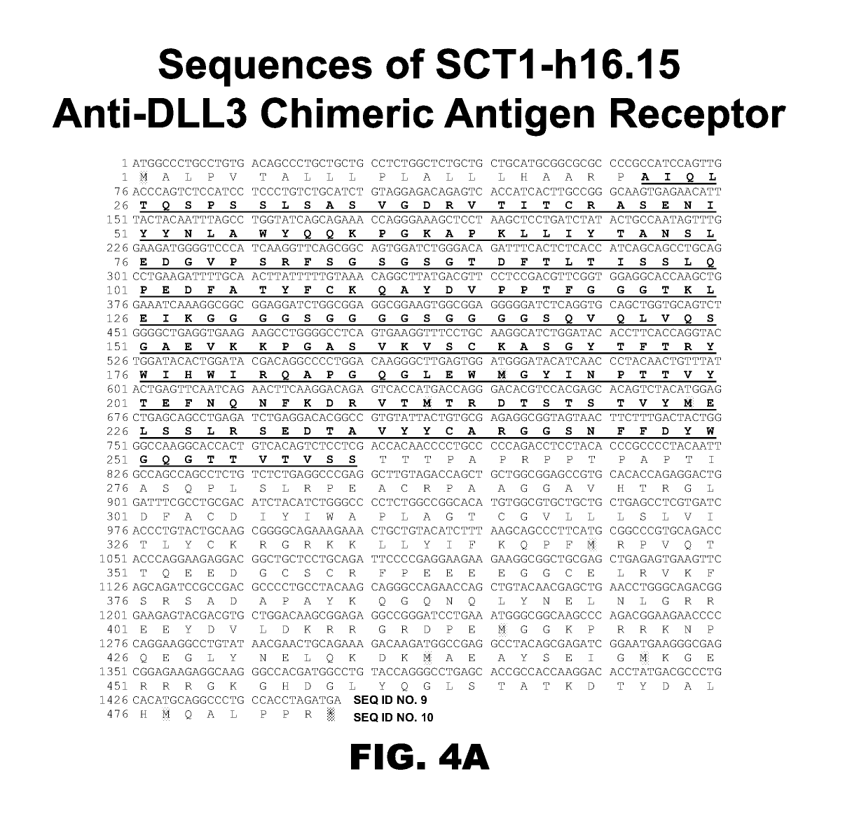

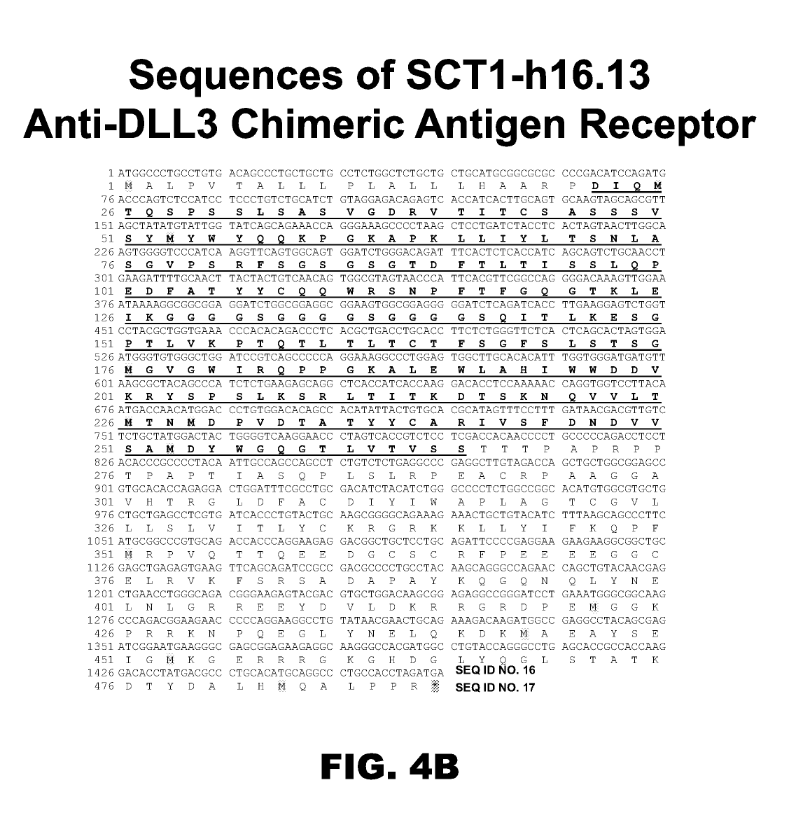

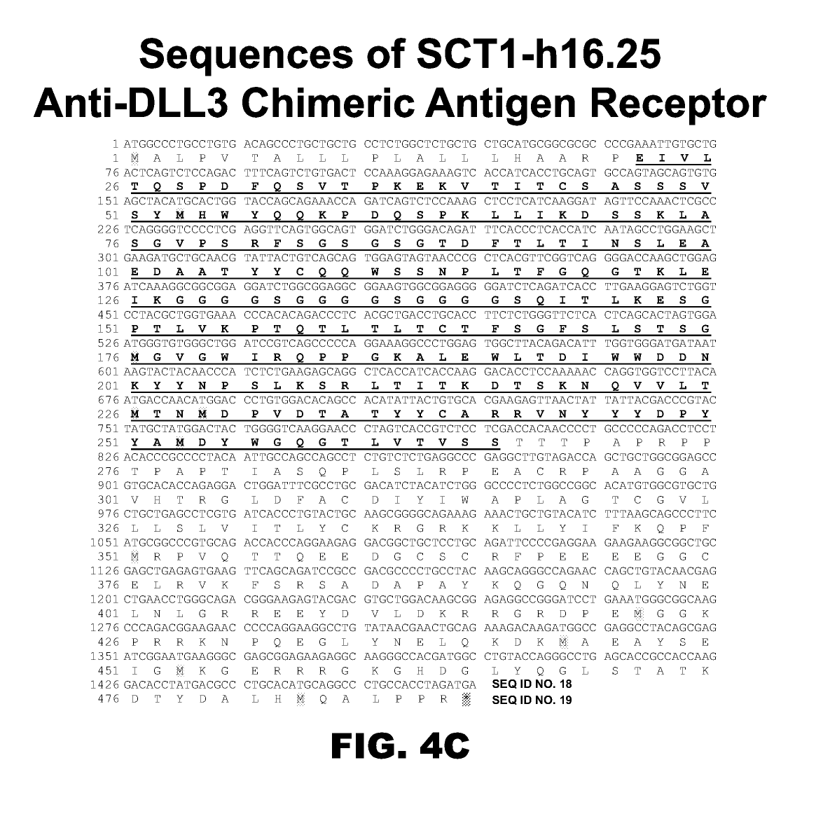

Using standard molecular engineering techniques the hSC16.15-scFv nucleotide sequence was subsequently cloned into the SCT1 cassette to provide a SCT1-h16.15 lentiviral expression vector comprising an anti-DLL3 CAR. In this regard the SCT1-h16.15 CAR comprises an open reading frame encoding the following elements from 5′ to 3′: CD8 alpha chain leader region (amino acids 1-21, UniProt P01732-1), h16.15 VL domain (as per Example 3), (G₄S)₃ synthetic linker sequence (amino acid 1-15, Huston et al., 1988), h16.15 VH domain (as per Example 3), the human CD8 alpha hinge and transmembrane domain (amino acids 138-206, UniProt accession P01732-1), the intracellular costimulatory signaling region from the human 4-1BB protein (amino acids 214-255, UniProt accession Q07011-1) and the human CD3c chain intracellular signaling region (amino acids 52-164, UniProt accession P20963-1 with a Q65K modification). The CAR open reading frame was sequence confirmed. A schematic diagram of the SCT1-h16.15 CAR open reading frame is set forth in FIG. 3 with the corresponding nucleic acid (SEQ ID NO: 9) and amino acid (SEQ ID NO: 10) sequences set forth in FIG. 4A. Note that in FIG. 4A the incorporated sc16.15 scFv binding domain is underlined (corresponding to SEQ ID NO; 8).

### Example 6

### Generation of Additional Exemplary

### Anti-DLL3 Chimeric Antigen Receptors

To further demonstrate the scope and adaptability of the instant invention two DLL3 binding domains, in the form of scFv constructs compatible with the disclosed DLL3 CARs, were fabricated and incorporated into SCT1-16 CARs substantially as set forth above. More specifically, to generate novel anti-DLL3 binding domain constructs in accordance with the instant disclosure nucleotide sequences encoding scFv fragments were synthesized by operably linking selected VL and VH nucleotide sequences together via a nucleic acid sequence encoding a pentameric multimer GlyGlyGlyGlySer (G₄S)₃ (GGGGSGGGGSGGGGS; SEQ ID NO: 7) linker. In the first case the scFv polynucleotide (scFv-hSC16.13) comprises variable light and heavy chain sequences from hSC16.13 (SEQ ID NOS: 388 and 390) while in the second case the scFv polynucleotide (scFv-hSC16.25) comprises variable light and heavy chain sequences from hSC16.25 (SEQ ID NOS: 396 and 398). The resulting nucleic acid constructs encoding scFv-hSC16.13 (SEQ ID NO: 11) and scFv-hSC16.25 (SEQ ID NO: 13) were then inserted in the SCT1 cassette using the engineered restriction sites to provide SCT1-hSC16.13 and SCT1-hSC16.25. The nucleic acid (SEQ ID NO: 16) and amino acid (SEQ ID NO: 17) sequences of SCT1-hSC16.13 are shown in FIG. 4B while the nucleic acid (SEQ ID NO: 18) and amino acid (SEQ ID NO: 19) sequences of SCT1-hSC16.25 are shown in FIG. 4C. Note that in both FIGS. the amino acid sequence corresponding to the DLL3 scFv binding domain is underlined as well as being set forth in SEQ ID NO: 12 (h16.13 scFv) and SEQ ID NO: 14 (h16.25 scFv) respectively.

The ease of fabricating these CARs using the disclosed SCT1 cassette system illustrates the versatility of the instant invention with regard to the selection and incorporation of various DLL3 binding domains and more generally demonstrates the modular nature of the constructs. As such, it will be appreciated that the concept of the DLL3 CARS set forth herein is not limited to any particular DLL3 binding domain or by the selection of any other particular component (e.g. a specific transmembrane or signaling domain) as long as the resulting DLL3 sensitized lymphocyte is immunostimulated or activated by exposure to a DLL3 expressing cell (e.g., a tumor cell).

### Example 7

### Generation and Characterization of Lentiviral Vector Particles

Lentiviral vector packaging of the exemplary DLL3 CARs SCT1-h16.13, SCT1-h16.15 and SCT1-h16.25 from Examples 5 and 6, were carried out as follows: 10 ug of the selected SCT1-h16 plasmid, 7 ug of pAR8.74, and 4 ug of pMD2.G were co-transfected into ten-million HEK-293T cells (ATCC) in the presence of polyethylenemine (Polysciences) at a DNA:PEI ratio of 1:4. Co-transfected cells were incubated at 37° C. (5% CO₂) overnight, followed by media exchange the next day. Forty-eight hours post-transfection, culture media containing lentiviral particles was harvested and clarified by centrifugation at 1200 rpm for 5 min at 4° C. to remove cell debris. To pellet lentiviral vector particles the clarified culture media was ultracentrifuged at 19500 rpm for two hours at 4° C. After ultracentrifugation the supernatant was discarded, the viral pellet resuspended in sterile PBS, and stored at −80° C. Quantitation of recovered lentiviral vector stocks was assessed by p24 ELISA (Cell Biolabs), and gene-transfer efficiency (functional titer) was assessed by standard lentiviral vector titration methods. Typical yields of lentiviral vector stocks ranged from 7-15 ug/ml of p24 antigen, and functional titers ranged from 1-3×10e8 TU/ml. The SCT1-h16.13, SCT1-h16.15 and SCT1-h16.25 lentiviral vector stocks were frozen and stored until use.

As set forth in the subsequent Examples, the vector stocks may be used to induce a desired immune response as discussed in detail throughout the instant application and shown schematically in FIG. 5 appended hereto. Moreover, while SCT1-h16.13, SCT1-h16.15 and SCT1-h16.25 are used as an exemplary constructs to demonstrate various facets of the instant invention (and may be called out in the FIGS.), it will be appreciated that the true scope of the invention is not limited to any particular DLL3 binding domain or particular signaling domain or any exemplary construct thereof, but rather encompasses any DLL3 CAR that effects the desired immune response upon exposure to a DLL3 expressing tumor cell.

### Example 8

### Generation of Immortalized T Lymphocytes

### Expressing SCT1-h16.13, SCT1-h16.15, or SCT1-h16.25 CAR Protein

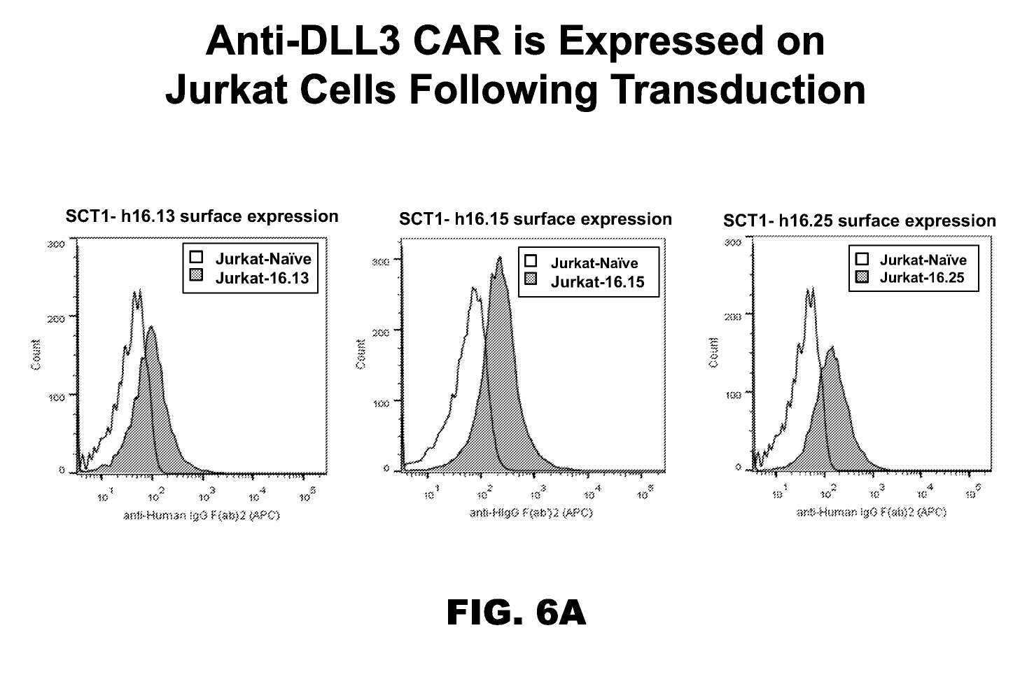

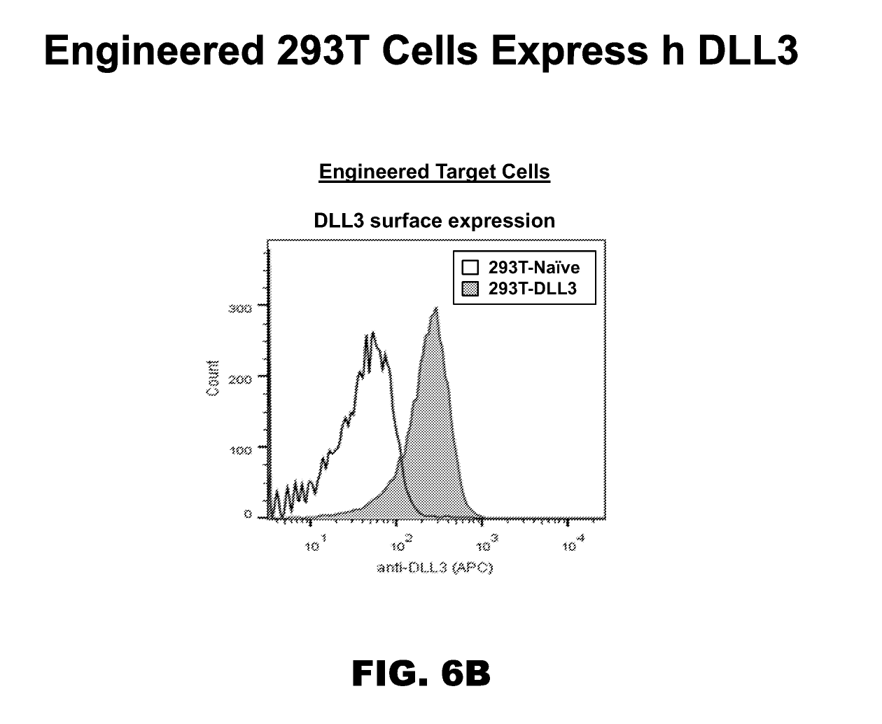

DLL3 target-specific Jurkat lymphocytes expressing either SCT1-h16.13, SCT1-h16.15 or SCT1-h16.25 were generated by transducing one million Jurkat E6-1 (ATCC) T lymphocyte cells with the subject SCT1-h16 lentiviral vector from the previous Example at a multiplicity of infection (MOI) of ˜4 in the presence of 10 ug/ml of polybrene (EMD Millipore) to ensure efficient viral transduction. The cells were allowed to incubate in the presence of lentiviral particles for seventy-two hours at 37° C. (5% CO₂). Afterwards, the spent media was exchanged with fresh media containing 2 ug/ml Puromcyin (Life Technologies) to positively select for SCT1-h16 CAR expressing cells. Cells were allowed to incubate an additional 5 days in the presence of Puromycin prior to assessing the anti-DLL3 CAR surface expression by flow cytometry (FIG. 6A). Flow cytometry was also used to detect the presence of hDLL3 protein on the surface of an engineered HEK-293T cell line overexpressing hDLL3 (FIG. 6B) that will be used to characterize CAR constructs of the instant invention.

Flow cytometry analysis of transduced Jurkat cells expressing either SCT1-h16.13, SCT1-h16.15 or SCT1-h16.25 and non-transduced Jurkat control cells was performed as follows: 10⁶ cells of each cell line were harvested and pelleted by centrifugation at 1200 rpm at 4° C. for 5 minutes; supernatant was removed and the pellet was washed in cold PBS/2% FCS twice. After supernatant from the final wash was removed, the cell pellet was resuspended in 100 microliters of PBS/2% FCS containing 1 microgram of Alexa Fluor® 647-conjugated Affinipure Goat Anti-Human IgG, F(ab′) antibody (Jackson ImmunoResearch) and incubated in the dark at 4° C. for 30 minutes. After incubation, cells were washed three times in PBS/2% FCS before being re-suspended in PBS/2% FCS with DAPI (to detect living cells). The cells were then analyzed on a BD FACS Canto II flow cytometer as per the manufacturer's instructions to provide the data set forth in FIG. 6A.

Similarly, HEK-293T parental cells or HEK-293T cells overexpressing hDLL3 were harvested and isolated into single cell suspensions with Versene (Life Technologies). The isolated cells were washed as described above and incubated for 30 minutes at 4° C. in the dark with 1 microgram of anti-DLL3 antibody prior to thrice washing in PBS/2% FCS. The cells were then incubated for 30 minutes with 50 μL per sample AlexaFluor-647 labeled goat-anti-mouse IgG, Fc fragment specific secondary antibody (Life Technologies) diluted 1:200 in PBS/2% FCS, washed thrice with PBS/2% FCS and re-suspended in PBS/2% FCS with DAPI (to detect living cells). The cells were then analyzed on a BD FACS Canto II flow cytometer as per the manufacturer's instructions to provide the data set forth in FIG. 6B.

FIGS. 6A and 6B, respectively, demonstrate that the subject SCT1-h16 CAR is expressed on transduced Jurkat T lymphocytes but not on non-transduced Jurkat cells, and that human DLL3 protein is expressed on the engineered HEK-293T cells but not on HEK-293T-Naïve cells.

### Example 9

### Jurkat-SCT1-h16.15 T Lymphocytes Induce

### IL-2 Production Upon Contacting hDLL3 Expressing Cells

Transduced Jurkat-SCT1-h16.13, Jurkat-SCT1-h16.15, and Jurkat-SCT1-h16.25 lymphocytes were assessed for target-specific activation by measuring IL-2 induction which is indicative of CAR mediated T-cell activation. More specifically, using transduced Jurkat lymphocytes and engineered 293T cells expressing hDLL3 from the previous Example, IL2 levels were monitored to demonstrate that the CAR expressing lymphocytes are activated and mount an immune response upon contact with cells expressing hDLL3.

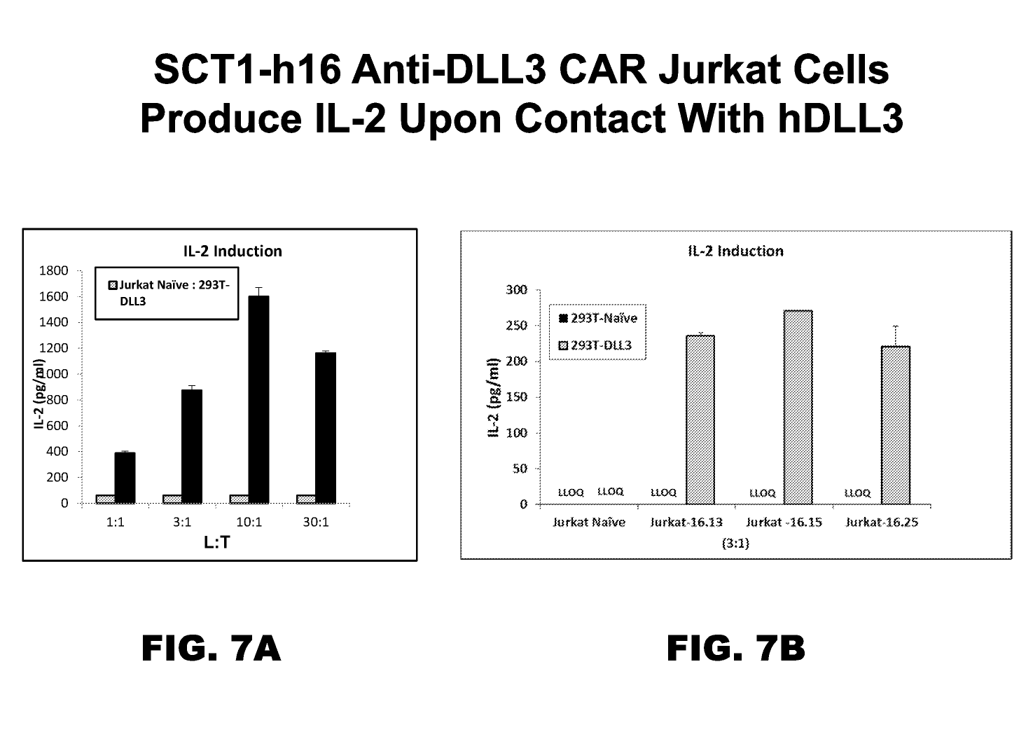

In this regard Jurkat-SCT1-h16.15 lymphocytes from Example 8 were co-cultured with HEK-293T cells engineered to over-express hDLL3 antigen on the cell surface (also from Example 8) as evidenced by flow cytometry. Co-culturing of lymphocytes with target HEK-293T-hDLL3 cells was performed at the four different lymphocyte: target (L:T) ratios set forth in FIG. 7A to assess dose response and determine maximum IL-2 production conditions. Co-cultures were incubated at 37° C. (5% CO₂) for 48 hrs, at which time media was harvested and clarified of cell debris by centrifugation at 1200 rpm for 5 minutes. Clarified supernatant was then assessed for IL-2 production by ELISA (Thermo Scientific) per manufacturer's instructions. To assess background IL-2 production, non-transduced Jurkat cells (Jurkat-Naïve) were co-cultured with HEK-293T-hDLL3 cells. The results, in terms of IL2 induction, is shown in FIG. 7A.

Similarly three different SCT1-h16 lymphocytes (Jurkat-SCT1-h16.13, Jurkat-SCT1-h16.15, and Jurkat-SCT1-h16.25) from Example 8 were co-cultured with engineered hDLL3+HEK-293T cells at a 3:1 L:T ratio substantially as set forth immediately above. Results, again in terms of IL2 induction, are shown in FIG. 7B.

As evidenced by the data set forth in FIG. 7A, the Jurkat-SCT1-h16.15 lymphocytes were prompted to produce IL-2 in a concentration dependent manner upon exposure to cells expressing hDLL3. Additionally, data shown in FIG. 7B demonstrates the ability of various DLL3 CAR constructs to consistently induce the production of IL2 when exposed to antigen presenting cells. More particularly it will be appreciated that such IL-2 production is indicative of T-cell activation by the SCT1 CAR upon recognition of DLL3 antigen on hDLL3 expressing cells (including hDLL3 expressing tumorigenic cells). With regard to both FIGS. 7A and 7B target-specific CAR-mediated activation of Jurkat cells is further elucidated by the lack of observable IL-2 production among co-cultures containing HEK-293T-DLL3 and non-transduced Jurkat cells.

### Example 10

### Generation of Primary T Lymphocytes Expressing

### SCT1-h16.13, SCT1-h16.15, or SCT1-h16.25 CAR Protein

In order to demonstrate that the disclosed CARs may be used to provide sensitized lymphocytes primary human CD3+T lymphocytes were isolated from a commercially available peripheral blood mononuclear cell preparation (PBMCs: AIICells) using a human CD3 positive selection kit (Stemcell Technologies).

Following isolation from a single donor CD3+ T cells were cultured in RPMI media containing 10% heat-inactivated fetal bovine serum (Hyclone), 1% penicillin/streptomycin (Corning), 1% I-glutamine (Corning), and 10 mM HEPES (Corning). T lymphocytes were incubated at 37° C. (5% CO₂) in the presence of CD3/CD28 activation beads (Dynabeads) at a 1:5 ratio for activation. IL-2 (Peprotech) was added every other day to a final concentration of 50 IU/ml. Twenty-four hours after initial activation, DLL3 target-specific T lymphocytes expressing SCT1-h16.13, SCT1-h16.15, or SCT1-h16.25 were generated by transducing one million T cells with the CAR lentiviral vectors generated substantially as set forth in Example 7 at a multiplicity of infection (MOI) of ˜5 in the presence of 10 ug/ml of polybrene (EMD Millipore) to ensure efficient viral transduction. The cells were allowed to incubate in the presence of lentiviral particles for seventy-two hours at 37° C. (5% CO₂) prior to assessing the anti-DLL3 CAR surface expression by flow cytometry.

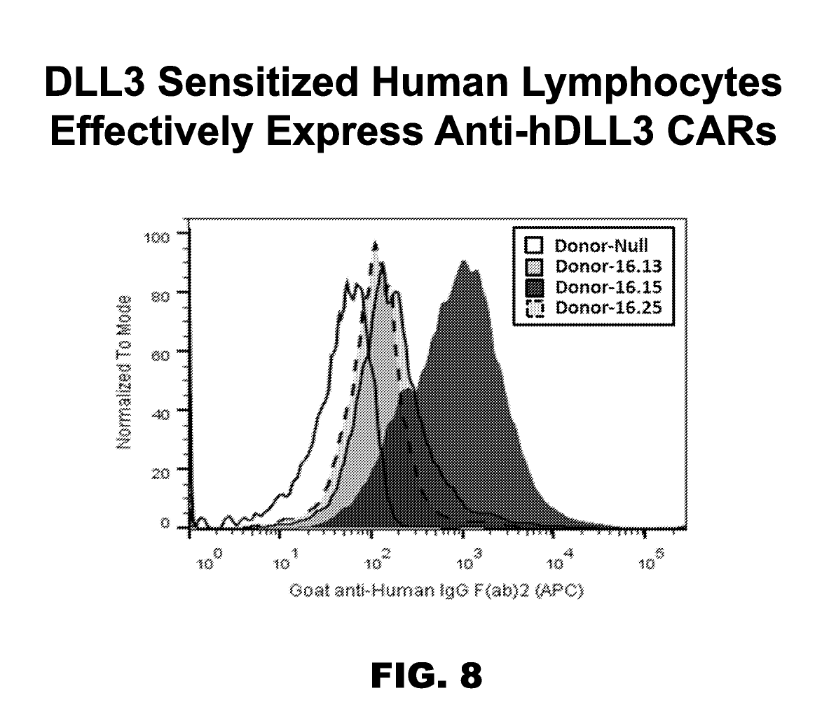

Flow cytometry analysis of transduced T lymphocytes expressing SCT1-h16.13, SCT1-h16.15 or SCT1-h16.25 and non-CAR-bearing T lymphocyte control cells was performed as follows: 10⁶ cells of each sample were harvested and pelleted by centrifugation at 1200 rpm at 4° C. for 5 minutes; supernatant was removed and the pellet was washed in cold PBS/2% FCS twice. After supernatant from the final wash was removed, the cell pellet was resuspended in 100 microliters of PBS/2% FCS containing 1 microgram of Alexa Fluor® 647-conjugated Affinipure Goat Anti-Human IgG, F(ab′) antibody (Jackson ImmunoResearch) and incubated in the dark at 4° C. for 30 minutes. After incubation, cells were washed thrice in PBS/2% FCS before being re-suspended in PBS/2% FCS with DAPI (to detect living cells). The cells were then analyzed on a BD FACS Canto II flow cytometer as per the manufacturer's instructions to provide the data set forth in FIG. 8.

FIG. 8 clearly shows that SCT1-h16.13, SCT1-h16.15 and SCT1-h16.25 are expressed on transduced primary T lymphocytes (i.e., sensitized lymphocytes) but not on non-transduced lymphocytes.

### Example 11

### Generation and Characterization of Cells Expressing DLL3 Target Antigen

As set forth in Example 8 flow cytometry was used to detect the presence of DLL3 protein on the surface of an engineered HEK-293T cell line overexpressing human DLL3. Similarly flow cytometry was used to confirm the expression of human DLL3 on a patient-derived xenograph (PDX) tumor cell line (LU64). Both the artificially engineered 293T cell line and the derived small cell cancer cell line were used to characterize sensitized lymphocytes of the instant invention.

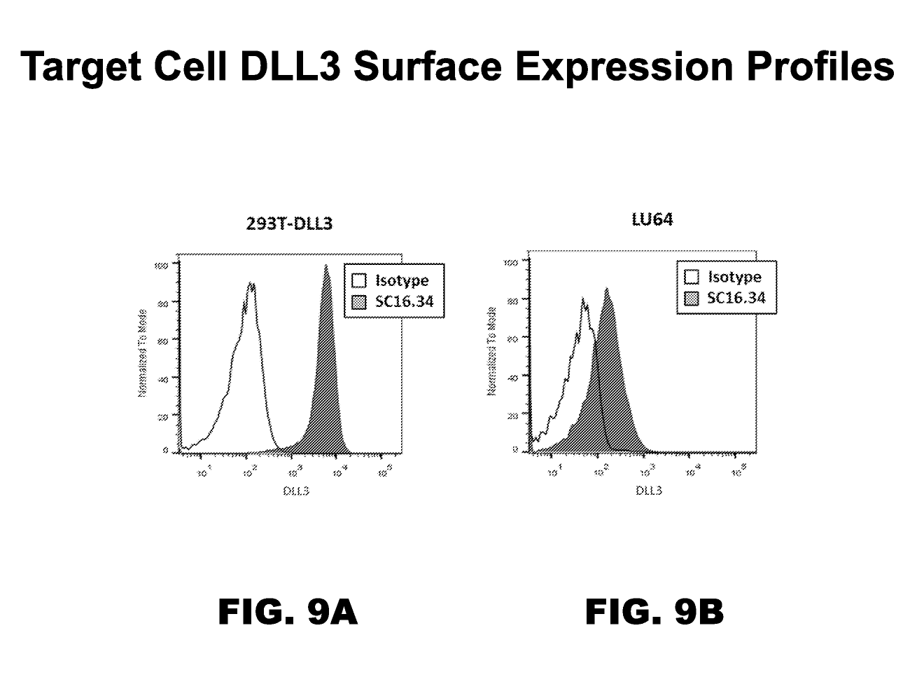

More particularly HEK-293T cells overexpressing human DLL3 (293T-DLL3) were harvested and isolated into single cell suspensions with Versene (Life Technologies). Similarly, freshly harvested LU64 PDX tumors were processed into single cell suspension using a tumor dissociation kit (Mylteni Biotec). Isolated cells were washed as described herein and incubated for 30 minutes at 4° C. in the dark with 1 microgram of anti-DLL3 antibody or isotype control prior to thrice washing in PBS/2% FCS. The cells were then incubated for 30 minutes with 50 microliters per sample of AlexaFluor-647 labeled goat-anti-mouse IgG, Fc fragment specific secondary antibody (Life Technologies) diluted 1:200 in PBS/2% FCS, washed thrice with PBS/2% FCS and resuspended in PBS/2% FCS with DAPI (to detect living cells). Cells were then analyzed on a BD FACS Canto II flow cytometer as per the manufacturer's instructions to provide the data set forth in FIGS. 9A and 9B.

The resulting FIGS. show that human DLL3 protein is expressed both on the engineered HEK-293T cells (FIG. 9A) and LU64 PDX tumor cells (FIG. 9B).

### Example 12

### SCT1-h16.13, SCT1-h16.15 and SCT1-h16.25

### Primary Lymphocytes Eliminate DLL3 Expressing Cells Upon Exposure

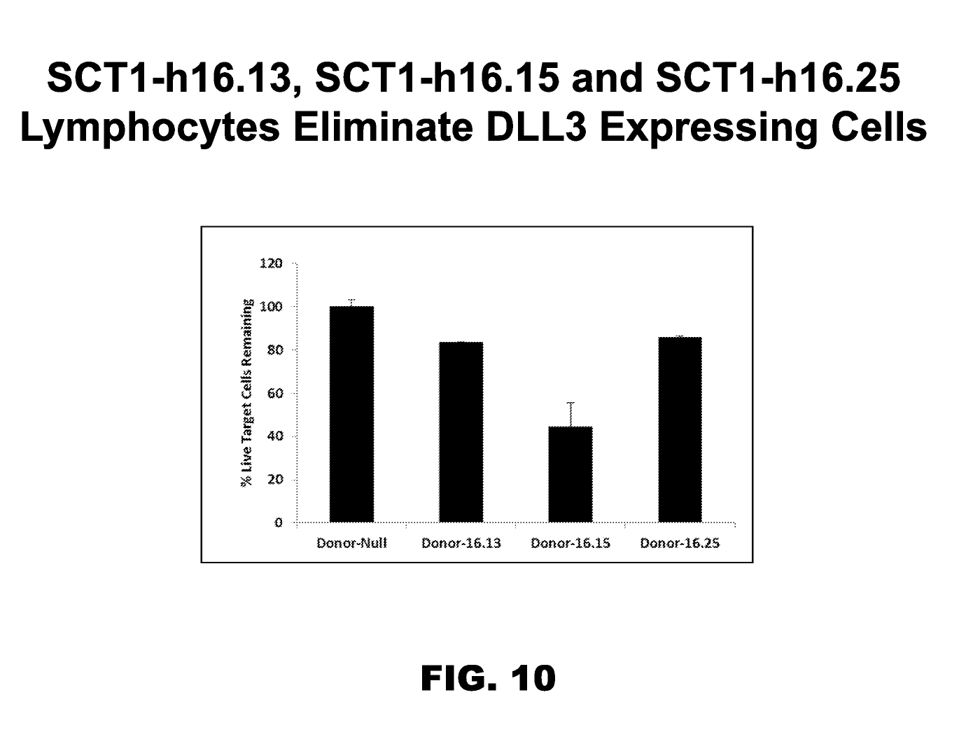

To demonstrate the ability of DLL3 sensitized primary lymphocytes to kill cells in a target-specific manner, CAR transduced cells of the instant invention (prepared substantially as set forth in Example 10) were exposed to engineered 293 cells expressing DLL3 (along with appropriate controls). Following exposure the number of living target cells remaining were calculated with the results being set forth in FIG. 10.

More specifically primary T lymphocytes expressing anti-DLL3 CARs (SCT1-h16.13, SCT1-h16.15 and SCT1-h16.25) were co-cultured with 293T-DLL3 cells at an lymphocyte-to-target (L:T) ratio of 3:1. Co-cultures were incubated at 37° C. (5% CO₂) for 48 hrs prior to determination percentage of remaining viable DLL3-bearing cells.

The percentage of live cells was calculated as follows: co-cultures were harvested and washed as set forth herein prior to incubation for 30 minutes at 4° C. in the dark with 1 microgram of anti-DLL3 antibody or isotype control followed by thrice washing in PBS/2% FCS. Cells were then incubated for 30 minutes with 50 microliters per sample of AlexaFluor-647 labeled goat-anti-mouse IgG, Fc fragment-specific secondary antibody (Life Technologies) diluted 1:200 in PBS/2% FCS. Cells were washed three times with PBS/2% FCS, followed by resuspension in 200 microliters of PBS/2% FCS containing DAPI (Life Technologies) for cell viability discrimination, and 10000 absolute counting beads (Life Technologies) for normalizing cell counts. Analysis and enumeration of remaining viable DLL3-bearing target cells was performed on a BD FACS Canto II flow cytometer by quantifying the respective number of viable DLL3-bearing cells per 7500 absolute counting beads collected. Viable target cells remaining in presence of non-CAR-bearing T lymphocytes was used as the benchmark to compare target-specific killing of DLL3-bearing cells by DLL3 sensitized lymphocytes.

As shown in FIG. 10, SCT1-16.15 exhibited significant target-specific killing while SCT1-h16.13 and SCT1-h16.25 showed more moderate target-specific killing. The immunospecificity of killing mediated by the sensitized lymphocytes is evidenced by the lack of activity shown by the non-transduced primary T lymphocytes co-cultured with DLL3+ target cells (FIG. 10). These data demonstrate the target-specific activity among sensitized lymphocytes expressing various anti-DLL3 CARs.

### Example 13

### SCT1-h16.15 Primary T Lymphocytes

### Induce Cytokine Production Upon Contacting DLL3-Expressing Cells

In order to demonstrate that the disclosed CARs may be used to provide sensitized lymphocytes from various donors, primary human CD3+T lymphocytes were isolated from commercially available peripheral blood mononuclear cell preparations (PBMCs: AIICells) using a human CD3 positive selection kit (Stemcell Technologies) and transduced. The resulting sensitized lymphocyte compositions comprising SCT1-h16.15 and PBMCs obtained from two different donors (donor 1 and donor 2) were assessed for CAR expression (FIG. 11) and target-specific activation by measuring TNFα and IFNγ induction upon contact with DLL3-expressing target cells. It will be appreciated that cytokine production (e.g., TNFα and IFNγ induction) is indicative of active chimeric antigen receptors that are capable of inducing an anti-tumor immune response.

More particularly, PMBC preparations from two different donors (donor 1 and donor 2) were used to provide CD3+T lymphocyte preparations substantially as set forth in Example 10. The respective lymphocyte preparations were then transduced with SCT1-h16.15 (again as set forth in Example 10) to provide donor 1 and donor 2 DLL3 sensitized lymphocyte preparations (along with non-transduced lymphocytes as controls). FIG. 11 shows that each of the lymphocyte preparations effectively express the DLL3 CAR as determined by flow cytometry performed as set forth above.

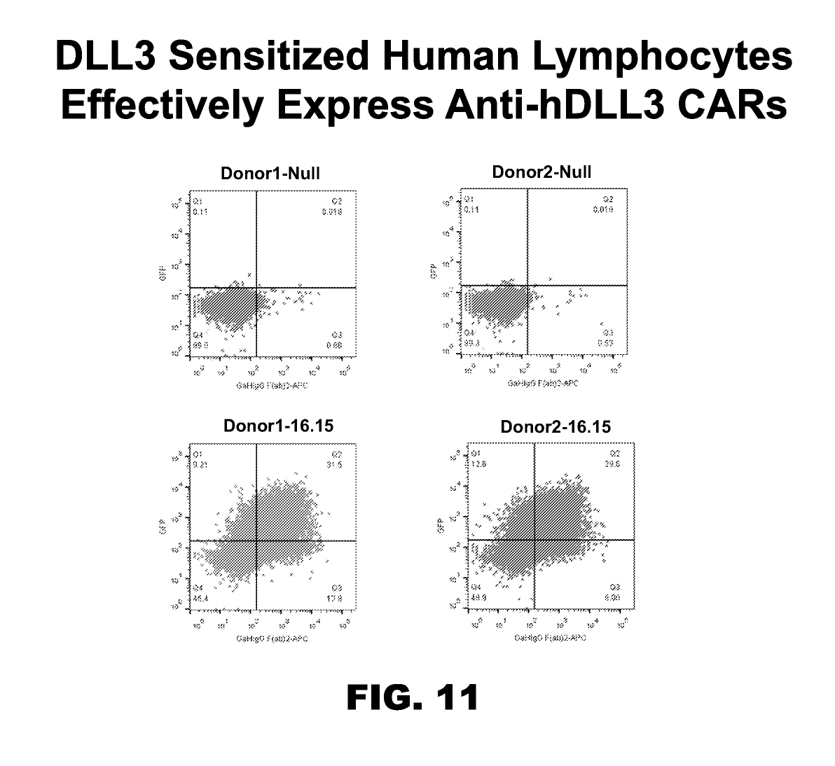

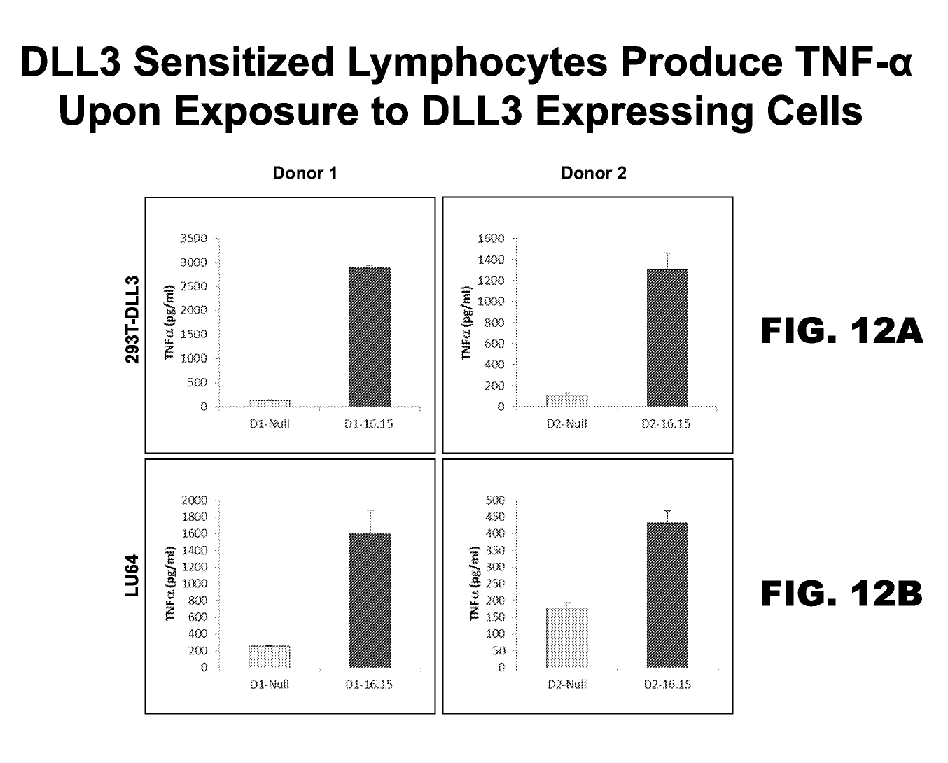

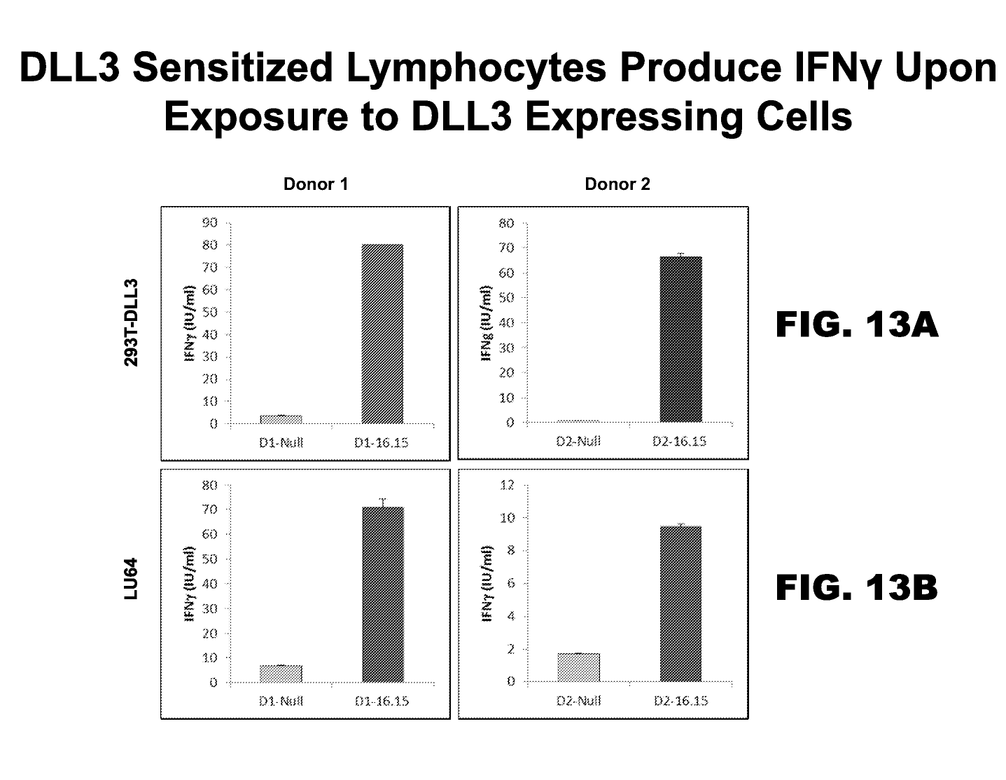

The resulting sensitized lymphocytes comprising host cells from the two different donors were the exposed to DLL3+293T cells and small cell lung cancer cells expressing DLL3 (both from Example 11). In this regard each of the sensitized lymphocyte preparations (with controls) were then co-cultured with either 293T-DLL3 or LU64 PDX target cells at a lymphocyte to target (L:T) ratio of 3:1. Co-cultures were incubated at 37° C. (5% CO₂) for 48 hrs, at which time media was harvested and clarified of cell debris by centrifugation at 1200 rpm for 5 minutes. Clarified supernatant was then assessed for TNFα production by ELISA (Thermo Fisher) and IFNg by ELISA (Invitrogen) per manufacturer's instructions. The resulting measurements for levels of TNFα and IFNγ are shown, respectively, in FIGS. 12A and 12B (TNFα) and FIGS. 13A and 13B (IFNγ) where higher cytokine production is indicative of more robust signaling from the CAR.

As evidenced by the data set forth in FIG. 12A (293 cells) and 12B (tumor cells) and FIG. 13A (293 cells) and 13B (tumor cells) both preparations of the SCT1-h16.15-bearing T lymphocytes were prompted to produce TNFα and IFNγ upon exposure to cells (engineered and tumor) expressing human DLL3, whereas the non-CAR-bearing T lymphocytes exhibited minimal TNFα and IFNγ induction when co-cultured with the same target cells. This confirms that DLL3 sensitized lymphocytes from different donors are active and capable of generating immunostimulatory signals upon exposure to DLL3+ tumor cells.

### Example 14

### Targeted Killing of DLL3-Expressing Cells In Vitro by SCT1-h16.15-T Lymphocytes

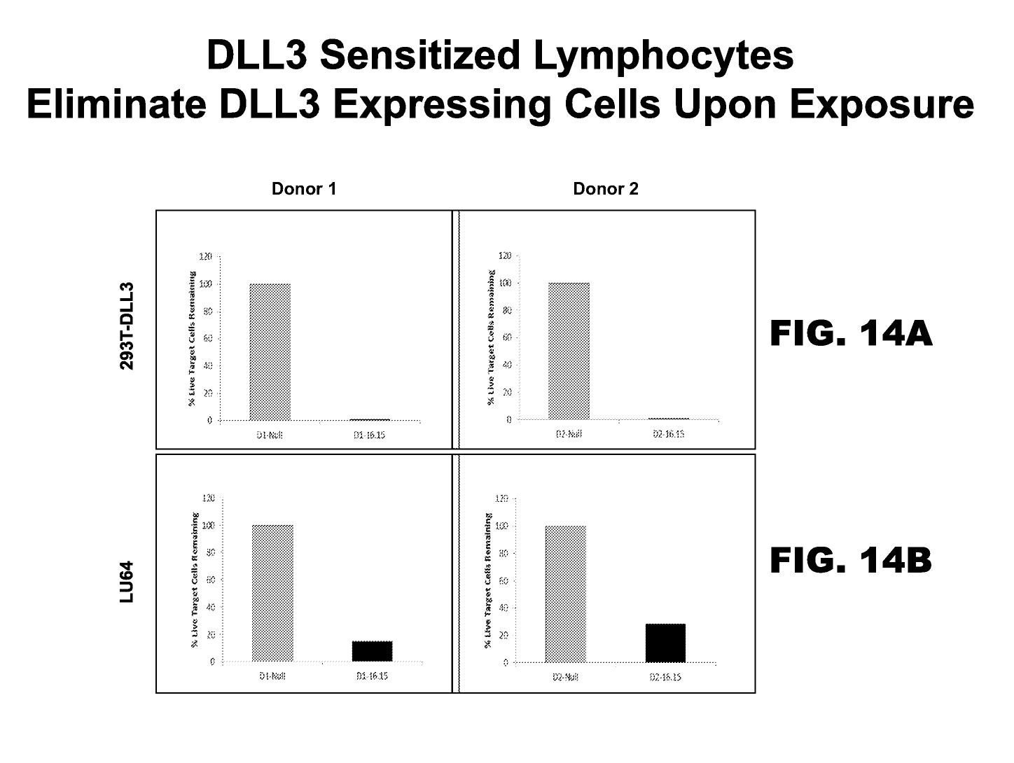

To demonstrate the ability of DLL3 sensitized lymphocytes to kill cells in a target-specific manner, CAR transduced cells of the instant invention were exposed to engineered 293 cells and tumor cells expressing DLL3 (again from Example 11). Following exposure the number of living target cells remaining were calculated with the results being set forth in FIG. 14A (293 cells) and 14B (tumor cells).

More particularly SCT1-h16.15 sensitized lymphocytes (prepared as per Example 13 with host cells from two donors) were co-cultured with either 293T-DLL3 or LU64 PDX cells at an lymphocyte to target (L:T) ratio of 3:1. Co-cultures were incubated at 37° C. (5% CO₂) for 48 hrs prior to determination of remaining viable DLL3-bearing cells.

The percentage of live cells was calculated as follows: co-cultures were harvested and washed as set forth herein prior to incubation for 30 minutes at 4° C. in the dark with 1 microgram of anti-DLL3 antibody or isotype control followed by thrice washing in PBS/2% FCS. Cells were then incubated for 30 minutes with 50 microliters per sample of AlexaFluor-647 labeled goat-anti-mouse IgG, Fc fragment-specific secondary antibody (Life Technologies) diluted 1:200 in PBS/2% FCS. Cells were washed three times with PBS/2% FCS, followed by resuspension in 200 microliters of PBS/2% FCS containing DAPI (Life Technologies) for cell viability discrimination, and 10000 absolute counting beads (Life Technologies) for normalizing cell counts. Analysis and enumeration of remaining viable DLL3-bearing target cells was performed on a BD FACS Canto II flow cytometer by quantifying the respective number of viable DLL3-bearing cells per 7500 absolute counting beads collected. Viable target cells remaining in presence of non-CAR-bearing T lymphocytes was used as the benchmark to compare target-specific killing of DLL3-bearing cells by SCT1-h16.15 sensitized lymphocytes.

As shown in FIG. 14A (293 cells) and 14B (tumor cells) the DLL3+ cells exhibited significant susceptibility to cytolysis by the sensitized lymphocytes derived from both donors with approximately 99% of target cells being eliminated. The DLL3 sensitized lymphocytes were also able to eliminate a substantial majority (approximately 70% to 80%) of LU64 PDX small cell lung cancer cells. Overall, these data demonstrate that the disclosed DLL3 sensitized lymphocytes are able to effectively eliminate DLL3+ cells, including DLL3+ tumor cells in a target-specific manner.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1. A chimeric antigen receptor comprising an anti-DLL3 binding domain.

2. The chimeric antigen receptor of claim 1 wherein the anti-DLL3 binding domain comprises a scFv anti-DLL3 binding domain.

3. The chimeric antigen receptor of claim 1 or 2 wherein the scFv anti-DLL3 binding domain is derived from, comprises or competes for binding with an antibody comprising: a light chain variable region (VL) of SEQ ID NO: 21 and a heavy chain variable region (VH) of SEQ ID NO: 23; or a VL of SEQ ID NO: 25 and a VH of SEQ ID NO: 27; or a VL of SEQ ID NO: 29 and a VH of SEQ ID NO: 31; or a VL of SEQ ID NO: 33 and a VH of SEQ ID NO: 35; or a VL of SEQ ID NO: 37 and a VH of SEQ ID NO: 39; or a VL of SEQ ID NO: 41 and a VH of SEQ ID NO: 43; or a VL of SEQ ID NO: 45 and a VH of SEQ ID NO: 47; or a VL of SEQ ID NO: 49 and a VH of SEQ ID NO: 51; or a VL of SEQ ID NO: 53 and a VH of SEQ ID NO: 55; or a VL of SEQ ID NO: 57 and a VH of SEQ ID NO: 59; or a VL of SEQ ID NO: 61 and a VH of SEQ ID NO: 63; or a VL of SEQ ID NO: 65 and a VH of SEQ ID NO: 67; or a VL of SEQ ID NO: 69 and a VH of SEQ ID NO: 71; or a VL of SEQ ID NO: 73 and a VH of SEQ ID NO: 75; or a VL of SEQ ID NO: 77 and a VH of SEQ ID NO: 79; or a VL of SEQ ID NO: 81 and a VH of SEQ ID NO: 83; or a VL of SEQ ID NO: 85 and a VH of SEQ ID NO: 87; or a VL of SEQ ID NO: 89 and a VH of SEQ ID NO: 91; or a VL of SEQ ID NO: 93 and a VH of SEQ ID NO: 95; or a VL of SEQ ID NO: 97 and a VH of SEQ ID NO: 99; or a VL of SEQ ID NO: 101 and a VH of SEQ ID NO: 103; or a VL of SEQ ID NO: 105 and a VH of SEQ ID NO: 107; or a VL of SEQ ID NO: 109 and a VH of SEQ ID NO: 111; or a VL of SEQ ID NO: 113 and a VH of SEQ ID NO: 115; or a VL of SEQ ID NO: 117 and a VH of SEQ ID NO: 119; or a VL of SEQ ID NO: 121 and a VH of SEQ ID NO: 123; or a VL of SEQ ID NO: 125 and a VH of SEQ ID NO: 127; or a VL of SEQ ID NO: 129 and a VH of SEQ ID NO: 131; or a VL of SEQ ID NO: 133 and a VH of SEQ ID NO: 135; or a VL of SEQ ID NO: 137 and a VH of SEQ ID NO: 139; or a VL of SEQ ID NO: 141 and a VH of SEQ ID NO: 143; or a VL of SEQ ID NO: 145 and a VH of SEQ ID NO: 147; or a VL of SEQ ID NO: 149 and a VH of SEQ ID NO: 151; or a VL of SEQ ID NO: 153 and a VH of SEQ ID NO: 155; or a VL of SEQ ID NO: 157 and a VH of SEQ ID NO: 159; or a VL of SEQ ID NO: 161 and a VH of SEQ ID NO: 163; or a VL of SEQ ID NO: 165 and a VH of SEQ ID NO: 167; or a VL of SEQ ID NO: 169 and a VH of SEQ ID NO: 171; or a VL of SEQ ID NO: 173 and a VH of SEQ ID NO: 175; or a VL of SEQ ID NO: 177 and a VH of SEQ ID NO: 179; or a VL of SEQ ID NO: 181 and a VH of SEQ ID NO: 183; or a VL of SEQ ID NO: 185 and a VH of SEQ ID NO: 187; or a VL of SEQ ID NO: 189 and a VH of SEQ ID NO: 191; or a VL of SEQ ID NO: 193 and a VH of SEQ ID NO: 195; or a VL of SEQ ID NO: 197 and a VH of SEQ ID NO: 199; or a VL of SEQ ID NO: 201 and a VH of SEQ ID NO: 203; or a VL of SEQ ID NO: 205 and a VH of SEQ ID NO: 207; or a VL of SEQ ID NO: 209 and a VH of SEQ ID NO: 211; or a VL of SEQ ID NO: 213 and a VH of SEQ ID NO: 215; or a VL of SEQ ID NO: 217 and a VH of SEQ ID NO: 219; or a VL of SEQ ID NO: 221 and a VH of SEQ ID NO: 223; or a VL of SEQ ID NO: 225 and a VH of SEQ ID NO: 227; or a VL of SEQ ID NO: 229 and a VH of SEQ ID NO: 231; or a VL of SEQ ID NO: 233 and a VH of SEQ ID NO: 235; or a VL of SEQ ID NO: 237 and a VH of SEQ ID NO: 239; or a VL of SEQ ID NO: 241 and a VH of SEQ ID NO: 243; or a VL of SEQ ID NO: 245 and a VH of SEQ ID NO: 247; or a VL of SEQ ID NO: 249 and a VH of SEQ ID NO: 251; or a VL of SEQ ID NO: 253 and a VH of SEQ ID NO: 255; or a VL of SEQ ID NO: 257 and a VH of SEQ ID NO: 259; or a VL of SEQ ID NO: 261 and a VH of SEQ ID NO: 263; or a VL of SEQ ID NO: 265 and a VH of SEQ ID NO: 267; or a VL of SEQ ID NO: 269 and a VH of SEQ ID NO: 271; or a VL of SEQ ID NO: 273 and a VH of SEQ ID NO: 275; or a VL of SEQ ID NO: 277 and a VH of SEQ ID NO: 279; or a VL of SEQ ID NO: 281 and a VH of SEQ ID NO: 283; or a VL of SEQ ID NO: 285 and a VH of SEQ ID NO: 287; or a VL of SEQ ID NO: 289 and a VH of SEQ ID NO: 291; or a VL of SEQ ID NO: 293 and a VH of SEQ ID NO: 295; or a VL of SEQ ID NO: 297 and a VH of SEQ ID NO: 299; or a VL of SEQ ID NO: 301 and a VH of SEQ ID NO: 303; or a VL of SEQ ID NO: 305 and a VH of SEQ ID NO: 307; or a VL of SEQ ID NO: 309 and a VH of SEQ ID NO: 311; or a VL of SEQ ID NO: 313 and a VH of SEQ ID NO: 315; or a VL of SEQ ID NO: 317 and a VH of SEQ ID NO: 319; or a VL of SEQ ID NO: 321 and a VH of SEQ ID NO: 323; or a VL of SEQ ID NO: 325 and a VH of SEQ ID NO: 327; or a VL of SEQ ID NO: 329 and a VH of SEQ ID NO: 331; or a VL of SEQ ID NO: 333 and a VH of SEQ ID NO: 335; or a VL of SEQ ID NO: 337 and a VH of SEQ ID NO: 339; or a VL of SEQ ID NO: 341 and a VH of SEQ ID NO: 343; or a VL of SEQ ID NO: 345 and a VH of SEQ ID NO: 347; or a VL of SEQ ID NO: 349 and a VH of SEQ ID NO: 351; or a VL of SEQ ID NO: 353 and a VH of SEQ ID NO: 355; or a VL of SEQ ID NO: 357 and a VH of SEQ ID NO: 359; or a VL of SEQ ID NO: 361 and a VH of SEQ ID NO: 363; or a VL of SEQ ID NO: 365 and a VH of SEQ ID NO: 367; or a VL of SEQ ID NO: 369 and a VH of SEQ ID NO: 371; or a VL of SEQ ID NO: 373 and a VH of SEQ ID NO: 375; or a VL of SEQ ID NO: 377 and a VH of SEQ ID NO: 379; or a VL of SEQ ID NO: 381 and a VH of SEQ ID NO: 383; or a VL of SEQ ID NO: 385 and a VH of SEQ ID NO: 387; or a VL of SEQ ID NO: 389 and a VH of SEQ ID NO: 391; or a VL of SEQ ID NO: 393 and a VH of SEQ ID NO: 395; or a VL of SEQ ID NO: 397 and a VH of SEQ ID NO: 399; or a VL of SEQ ID NO: 401 and a VH of SEQ ID NO: 403; or a VL of SEQ ID NO: 405 and a VH of SEQ ID NO: 407.

4. The chimeric antigen receptor of any one of claims 1-3 comprising an intracellular domain comprising a 4-1BB signaling domain and/or a CD3ζ signaling domain.

5. The chimeric antigen receptor of any one of claims 1-4 comprising a transmembrane domain comprising a human CD8a hinge.

6. A pharmaceutical composition comprising a chimeric antigen receptor of any one of claims 1-5 and a pharmaceutically acceptable carrier

7. A polynucleotide encoding a chimeric antigen receptor of any one of claims 1 to 5.

8. A vector comprising a polynucleotide of claim 7.

9. The vector of claim 8 wherein the vector comprises a viral vector.

10. The vector of claim 9 wherein the viral vector comprises a lentiviral vector or a retroviral vector.

11. A pharmaceutical composition comprising a polynucleotide of claim 7 or a vector of any one of claims 8 to 10.

12. An isolated host cell comprising a chimeric antigen receptor of any one of claims 1 to 5.

13. The isolated host cell of claim 12 wherein the host cell comprises a DLL3 sensitized lymphocyte.

14. The isolated host cell of claim 13 wherein the DLL3 sensitized lymphocyte comprises an autologous cell obtained from a patient.

15. The isolated host cell of claim 13 wherein the DLL3 sensitized lymphocyte comprises an allogeneic cell.

16. The isolated host cell of claim 13 wherein the DLL3 sensitized lymphocyte comprises a T cell or a NK cell.

17. The isolated host cell of claim 16 wherein the T cell comprises a CD8+ T cell.

18. The isolated host cell of claim 16 wherein the DLL3 sensitized lymphocyte comprises a NK cell.

19. A pharmaceutical composition comprising a host cell of any one of claims 12 to 18.

20. A method of treating a patient suffering from cancer comprising the step of administering a pharmaceutical composition of claim 19.

21. The method of claim 20 wherein the patient is suffering from a cancer selected from the group consisting of lung cancer, melanoma, breast cancer, prostate cancer, colon cancer, renal cell carcinoma, ovarian cancer, neuroblastoma, rhabdomyosarcoma, leukemia and lymphoma.

22. The method of claim 21 wherein the cancer is lung cancer and the lung cancer is small cell lung cancer.

23. A method of producing a DLL3 sensitized lymphocyte comprising the step of transforming a lymphocyte with a DLL3 CAR.

24. A method of producing a DLL3 sensitized lymphocyte comprising the step of transducing a lymphocyte with a DLL3 CAR.

25. A method of producing a DLL3 sensitized lymphocyte comprising the step of transfecting a lymphocyte with a DLL3 CAR.

26. An article of manufacture comprising a receptacle containing a DLL3 sensitized lymphocyte and a pharmaceutically acceptable carrier.

27. An article of manufacture comprising a receptacle containing a pharmaceutical composition of claim 11.

28. The article of manufacture of claim 27 wherein the pharmaceutical composition comprises a viral vector.

29. An article of manufacture comprising a receptacle containing a pharmaceutical composition of claim 19.

30. The article of manufacture of any one of claims 26 to 29 wherein the receptacle comprises a syringe, an infusion bag or a vial.
